(19) United States
(12) Patent Application Publication
(10) Pub. No.: US 2008/0026394 A1
(43) Pub. Date: Jan. 31, 2008

(54) Methods of detecting one or more cancer markers

(75) Inventors: Marc R. Labgold, Reston, VA (US); George G. Jokhadze, Mountain View, CA (US)

Correspondence Address: PATTON BOGGS LLP, 8484 WESTPARK DRIVE, SUITE 900, MCLEAN, VA, 22102, US

(73) Assignee: Antara Biosciences Inc., Mountain View, CA (US)

(21) Appl. No.: 11/822,886

(22) Filed: Jul. 11, 2007

Related U.S. Application Data
(60) Provisional application No. 60/830,129, filed on Jul. 11, 2006.

(51) Int. Cl. C12Q 1/68

(52) U.S. Cl. 435/6

(57) ABSTRACT

The present invention provides a methods and compositions for early diagnosis of cancer by rapid and specific detection of one or more cancer markers in a sample.

### CROSS REFERENCE TO RELATED APPLICATIONS

This application claims the benefit of U.S. Provisional Patent Application Ser. No. 60/830,129, filed Jul. 11, 2006, currently pending, which is herein incorporated by reference in its entirety for all purposes.

### FIELD OF THE INVENTION

This invention relates to methods and compositions capable of rapid diagnosis of cancers as well as kits for performing such diagnosis.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Enzyme-linked immunosorbent assay (ELISA) is a widely used method for measuring the concentration of a particular molecule (e.g., a hormone or drug) in a fluid such as serum or urine. It is also known as enzyme immunoassay or EIA. The molecule or target agent is detected by antibodies that have been made against it; that is, for which it is the antigen. Monoclonal antibodies are often used. Due to the diversity found in the immune system and the production of monoclonal antibodies from immortalized cells of the immune system, first described by Kohler and Milstein in 1975, antibodies can be raised against a huge number of different antigens by standard immunological techniques. Potentially any target agent can be recognized by a specific antibody that will not react with any other target agent.

An ELISA typically involves coating a vessel, such as a microtiter plate with an antibody specific for a particular antigen to be detected, e.g., a molecule derived from a virus or bacteria, adding the sample suspected of containing the particular antigen, allowing the antibody to bind the antigen and then adding at least one other antibody specific to another region of the same antigen to be detected. This use of two antibodies can be referred to as a “sandwich” ELISA. Sometimes, the second antibody or even a third antibody is used that is labeled with a chromogenic or fluorogenic reporter molecule to aid in detection. The procedure may also involve the need for a chemical substrate to produce a signal. The need for multiple antibodies, which do not cross-react with other antigens, and the incubation steps involved mean that it is difficult to detect more than a single antigen in a sample in a short time period.

Another method of detecting the presence of particular target agents in a sample involves detecting the presence of nucleic acids. Several methods of detecting nucleic acids are available including PCR and hybridization techniques. PCR is well known in the art and is described in U.S. Pat. Nos. 4,683,195 and 4,683,202 to Mullis and Mullis et al., respectively. PCR is used for the amplification and detection of low levels of specific nucleic acid sequences. PCR can be used to directly increase the concentration of the target nucleic acid sequence to a more readily detectable level. A variant of PCR is the ligase chain reaction, or LCR, which uses polynucleotides that are ligated together during each cycle. PCR can suffer from non-specific amplification of non-target sequences. Other variants exist, but none have been as widely accepted as PCR.

Hybridization techniques involve detecting the hybridization of two or more nucleic acid molecules. Such detection can be achieved in a variety of ways, including labeling the nucleic acid molecules and observing the signal generated from such a label. Traditional methods of hybridization, including Northern and Southern blotting, were developed with the use of radioactive labels which are not amenable to automation. Radioactive labels have been largely replaced by fluorescent labels in most hybridization techniques. Representative forms of other hybridization techniques include the cycling probe reaction, branched DNA, Invader™ Assay, and Hybrid Capture. However, while overcoming the problem of non-specific nucleic acid amplification associated with PCR, these techniques lack the sensitivity required for many applications, especially infectious disease diagnostics. Also, due to the use of linear amplification, many hybridization techniques can take substantial periods of time to accumulate a detectable signal.

Hybridization techniques may also be used to identify a specific sequence of nucleic acid present in a sample by using microarrays (or “bioarrays”) of known nucleic acid sequences to probe a sample. Such techniques are described in U.S. Pat. No. 6,054,270. Bioarray technologies generally involve attaching short lengths of single stranded nucleic acid to a surface, each unique short chain attached in a specific known location and then adding the sample nucleic acid and allowing sequences present in the sample to hybridize to the immobilized strands. Detection of this hybridization is then carried out by labeling, typically end labeling, of the fragments of the sample to be detected prior to the hybridization. When a sample fragment hybridizes to a specific strand on the array, a signal can be detected from the label, because the position of the hybridization reaction can be detected, and the sequence of the attached strand at that location is known, the sequence of the complementary strand from the sample that has hybridized can be deduced.

Usually the detection of hybridization is by measuring a fluorescent signal; however, methods of detection using an electrochemical detection method have been disclosed. Electrochemical detection methods, and devices used in electrochemical detection methods, are discussed in U.S. Pat. Nos. 5,776,672, 5,972,692, 6,489,160, 6,667,155, 6,670131, 6,783,935, and 6,818,109, Nakamura, et al., Drug Metab. Pharmaco., 20:3:219-225 (2005); Hashimoto and Ishimori, Lab on a Chip, 1:61-63 (2001); Hashimoto, et al., Anal. Chem., 66:21: 3830-33 (1994); Takahashi, et al., Analyst, 130:687-93 (2005); and Santos-Alvarez, et al., Anal Bioanal. Chem., 378:104-118 (2004) herein incorporated by reference. These electrochemical detection techniques may provide a result in a reduced time period compared to the fluorescent methods of hybridization detection. As discussed above; however, whether fluorescent or electrochemical, hybridization detection methods can be subject to false positives due to non-specific hybridization. Additionally, nucleic acid detection techniques requiring steps of nucleic acid extraction, isolation and purification may lengthen the time taken to achieve a result and also decrease the detection level of the test through the loss of nucleic acid molecules in the many washing steps involved in these isolation steps.

Nucleic acid detection techniques, while overcoming the potential problem of multiplexing associated with ELISA (i.e., the limited number of discriminatory signals), are restricted in use to only detecting nucleic acid. Therefore, agents such as proteins, drugs, hormones, chemical toxins, and prions, which do not contain nucleic acids, cannot be detected by these nucleic acid hybridization techniques. An ideal multiplex detection assay would combine the versatility of antibody recognition with the multiplexing capability and speed of controlled electrochemical detection of nucleic acid hybridization.

Clearly, an accurate, speedy multiplex detection assay to diagnose many different cancers is desirable. The present invention provides methods and compositions for such an assay.

### SUMMARY OF THE INVENTION

The present invention provides for the early, rapid and facile detection and/or diagnosis of cancer through the detection of cancer markers in biologic fluids including, inter alia, urine and blood. In certain embodiments, the present invention solves the problem of multiplex detection for multiple cancer markers, while eliminating the need for nucleic acid isolation/amplification and the problems associated with non-specific nucleic acid hybridization. The non-specific hybridization and low sensitivity observed in the detection methods currently known in the art are overcome by the present invention which provides novel methods that exploit, in a synergistic manner, the high sensitivity and selectivity of antibody: antigen interaction and nucleic acid hybridization.

In certain embodiments, the present invention provides for early, rapid, facile and accurate detection and/or diagnosis of cancers through the detection of cancer markers in biological fluids. The present invention combines the versatility of antibody recognition with the speed and sensitivity of electrochemical nucleic acid detection, yet eliminates the need for nucleic acid isolation/amplification and the problems associated with non-specific nucleic acid hybridization. The non-specific hybridization and low sensitivity observed in other diagnostic methods currently known in the art are overcome by nucleic acid sequences that are rationally designed to minimize non-specific hybridization, and ensure that sequence-specific hybridization is optimized.

There is a long standing and recognized need for methods of cancer detection. Because many markers identified for various cancer states are present in trace amounts (low concentrations in biological samples), traditional antibody-based techniques fail to detect them specifically or accurately, if at all. Nucleic acid based methods are not applicable and/or are ineffective for a variety of reasons including, inter alia, the common problem that the gene that encodes the marker in question may be identified or identifiable, but is not detectable or amplifiable. Alternatively, the protein markers are typically transcriptionally controlled such that the detection and/or amplification of the encoding genetic material not necessarily indicative of a cancerous state. Hence, there exists a real and long-recognized need for a methodology that facilitates the early detection of cancer in a reliable, accurate and, preferably, facile manner. The present invention overcomes the failings and shortcomings of the prior known methods.

In one embodiment of the present invention, a method of electrochemically detecting the presence of one or more cancer markers in a sample is taught. This embodiment includes the use of (1) one or more chip-associated oligos, (2) one or more capture-associated oligos that has the same sequence as or a sequence substantially similar to the respective chip-associated oligo, wherein each capture-associated oligos comprise a capture moiety specific for the particular cancer marker to be detected and, optionally, comprises a selectively activatable promoter (e.g., a T7 promoter) or other moiety to enable amplification (e.g., PCR primer site), (3) immobilized binding partners to the capture moiety, to the cancer markers or to the capture moiety/cancer marker complex, (4) a polymerase capable of interacting with said promoter to polymerize the capture-associated oligo to produce polymerization products that are complementary or substantially complementary to the chip-associated oligos and the capture-associated oligos and (5) a sample suspected of containing the cancer marker(s). The present invention also includes kits that comprise one or more of the forgoing features/elements (1)-(5).

Certain embodiments of the methods of the present invention include, not limited to the following order or requiring each step, mixing (or otherwise contacting) the sample containing the suspected target agent (i.e., a cancer marker) with the capture-associated oligos to allow the capture moiety to bind the cancer marker to form a first complex. The first complex can be selectively removed from solution (or otherwise isolated) and, in a preferred embodiment, immobilized in a reaction vessel using immobilized binding partners. The selectable promoter is preferably activated in such a manner so as to enable the polymerase to interact with the promoter and polymerize the capture-associated oligo to form sequences complementary to the chip-associated oligo (“polymerization products”). The polymerization products of the present invention can comprise, inter alia, DNA, RNA or a combination thereof. In a preferred embodiment, the polymerization product(s) are RNA sequences that are complementary (or substantially complementary) to the sequence of the chip-associated oligo.

The polymerization products (in crude, partially purified or purified form) are contacted with chip-associated oligos, where a hybridization event between the chip-associated oligos and one or more polymerization products indicates that a target agent (i.e., a cancer marker) was present in the sample. The hybridization event is detected by one or more methods including, inter alia, electrochemical detection, gel isolation, direct or indirect fluorescence detection, radioactive labels, RIA, ELISA, PCR, etc. These means, in particular the electrochemical detection, can be direct or indirect (i.e., involving the use of one or more electrochemical hybridization indicators such as, inter alia, intercalating agents, minor groove binding agents, conjugated antibodies and/or other nucleic acid binding agents).

In a preferred embodiment, the complexed capture-associated oligo is subjected to one or more rounds of one or more types of amplification. In a particularly preferred embodiment, isothermal amplification can be employed to produce the polymerization products to increase the number of single stranded nucleic acid molecules available for binding (e.g., annealing to, hybridizing with, etc.) to the chip-associated oligo, thereby enhancing the signal created through a hybridization event. In these embodiments, the capture-associated oligo can be used as a template for linear amplification, with the capture-associated oligo being preferably designed to encode a complementary (or substantially complementary) sequence to a polymerase recognition sequence at its 3′ end following the complementary (or substantially complementary) region of the chip-associated oligo. Following interaction of the cancer marker with the capture moiety, the resultant complex is isolated (e.g., via immobilization with immobilized binding partners) and contacted with a “priming” oligonucleotide—an oligonucleotide that is complementary to, inter alia, the 5′ to 3′ polymerase recognition sequence to form a double-stranded polymerase recognition site. Following annealing of the priming oligonucleotide to the capture-associated oligo, an excess of mononucleotides and the appropriate polymerase(s) can be added under conditions that promote of otherwise facilitate polymerization and linear amplification of the capture-associated oligo to form polymerization products. This polymerization reaction is preferably performed under conditions that allow for the repeated use of the template strand (e.g., the capture-associated oligo) for multiple rounds of polymerization so as to result in multiple copies of the polymerization products being formed from each such complexed capture-associated oligo. In such embodiments, the chip-associated oligo will have the same sequence (or substantially similar sequence) as the capture-associated oligo, and both will be complementary (or substantially complementary) to the polymerization product(s). In a preferred embodiment, the polymerase recognition site created by this double-stranded region is a phage-encoded RNA polymerase recognition sequence (e.g., polymerase recognition sequences for T7, T3, SP6, and the like).

In an alternative embodiment, the immobilization binding partners bind to the capture moieties that have bound the cancer markers instead of binding the unreacted capture moieties in an “antibody capture” scenario. For example, in the case where the capture moiety is an antibody, the capture-associated oligo is mixed with a sample suspected of containing the cancer marker, in this case, an antigen. In contrast to other embodiments described thus far, the resulting first mixture is then contacted with an immobilized antibody to the same target antigen, immobilizing the [oligo-antibody-cancer marker] complex by formation of an [oligo-antibody-cancer marker-antibody] complex in a second mixture. The second mixture will, typically, have a solution phase comprising the oligo-conjugated capture moiety (in this example, an antibody) that did not capture a cancer marker (in this case, an antigen) and an immobilized phase comprising the [oligo-antibody-cancer marker-antibody] complex. The solution phase can be removed from the reaction mixture by known methods including, but not limited to decanting, centrifugation, washing, etc. The immobilized [oligo-antibody-cancer marker-antibody] complex is then released (or, in some embodiments, the oligo is cleaved from the [oligo-antibody-cancer marker-antibody] complex) into solution, where the solution is transferred to, e.g., an electrochemical detection device for detection. Through this process, the only oligos present in the third mixture transferred and introduced into the electrochemical device are those polymerization products that correspond to capture-associated oligos that captured a cancer marker (in this case, an antigen) present in the sample. The immobilized binding partners can be the same capture moiety conjugated to the capture-associated oligo, or, preferably, are binding partners that recognize a different epitope of the cancer marker or can recognize the capture moiety/cancer marker complex. As in other embodiments, multiple different capture-associated oligos can be employed (so-called multiplexing), thereby allowing for the simultaneous screening and detection of multiple cancer markers from a single sample.

In certain embodiments, the polymerization product comprises RNA sequences that are complementary to the chip-associated oligos. Thus, hybrids resulting from hybridization between the chip-associated oligo and the polymerization products will be DNA:RNA (if the chip-associated oligo is DNA) or RNA:RNA (if the chip-associated oligo is RNA) duplexes. The resulting hybrids can thus be detected by an antibody reagent capable of binding to the DNA:RNA or RNA:RNA duplexes formed. A variety of protocols and reagent combinations can be employed in order to carry out the principles of the present method, and detection of the antibody reagent to hybridization duplexes can be accomplished in any convenient manner. In a preferred embodiment, the antibody reagent is labeled with a moiety such as an enzymatically active group, a fluorescer, a chromophore, a luminescer, a specifically bindable ligand, a electrochemically detectable molecule/moiety, a radioisotope or the like, with the nonradioisotopic labels being especially preferred. The labeled antibody reagent which becomes bound to resulting immobilized hybrid duplexes can be readily separated from that which does not become so bound.

Another aspect of the present invention provides capture moieties of cancer markers conjugated to oligos for use in the methods of the present invention.

### DESCRIPTION OF THE FIGURES

So that the manner in which the above recited features, advantages and objects of the present invention are attained and can be understood in detail, a more particular description of the invention, briefly summarized above, may be had by reference to the embodiments that are illustrated in the appended drawings. It is to be noted, however, that the appended drawings illustrate only certain embodiments of this invention and are therefore not to be considered limiting of its scope, for the present invention may admit to other equally effective embodiments.

In the following figures, the filled rectangles (black or white) represent nucleic acid sequences, with the white rectangles representing nucleic acids complementary to the black sequences. The striped rectangle represents the conjugation structure linking the capture-associated oligo to the capture moiety. Each capture moiety is represented by an arrow structure, and the cancer marker is represented by a hexagon. The substrate to which the immobilized binding partners are bound is represented by a circle.

FIG. 1 provides a representative overview flow diagram showing one embodiment of a method for diagnosing various cancers in accordance with the present invention. It is important to recognize that the capture moiety conjugated oligo can be pre-made and need not be made in conjunction with the practicing of the methods of the present invention.

FIG. 2 provides a flow diagram showing a method for selecting universal oligos and universal oligo sets.

FIG. 3 is a schematic diagram demonstrating the detection of a cancer marker using a immobilized binding partners for isolation of the capture-associated oligo. A: The first step is exposure of the capture-associated oligos to the sample to bind the cancer markers in the sample. B: Once the capture moiety has bound a cancer markers, the complex is exposed to immobilized binding partners for isolation. C: Following isolation of the complex, the complex is introduced to the chip-associated oligos. D: The binding of the complex to the chip-associated oligos will generate a signal in, e.g., an electrochemical detection device.

FIG. 4 is a schematic diagram illustrating an embodiment of detection of a cancer marker using immobilized binding partners for isolation of the capture-associated oligo/cancer marker complex. In this specific embodiment the immobilized binding partners bind to an epitope comprising both the capture moiety of the capture-associated oligo and the cancer marker. A: The first step is exposure of the capture-associated oligo to the sample. B: Once the capture moiety has bound its cancer marker, the complex is exposed to immobilized binding partners for isolation. C: Following isolation of the complex, it is introduced to the chip-associated oligos. D: The binding of the complex to the chip-associated oligos will generate a signal in, e.g., an electrochemical detection device.

FIG. 5 is a schematic diagram illustrating an embodiment of the use of an engineered polymerase recognition site to create multiple copies of the capture-associated oligo for more sensitive detection of cancer markers. The engineered polymerase site is represented by a dappled rectangle. A: The binding of an oligonucleotide complementary to the single-stranded polymerase recognition sequence of the capture-associated oligo provides a double-stranded polymerase recognition site. B: The complex is reacted with appropriate nucleotides and a polymerase to provide polymerization products. C: The reactions are carried out to create multiple copies of the capture-associated oligo via linear amplification.

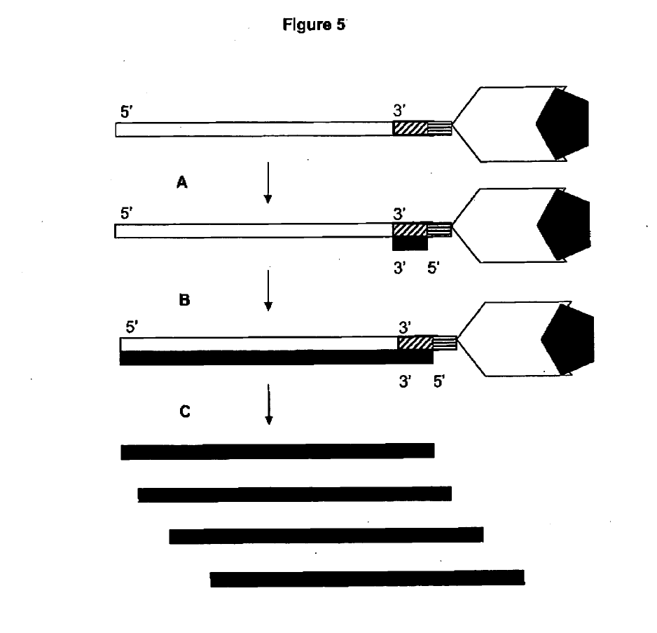

FIG. 6 is a schematic diagram illustrating an embodiment of the use of an engineered capture-associated oligo comprising a restriction endonuclease site and a polymerase recognition site. The engineered restriction site is represented by a grey rectangle, and the engineered polymerase site is represented by a dappled rectangle. A: The binding of an oligonucleotide complementary to the encoded single-stranded polymerase recognition sequence of the capture-associated oligo provides a double-stranded polymerase recognition site and a restriction endonuclease cleavage site. B: The complex is reacted with the appropriate restriction endonuclease to remove the capture moiety-cancer marker complex. C: The cleaved capture-associated oligo is reacted with the appropriate nucleotides and polymerase to provide creation of polymerization products. D: The reactions are carried out to create multiple copies of polymerization products via linear amplification.

FIG. 6b is a schematic diagram illustrating an embodiment of the use of an engineered capture-associated oligo comprising a restriction endonuclease site and a polymerase recognition site. The engineered restriction site is represented by a grey rectangle, and the engineered polymerase site is represented by a dappled rectangle. The striped rectangle represents the conjugation structure linking the capture-associated oligo to the capture moiety. A: The binding of the two oligonucleotides complementary to the encoded single-stranded polymerase recognition sequence and restriction endonuclease cleavage sequence portions of the capture-associated oligo provides a double-stranded polymerase recognition site and a restriction endonuclease cleavage site. B: The complex is reacted with the appropriate restriction endonuclease to remove the capture moiety-cancer marker complex from the capture-associated oligo. C: The cleaved capture-associated oligo is reacted with the appropriate nucleotides and polymerase to provide polymerization products complementary to the capture-agent associated nucleic acid. D: The reactions are carried out to create multiple copies of the capture-associated oligos via linear amplification.

FIG. 7 is a schematic diagram illustrating an embodiment of the combination of isolation using immobilized binding partners that bind to the cancer marker and polymerase amplification techniques. A: The first step is exposure of the capture-associated oligo to the sample for binding of the cancer markers in the sample. B: Once the capture moiety has bound its cancer marker, the complex is exposed to immobilized binding partners for isolation. C: The binding of an oligonucleotide complementary to the encoded single stranded polymerase recognition sequence of the capture-associated oligo provides a double-stranded polymerase recognition site. D: The complex is reacted with the appropriate nucleotides and polymerase to provide polymerization products complementary to the capture-agent associated nucleic acid. E: The reactions are carried out to create multiple copies of the capture-associated oligo via linear amplification. F: The polymerization products are introduced to the chip-associated oligo. D: The binding of the polymerization products to the chip-associated oligos will generate a signal in, e.g., an electrochemical detection device.

FIG. 8 is a schematic diagram illustrating an embodiment of the combination of isolation using immobilized binding partners that bind to a capture moiety-cancer marker epitope, restriction endonuclease cleavage of the capture moiety-cancer marker complex from the capture-associated oligo, and a polymerase amplification techniques. A: The first step is exposure of the capture-associated oligo to the sample. B: Once the capture moiety has bound its cancer marker, the complex is exposed to immobilized binding partners for isolation. C: The binding of an oligonucleotide complementary to the encoded single-stranded polymerase recognition sequence portion of the capture-associated oligo provides a double-stranded polymerase recognition site and a restriction endonuclease cleavage site. D: The complex is reacted with the appropriate restriction endonuclease to remove the capture moiety-cancer marker complex from the capture-associated oligo. E: The cleaved nucleic acid is reacted with the appropriate nucleotides and polymerase to create polymerization products complementary to the capture-associated oligo. F: The reactions are carried out to create multiple copies of the capture-associated oligos via linear amplification. G: The polymerization products are introduced to the chip-associated oligos. H: The binding of the polymerization products to the complementary chip-associated oligos will generate a signal in, e.g., an electrochemical detection device.

FIG. 8b is a schematic diagram illustrating an embodiment of the combination of isolation using immobilized binding partners that bind to a capture moiety-cancer marker epitope, restriction endonuclease cleavage of the capture moiety-target agent complex from the capture-associated oligo, and polymerase amplification techniques. A: The first step is exposure of the capture-associated oligo to the sample. B. Once the capture moiety has bound its cancer marker, the complex is exposed to immobilized binding partners for isolation. C: The binding of the two oligonucleotides complementary to the encoded single-stranded polymerase recognition sequence and restriction endonuclease cleavage sequence portions of the capture-associated oligo provides a double-stranded polymerase recognition site and a restriction endonuclease cleavage site. D: The complex is reacted with the appropriate restriction endonuclease to remove the capture moiety-cancer marker complex from the capture-associated oligo. E: The cleaved capture-associated oligo is reacted with the appropriate nucleotides and polymerase to create polymerization products complementary to the capture-associated oligo. F: The reactions are carried out to create multiple copies of the capture-associated oligo via linear amplification. G: The polymerization products are introduced to the chip-associated oligos. H: The binding of the polymerization products to the chip-associated oligos will generate a signal in, e.g., an electrochemical detection device.

### DEFINITIONS

The terms used herein are intended to have the plain and ordinary meaning as understood by those of ordinary skill in the art unless otherwise specifically defined. The following definitions are intended to aid the reader in understanding the present invention, but are not intended to vary or otherwise limit the meaning of such terms unless specifically indicated.

The term “nucleic acid molecules”, “oligos”, “oligonucleotides” or “polynucleotides” as used herein refers to linear oligomers of natural or modified nucleic acid monomers or linkages, including, inter alia, deoxyribonucleotides, ribonucleotides, anomeric forms thereof, peptide nucleic acid monomers (PNAs), locked nucleotide acid monomers (LNA), and the like, each of which may be capable of specifically binding to a single stranded polynucleotide by way of a regular pattern of monomer-to-monomer interactions, such as Watson-Crick type of base pairing, base stacking, Hoogsteen or reverse Hoogsteen types of base pairing, or the like. Typically monomers are linked by phosphodiester bonds or analogs thereof to form oligonucleotides ranging in size from a few monomeric units, e.g., 8-12, larger numbers of monomeric units, e.g., 100-200 ad even larger, e.g., 100-9000. Suitable nucleic acid molecules may be prepared by the phosphoramidite method described originally by Beaucage and Carruthers (Tetrahedron Lett., 22, 1859-1862, 1981), or by the triester method described originally by Matteucci, et al. (J. Am. Chem. Soc., 103, 3185, 1981), both incorporated herein by reference, or by other chemical methods such as using a commercial automated oligonucleotide synthesizer and/or other known methodologies. “Capture-associated oligos” refers to oligos that are conjugated to or otherwise associated with a capture moiety. “Chip-associated oligos” refers to oligos that are immobilized on or otherwise associated with a substrate but need not be limited to immobilization on a “chip.” In certain embodiments, the chip-associated oligo is immobilized on surfaces other than chips including, inter alia, reaction vessels, filters, membranes, beads, and the like. In other embodiments, the “chip-associated” oligo is not immobilized but is captured together with its complementary strand, for example by use of an antibody that specifically recognizes a RNA:DNA hybrid duplex (discussed further, infra). In certain preferred embodiments the chip-associated oligo is immobilized on or otherwise associated with a chip.

The terms “complementary” or “complementarily” are used in reference to nucleic acid molecules (i.e., a sequence of nucleotides) that are related by base-pairing rules. Complementary nucleotides are, generally, A and T (or A and U), or C and G. Two single stranded RNA or DNA molecules are said to be substantially complementary when the nucleotides of one strand, optimally aligned and with appropriate nucleotide insertions or deletions, pair with at least about 80% of the nucleotides of the other strand, usually at least about 90% to 95%, and more preferably from about 98 to 100%. Alternatively, substantial complementarily exists when an RNA or DNA strand will hybridize under selective hybridization conditions to its complement. Selective hybridization conditions include, but are not limited to, stringent hybridization conditions. Selective hybridization typically refers to embodiments wherein there is at least about 65% complementarily over a stretch of at least 14 to 25 nucleotides, preferably at least about 75%, more preferably at least about 90% complementarily. See, M. Kanehisa, Nucleic Acids Res. 12, 203 (1984), incorporated herein by reference. For shorter nucleotide sequences selective hybridization occurs when there is at least about 65% complementarily over a stretch of at least 8 to 12 nucleotides, preferably at least about 75%, more preferably at least about 90% complementarily. Stringent hybridization conditions will typically include salt concentrations of less than about 1 M, more preferably less than about 500 mM and preferably less than about 200 mM. Hybridization temperatures can be as low as −80° C., preferably greater than about 5° C., and are preferably lower than about 30° C. However, longer fragments may require elevated hybridization temperatures for specific hybridization. Hybridization temperatures are generally at least about 1° C. to 20° C. lower than melting temperatures (Tₘ), which is defined below, preferably about 1° C. to about 12° C. lower than melting temperature, and more preferably about 2° C. to about 8° C. lower than melting temperature.

The term “universal oligo” generally refers to one oligonucleotide of a complementary oligonucleotide pair, where each oligonucleotide in the pair has been rationally designed to have low complementarily to all sequences that may be present in a sample. For example, in a blood sample for diagnosis of hepatitis in a human, a universal oligo would be one with low complementarily to human genomic sequences, genomic sequences from hepatitis viruses, as well as genomic sequences of organisms that associate with humans (e.g., human gut flora). For a soil sample, a universal oligo would be one with minimal complementarily to genomic sequences from, e.g., soil flora and fauna. A “universal oligo set” is a set of two or more universal oligo pairs where each oligo in the set has low complementarily to every other universal oligo in the set, with the exception of its complement. A “universal oligo chip” is an array of two or more universal oligos—each from a different universal oligo pair—that are immobilized at a known location on a surface such as glass, plastic, nylon, silicon, etc. The term “capture-associated universal oligo” refers to the oligo of a universal oligo pair that is associated with a capture moiety. The term “chip-associated universal oligo” refers to the oligo of a universal oligo pair that is immobilized on or otherwise associated with a substrate but need not be limited to immobilization on a “chip.” In certain embodiments, the chip-associated oligo is immobilized on surfaces other than chips including, inter alia, reaction vessels, filters, membranes, beads, and the like. In other embodiments, the “chip-associated” oligo is not immobilized but is captured together with its complementary strand, for example by use of an antibody that specifically recognizes a RNA:DNA hybrid duplex (discussed further, infra). In certain preferred embodiments the chip-associated oligo is immobilized on or otherwise associated with a chip.

In certain embodiments of the present invention, capture-associated universal oligos and chip-associated universal oligos are complementary or substantially complementary; however, in embodiments where linear amplification of the capture-associated universal oligo is employed (as described in detail infra), the capture-associated universal oligos and the chip-associated universal oligos are complementary or substantially complementary and the amplification products derived from the capture-associated universal oligo are complementary to the capture-associated universal oligos and the chip-associated universal oligos.

The term “capture” is intended to convey any association, including, inter alia, conjugation, irreversible binding, reversible binding, covalent binding, intercalation, non-covalent binding, etc. A “capture moiety” refers to a portion of a molecule that can be used to preferentially associate with or bind to and separate a molecule of interest (a “target agent”) present in or potentially present in a sample. The term “capture moiety” as used herein refers to any molecule, natural, synthetic, or recombinantly produced, with the ability to bind to the target agent in any of the methods of the present invention. The binding affinity of the capture moiety must be sufficient to allow collection of the target agent from a sample. Suitable capture moieties include, inter alia, antibodies, antigen-binding regions of antibodies, antigens, epitopes, cell receptor ligands, such as peptide growth factors (see, e.g., Pigott and Power (1993), The Adhesion Molecule Facts Book (Academic Press New York); and Receptor Ligand Interactions: A Practical Approach, Rickwood and Hames (series editors) Hulme (ed.) (IRL Press at Oxford Press NY)). Similarly capture moieties may also include but are not limited to toxins, venoms, intracellular receptors (e.g., receptors that mediate the effects of various small ligands, including steroids, hormones, retinoids and vitamin D, peptides), drugs (e.g., opiates, steroids, etc.), lectins, sugars, oligosaccharides, other proteins, and phospholipids. Persons of ordinary skill in the art readily will appreciate that other and varied capture moieties based upon other molecular interactions than those listed above are well described in the literature and may also serve as capture moieties.

By “preferentially binds” it is meant that a capture moiety is designed to be at least 5-20 times or more, preferably 20-50 times or more, more preferably 50-100 times or more, and even more preferably 100-1000 times or more likely to bind to the intended target agent than to other molecules in a biological solution. In the embodiment where the capture moiety is comprised of antibody, the binding affinity may be due to (1) a single monoclonal antibody (i.e., large numbers of one kind of antibody) or (2) a plurality of different monoclonal antibodies (e.g., large numbers of each of five different monoclonal antibodies) or (3) large numbers of polyclonal antibodies. It is also possible to use combinations of (1)-(3). The four-fold differential in binding affinity may be accomplished by using several different antibodies as per (1)-(3) above and as such some of the antibodies in a mixture could have less than a four fold difference. For purposes of the invention an indication that no binding occurs means that the equilibrium or affinity constant Kₐ is 10⁶ l/mole or less. Antibodies may be designed to maximize binding to the intended antigen by designing the peptides to specific epitopes that are more accessible to binding, as can be predicted by one skilled in the art.

A “target agent” is intended to refer to a target moiety in a sample that is to be captured through preferential binding with the capture moiety; here, a cancer marker. For example, in the case where the capture moiety is an antibody, the target agent will be any molecule which contains the epitope against which the antibody is generated. Where the capture moiety is a protein used for detection of an antibody, the antibody itself is the target agent. Target agents include organic and inorganic molecules, including biomolecules. In some embodiments, the target agent may be an environmental pollutant (including pesticides, insecticides, toxins, etc.); a chemical (including solvents, organic materials, etc.); therapeutic molecules (including therapeutic and abused drugs, antibiotics, etc.); biomolecules (including hormones, cytokines, proteins, lipids, carbohydrates, cellular membrane antigens and receptors (neural, hormonal, nutrient, and cell surface receptors) or their ligands, etc); whole cells (including procaryotic (such as pathogenic bacteria) and eucaryotic cells, including mammalian tumor cells); viruses (including retroviruses, herpesviruses, adenoviruses, lentiviruses, etc.); and spores; etc. In the present invention, cancer markers are detected. Cancer markers include virtually any biological molecule such as antibodies, antigens, hormones, cytokines, proteins, lipids, carbohydrates, cellular membrane antigens and receptors, metabolites and the like. A representative non-limiting listing of suitable cancer markers includes, inter alia: Prostate-specific antigen (PSA), Prostatic acid phosphatase (PAP), CA 125, Carcinoembryonic antigen (CEA), Alpha-fetoprotein (AFP), Human chorionic, gonadotropin (HCG), CA 19-9, CA 15-3, CA 27-29, lactate dehydrogenase (LDH) Neuron-specific enolase (NSE), Bcl-2 proto-oncogene, BCA-225, c-Met, CA 15-3 Cathepsin D, Cytokeratin (HMW), Human epithelial cadherin (E-cadherin, uvomorulin) E2F-1, EGF receptor, EphB4 receptor and ephrin-B2 ligand, Estrogen receptor and progesterone receptor, EZH2, also known as ENX-1, FHIT (Fragile Histidine Triad) GCDFP-15, HER2 (c-erbB-2), HER4 (c-erbB-4), Ki-67, Metallothioneins, MTA1 Mucin 1, NY-ESO-1, a member of the CT (cancer/testis) family, p53 (mutant), PRL-3 Trefoil peptide pS2, Retinoblastoma gene (Rb), SKP2 (S-phase Kinase-associated Protein 2), Smad 3, STAT3 (Signal Transducers and Activators of Transcription3), TAG 72 (CA 72.4), Tenascin, Tissue inhibitors of metalloproteinase (TIMPs), Topo II-alpha, Urokinase-type plasminogen activator receptor (uPAR), Platelet-derived endothelial cell growth factor (PD-ECGF), Melanocortin-1 receptor (MCI R) mutant variants, AMACR (Alpha-methylacyl-CoA-racemase (P504S)), p63, CD117 (c-Kit) receptor, COX-2, Ezrin FLI-1 protein, Galectin-3, Galectin-7, Glutaminyl cyclase, MAGE-A1/2, MAGE-A3/4, MAGE-A12, Mesothelin 1, Prostate and testis expressed protein (PATE), Kallikriens Pax5, PDEF (prostate-derived Ets factor), PSMA (prostate specific membrane antigen) Serum Receptor-Binding Cancer Antigen (RCAS1), Tyrosinase, Prolactin (PRL) and its receptor (PRLr), Beta Catenin, Matrix metalloproteinase-7, Claudin-3, Claudin-4, Cyclin E, O6-methylguanine-DNA methyltransferase (MGMT), Human placental alkaline, phosphatase, Calretinin, CD15, Thyroid Transcription Factor 1, Wilm's tumor 1 (WT-1) MART-1 (Melan-A), Nucleophosmin/B23 (NPM), p21 (Waf1/Cip1), p16 (INK4a) Cyclin-dependent kinase inhibitor p27 (kip1), Pituitary Tumor Transforming Gene-1 Glycosylated eosinophil-derived neurotoxin & osteopontin, Fibrinopeptide A, Uridine phosphorylase, Modified urinary nucleosides, Ras, Myc, CDKN2A, Phosphatase and tensin homolog deleted from chr. 10, C-erbB-2, Vascular endothelial growth factor receptor.

The term “sample” in the present specification and claims is intended to be used in its broadest sense and can be, by non-limiting example, any sample that is suspected of containing the target agents (e.g., cancer markers) to be detected. It is intended that the term “sample” mean specimens or cultures taken from a human. In a preferred embodiment, the samples are from a human. Specific, but non-limiting examples include, inter alia: urine or other urinary or renal discharge, blood and/or blood components, sputum, tissue, spinal fluid, serum, nasal swabs or discharge, throat swabs or discharge, wound discharge, ocular fluid, bile, stool or other rectal discharge, proteinaceous material, mucous, semen and or seminal fluids or components thereof, vaginal fluids and/or discharge and/or components thereof. Biological samples may include, but are not limited to, sputum, amniotic fluid, whole blood, blood cells (e.g., white cells), blood serum, urine, semen, peritoneal fluid, pleural fluid, pericardial fluid, feces, ascetic fluid, spinal fluid, synovial fluid, tissue specimens or fine needle biopsy samples, and tissue homogenates. Samples may also include sections of tissues such as frozen sections taken for histological purposes. These examples are not to be construed as limiting the sample types applicable to the present invention. Those with skill in the art would appreciate and understand the particular type of sample required for the detection of particular target antigens and an appropriate procedure for sample preparation (see, e.g., Tietz Textbook of Clinical Chemistry and Molecular Diagnostics, 4^{th} Ed., Chapter 2, Burtis, C. Ashwood E. and Bruns, D, eds. (2006); Chemical Weapons Convention Chemicals Analysis: Sample Collection, Preparation and Analytical Methods, Mesilaakso, M., ed., (2005); Pawliszyn, J., Sampling and Sample Preparation for Field and Laboratory, (2002); Venkatesh Iyengar, G., et al., Element Analysis of Biological Samples: Principles and Practices (1998); Drielak, S., Hot Zone Forensics: Chemical, Biological, and Radiological Evidence Collection (2004); Wells, D., High Throughput Bioanalytical Sample Preparation (Progress in Pharmaceutical and Biomedical Analysis) (2002)), each of which is incorporated by reference.

The term “antibody” as used herein is intended to refer to an entire immunoglobulin or antibody or any functional fragment of an immunoglobulin molecule which is capable of specific binding an antigen. Antibody as used herein is meant to include the entire antibody as well as any antibody fragments capable of binding the antigen or antigenic fragment of interest. Examples of such peptides include complete antibody molecules, antibody fragments, such as Fab, F(ab′)₂, CDRS, V_{L}, V_{H}, and any other portion of an antibody which is capable of specifically binding to an antigen. An IgG antibody molecule is composed of two light chains linked by disulfide bonds to two heavy chains. The two heavy chains are, in turn, linked to one another by disulfide bonds in an area known as the hinge region of the antibody. A single IgG molecule typically has a molecular weight of approximately 150-160 kD and containing two antigen binding sites. An F(ab′)₂ fragment lacks the C-terminal portion of the heavy chain constant region, and has a molecular weight of approximately 110 kD. It retains the two antigen binding sites and the interchain disulfide bonds in the hinge region, but it does not have the effector functions of an intact IgG molecule. An F(ab′)₂ fragment may be obtained from an IgG molecule by proteolytic digestion with pepsin at pH 3.0-3.5 using standard methods such as those described in Harlow and Lane, supra. Preferred antibodies for assays of the invention are immunoreactive or immunospecific for, and therefore specifically and selectively bind to, a cancer marker. A “purified antibody” refers to that which is sufficiently free of other proteins, carbohydrates, and lipids.

A substance is commonly said to be present in “excess” or “molar excess” relative to another component if that component is present at a higher molar concentration than the other component. Often, when present in excess, the component will be present in at least a 10-fold molar excess and commonly at 100-1,000,000 fold or greater molar excess. Those of skill in the art would appreciate and understand the particular degree or amount of excess preferred for any particular reaction or reaction conditions. Such excess is often empirically determined and/or optimized for a particular reaction or reaction conditions. The specific degree of excess preferred for any particular diagnostic will be readily understood by those of ordinary skill in the art.

The term “reacted nucleic acid molecules” or “reacted molecules” is used in reference to those nucleic acid molecules that have a conjugated capture moiety for a particular target agent, where the target agent is present in the sample, and the corresponding capture moiety has bound to the target agent. The term “unreacted nucleic acid molecules” or “unreacted molecules” is used in reference to those nucleic acid molecules that have a conjugated capture moiety for a particular target agent, but the target agent was not present in the sample—or was present in an amount less than the capture moiety—and the corresponding capture moiety has not bound the particular target agent.

The term “capture reaction” may be used in reference to the mixing/contacting of the nucleic acid molecules conjugated to a capture moiety and the sample under conditions that allow the capture moiety to attach to, bind or otherwise associate with target agent in the sample.

The term “melting temperature” or Tm is commonly defined as the temperature at which a population of double-stranded nucleic acid molecules becomes half dissociated into single strands. The equation for calculating the Tm of nucleic acids is well known in the art. As indicated by standard references, a simple estimate of the Tm value may be calculated by the equation: Tm=81.5+16.6(log₁₀[Na⁺])0.41(%[G+C])−675/n−1.0m, when a nucleic acid is in aqueous solution having cation concentrations of 0.5 M, or less, the (G+C) content is between 30% and 70%, n is the number of bases, and m is the percentage of base pair mismatches (see e.g., Sambrook J et al., “Molecular Cloning, A Laboratory Manual, 3^{rd} Edition, Cold Spring Harbor Laboratory Press (2001)). Other references include more sophisticated computations, which take combinations of structural characteristics as well as sequence characteristics and reaction conditions into account for the calculation of a particular Tm.

The term “matrix” means any surface. Suitable matrices include those made from, inter alia, glass, nylon, polymethylacrylamide, polystyrene, polyvinyl chloride, latex, chemically modified plastic, cellulose, rubber, red blood cells, polymeric materials or biological materials.

A “restriction endonuclease” is any enzyme capable of recognizing a specific sequence (the “restriction site”) on a double- or, preferably, single-stranded polynucleotide and cleaving the polynucleotide at or near the site. Examples of site-specific restriction endonucleases are available in the 2006 New England Biolabs, Inc. Catalog, including the 2006 New Products Catalog Supplement, which is incorporated herein by reference. The term “moiety that is capable of creating a signal” encompasses virtually any of the signal generating systems used in the prior art and any system to be developed in the future. It comprises a moiety which generates a signal itself, e.g., a dye, a radioactive molecule, a chemiluminescent material, a fluorescent material or a phosphorescent material, or a moiety which upon further reaction or manipulation will give rise to a signal, e.g., an enzyme linked system. Suitable enzymes that can be utilized to create a signal are essentially any enzyme that is capable of generating a signal when treated with a suitable reagent. Preferred enzymes are horseradish peroxidase, alkaline phosphatase, glucose oxidase, peroxidase, acid phosphatase and beta-galactosidase. Such enzymes are preferred because they are very stable, yet highly reactive. Another method in which the target genetic material can be detected is a method in which each single stranded polynucleotide segment has a label, and a when a double hybrid is formed, the combination of the labels from each single stranded polynucleotide segment creates a signal; i.e., neither label of each polynucleotide alone is capable of creating a signal. In this system it is preferred that each of the two labels be attached, either covalently or via complex formation, at one end of each single stranded polynucleotide segment where when the hybrid is formed, the labels are proximate one another. Thus, in a preferred embodiment, the first label is attached in the three prime terminal position of one single stranded polynucleotide segment and the second label is attached at the five prime terminal position of the other single stranded polynucleotide segment. In a more preferred embodiment the label of each polynucleotide is capable of forming a complex, thereby increasing the proximity of the two labels and resulting in a stronger signal. Such affinity or complex formation can be naturally occurring, e.g., where an apoenzyme is one label and the apoenzyme's cofactor is the other label. In this system a signal can be created by adding a suitable reagent, but such signal is only created if the apoenzyme and its cofactor form a complex. Alternatively, the affinity or complex can be artificially created. For example, one label can be a chemiluminescent catalyst and the other label can be an absorber/emitter moiety. The oligonucleotides hybridize to each other placing the chemiluminescent catalyst and absorber/emitter moiety in proximity to produce a detectable signal. These methods can be carried out as described, by non-limiting example, in European Patent Application Publication Number 0 070 685, published Jan. 26, 1983, the disclosure of which is incorporated herein. Each of the ligand and receptors disclosed hereinabove can be utilized to create the artificial affinity.

It should be understood by those skilled in the art that terms such as “target”, “agent”, “moiety”, “antigen”, “antibody”, “molecule” and the like should be interpreted in the context in which they appear, and should be given the broadest interpretation possible unless specifically indicated.

### DETAILED DESCRIPTION OF THE INVENTION

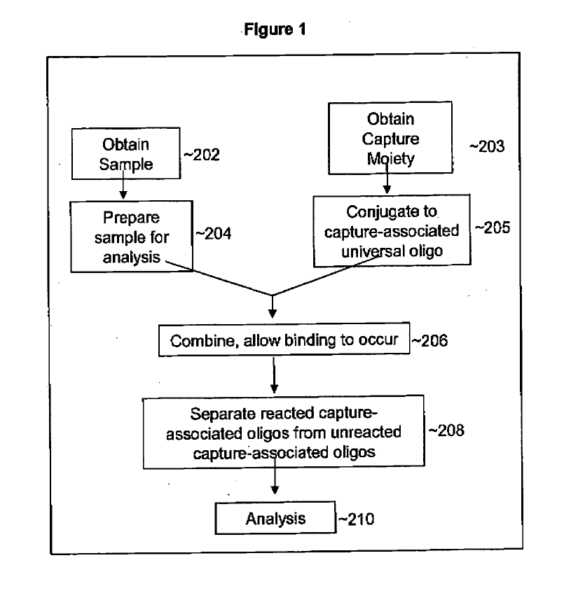

The present invention relates to compounds and methods of use for early detection of cancer markers to detect and/or diagnose cancer. One embodiment of the present invention is outlined in representative overview in FIG. 1. FIG. 1 illustrates a method 200, comprising the steps of obtaining a sample suspected of containing cancer markers, in this case, antigens (202). The sample may be blood, urine, or sputum, for example. The sample is prepared for analysis (204). One or more antibodies to the cancer marker antigens are obtained (203), and are conjugated to the one or more antibodies to capture-associated universal oligos (205). The prepared sample and the conjugated universal oligos are then combined allowing binding to occur (206), the reacted capture-associated oligos from unreacted universal oligos are separated (208), and the reacted universal oligos, if any, are analyzed (210).

Sample Processing

As seen in FIG. 1, in certain embodiments, an initial step in the methods of the present invention involves obtaining and processing a biological sample containing cancer markers antigens from a patient. Biological samples may include, but are not limited to, sputum, amniotic fluid, whole blood, blood cells (e.g., white cells), blood serum, urine, semen, peritoneal fluid, pleural fluid, pericardial fluid, feces, ascetic fluid, spinal fluid, synovial fluid, tissue or fine needle biopsy samples, and tissue homogenates. Samples may also include sections of tissues such as frozen sections taken for histological purposes.

Sample collection and preparation techniques are well known in the art (see, e.g., Tietz Textbook of Clinical Chemistry and Molecular Diagnostics, 4^{th} Ed., Chapter 2, Burtis, C. Ashwood E. and Bruns, D, eds. (2006)). In general, blood for analysis may be obtained from veins, arteries or capillaries. Venous blood is usually the specimen of choice and venipuncture is the method for obtaining this specimen. Generally, whole blood, as opposed to serum, is preferred for the present invention, as whole blood contains greater total protein. An anticoagulant must be added to the specimen during the collection procedure. A number of anticoagulants are used including heparin, EDTA, sodium fluoride, citrate, oxalate, and iodoacetate. Sputum and nasal discharge are collected directly, most commonly by swabs. A sterile Dacron® or rayon swab with a plastic shaft is preferred because calcium alginate swabs or swabs with wooden sticks may contain substances that interfere with the reactions involved in diagnosis. After collection, the swab is stored in an airtight plastic container or, preferably, immersed in liquid, such as phosphate-buffered saline or other transport medium.

Capture Moieties

The capture moieties may be any ligand associated with the cancer marker(s) to be detected. In certain embodiments, the capture moieties are antibodies, preferably monoclonal antibodies, to the cancer markers. Such capture moieties may be obtained commercially, or may be generated de novo. Table 1 comprises a exemplary list of cancer markers.

Monoclonal antibodies include a natural monoclonal antibody prepared by immunizing mammals such as mice, rats, hamsters, guinea pigs or rabbits with a cancer-associated antigen (including natural, recombinant, and chemically synthesized proteins, cell culture supernatant), or another immunogenic cancer-associated compound, or a portion thereof; a chimeric antibody or a humanized antibody produced by recombinant technology; or a human monoclonal antibody, for example, obtained by using human antibody-producing transgenic animals. Monoclonal antibodies include those having any one of the isotypes of IgG, IgM, IgA (IgA1 and IgA2), IgD, or IgE. IgG (IgG1, IgG2, IgG3, and IgG4, preferably IgG2 or IgG4) or IgM is preferable.

Polyclonal antibodies or monoclonal antibodies can be produced by known methods. Typically, mammals, preferably, mice, rats, hamsters, guinea pigs, rabbits, cats, dogs, pigs, goats, horses, or cows, or more preferably, mice, rats, hamsters, guinea pigs, or rabbits, are immunized with a cancer marker along with Freund's adjuvant, if necessary. In addition, transgenic animals may be generated so as to produce an antibody derived from another animal species, such as a human antibody-producing transgenic mouse.

Specifically, a monoclonal antibody can be produced in the following manner by methods well known in the art (see, e.g., Cellular and Molecular Immunology, 5^{th} Ed., Abbas, A. and Lichtman, A. eds. (2005)). Immunizations are accomplished by introducing a chosen cancer marker once or several times, subcutaneously, intramuscularly, intravenously, through the footpad, or intraperitoneally, into non-human mammals. Usually, immunizations are performed once to four times every one to fourteen days after the first immunization. Antibody-producing cells are obtained from the mammal in about one to five days after the last immunization. The times and interval of the immunizations can be altered in accordance with the properties of the immunogen used.

Hybridomas that secrete a monoclonal antibody can be prepared, inter alia, by the method of Kohler and Milstein (Nature, Vol. 256, p. 495-97(1975)) and by any other known methods or modified methods known in the art. Hybridomas are prepared by fusing the antibody-producing cells obtained from the spleen, lymph node, bone marrow, or tonsil from the non-human mammal immunized as mentioned above with mammal-derived myelomas that have no autoantibody-producing ability. For example, mouse-derived myelomas P3/X63-AG8.653 (653, ATCC No. CRL1580), P3/NSI/1-Ag4-1 (NS-1), P3/X63-Ag8.U1 (P3U1), SP2/0-Ag14 (Sp2/0, Sp2), PAI, F0, or BW5147; rat-derived myelomas 210RCY3-Ag.2.3; or human-derived myelomas U-266AR1, GM1500-6TG-A1-2, UC729-6, CEM-AGR, D1R11, or CEM-T15 can be used as a myelomas for the cell fusion. Monoclonal antibody producing cells (i.e., the hybridomas) can be screened by cultivating the cells, for example, in microtiter plates, and by measuring the reactivity of the culture supernatant by using the immunogen used for the immunization in an enzyme immunoassay such as an ELISA. The monoclonal antibodies may be produced from hybridomas by cultivating the hybridomas in vitro or in vivo such as in ascites of mice, rats, guinea pigs, hamsters, or rabbits, preferably mice or rats, and isolating the antibodies from the resulting culture supernatant or ascites fluid. In addition, monoclonal antibodies may be obtained in a large quantity by cloning a gene encoding a monoclonal antibody from a hybridoma or recombinant monoclonal antibody producing cell, generating transgenic animals such as cows, goats, sheep, or pigs in which the gene encoding the monoclonal antibody is integrated using transgenic animal generating techniques, and recovering the monoclonal antibody from the milk of the transgenic animals (see, e.g., Nikkei Science, No. 4, pp. 78-84 (1997)). Cultivating hybridomas in vitro typically is performed by using known nutrient media or nutrient media derived from known basal media. Examples of basal media are low calcium concentration media such as Ham F12 medium, MCDB153 medium, or low calcium concentration MEM medium, and high calcium concentration media such as MCDB104 medium, MEM medium, D-MEM medium, RPMI1640 medium, ASF104 medium, or RD medium. The basal media may also contain, for example, sera, hormones, cytokines, and/or various inorganic or organic substances known in the art.

Monoclonal antibodies can be, inter alia, isolated and purified from the culture supernatant or ascites mentioned above by saturated ammonium sulfate precipitation, euglobulin precipitation, the caproic acid or caprylic acid method, ion exchange chromatography (DEAE or DE52), thiophilic resin (Clontech®), by affinity chromatography using anti-immunoglobulin column or protein A or protein G columns, or by other methods known in the art. By using the above-mentioned methods, it is possible to immunize non-human mammals, prepare and screen hybridomas producing the antibodies, and prepare the human monoclonal antibody in large quantities (see, e.g., Nature Genetics, Vol. 7, p. 13-21, 1994; Nature Genetics, Vol. 15, p. 146-156, 1997; Published Japanese Translation of PCT International Publication No. Hei 4-504365; Published Japanese Translation of PCT International Publication No. Hei7-509137; Nikkei Science, June edition, p. 40-50, 1995; WO94/25585; Nature, Vol. 368, p. 856-859, 1994; Published Japanese Translation of PCT International Publication No. Hei 6-500233, etc.).

Monoclonal antibodies also include an antibody that comprises the heavy chain and/or the light chain in which either or both of the chains have deletions, substitutions or additions of one or several amino acids in the sequences thereof; several amino acids as referred to here means multiple amino acid residues, specifically means one to ten amino acid residues, preferably one to five amino acid residues. Such a partial modification of amino acid sequence (deletion, substitution, insertion, and addition), can be introduced into the antibody by partially modifying the nucleotide sequence encoding the amino acid sequence. The partial modification of the nucleotide sequence can be performed by the usual method of site-specific mutagenesis (see, e.g., PNAS USA, Vol. 81, p. 5662-5666 (1984)) or other methods known in the art.

An “antibody” of the present invention includes a portion of an antibody as well, including F(ab′)₂, Fab′, Fab, Fv (variable fragment of antibody), sFv, dsFv (disulfide stabilized Fv), or dAb (single domain antibody). F(ab′)₂ and Fab′ can be produced by digesting an antibody near the disulfide bonds existing between the hinge regions in each of the two H chains with a protease such as pepsin and papain, generating an antibody fragment. For example, papain cleaves IgG upstream of the disulfide bonds existing between the hinge regions in each of the two H chains to generate two homologous antibody fragments in which an L chain composed of V_{L} (L chain variable region) and C_{L} (L chain constant region), and an H chain fragment composed of V_{H} (H chain variable region) and C_{H}γ1 (gamma1 region in the constant region of H chain) are connected at their C terminal regions through a disulfide bond. Each of these two homologous antibody fragments is called Fab′. Pepsin also cleaves IgG downstream of the disulfide bonds existing between the hinge regions in each of the two H chains to generate an antibody fragment slightly larger than the fragment in which the two above-mentioned Fab′ are connected at the hinge region. This antibody fragment is called F(ab′)₂.

Antibodies may be characterized by an immunoassay such as the single antibody solid phase method, two-antibody liquid phase method, two-antibody solid phase method, sandwich method, enzyme multiplied immunoassay technique (EMIT method), enzyme channeling immunoassay, enzyme modulator mediated enzyme immunoassay (EMMIA), enzyme inhibitor immunoassay, immuno-enzymometric assay, enzyme-enhanced immunoassay or proximal linkage immunoassay, all of which are described, inter alia, in Enzyme Immunoassay, 3rd Ed., Eiji Ishikawa et al., and Igakushoin eds., (1987)); or, for example, the one-pot method which is described in JP-B Hei 2-39747. However, from the standpoint of simplicity of operation and/or economical advantage, and especially when considering the clinical applicability, the sandwich method, the one pot method, the single antibody solid phase method or the two-antibody solid phase method are preferred. Most preferable is the sandwich method using a labeled antibody prepared by labeling an antibody generated with an enzyme or biotin and using an antibody-immobilized insoluble carrier prepared by immobilizing the monoclonal antibody on a multi-well microplate.

### Universal Oligo Sets and Universal Oligo Chips

The universal oligos of the present invention are oligonucleotides from a complementary or substantially complementary oligonucleotide pair, where each oligo in the pair has been rationally designed to have low complementarily to sequences that may be present in a given sample. A “universal oligo set” is a set of two or more universal oligo pairs where each oligo in the set has low complimentarily to every other universal oligo in the set, with the exception of its complement. Use of universal oligo chips for detecting cancer markers has many advantages including, but not limited to the following. For example, the universal oligo chips can be used with virtually any downstream application (i.e., the front end assay can detect antibodies, antigens, chemical or biological toxins, metabolites, etc.), yet the chips have standardized hybridization conditions independent of the cancer marker. However, the universal oligo chips can be flexible as well, as different universal oligo sets may be used for different assays, where a particular universal oligo chip may have chip-associated oligos with melting temperatures and/or lengths of X and another universal oligo chip may have chip-associated oligos with melting temperatures and/or lengths of Y. In addition, the universal oligos of the present invention can be engineered to contain sequences for enzyme cleavage for use in some embodiments.

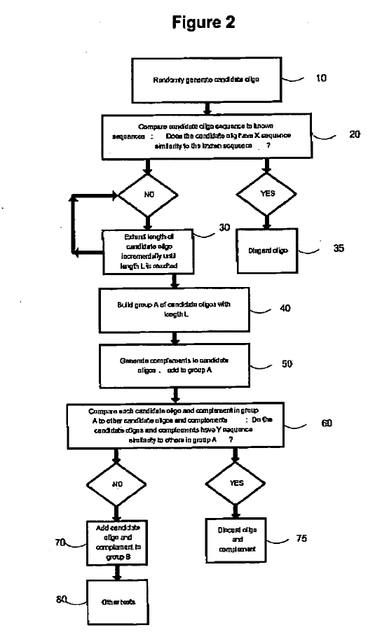

FIG. 2 is a flow chart showing a representative non-limited overview of one embodiment for the creation of universal oligos and a universal oligo set. In step 10, candidate oligo sequences are randomly generated. Typically, such randomly generated sequences will be short, for example, 8-25 nucleotides in length. In one embodiment of the invention, all possible variations of 15-mers (consisting only nucleotides A, T, G and C) are generated and stored in a database. At step 20, each candidate sequence is compared to known sequences, typically, by comparing the candidate sequence to sequences stored in publicly-available and/or custom databases. Custom databases may be databases populated with information from publicly-available databases. Major publicly-available sequence repositories include DDBJ: DNA databank of Japan, EMBL: maintained by EMBL, and GenBank: maintained by NCBI; organelle databases include OGMP: the organelle genome megasequencing program, GOBASE: an organelle genome database, and MitoMap: a human mitochondrial genome database; RNA databases include Rfam: an RNA family database, RNA base: a database of RNA structures, tRNA database: a database of tRNAs, tRNA: tRNA sequences and genes, and sRNA: a small RNA database; comparative and phylogenetic databases include COG: phylogenetic classification of proteins, DHMHD: a human-mouse homology database, HomoloGene: a database of gene homologies across species, Homophila: a human disease to Drosophila gene database, HOVERGEN: a database of homologous vertebrate genes, TreeBase: a database of phylogenetic knowledge, XREF: a database that cross-references human sequences with model organisms; SNP, mutation and variation databases include ALPSbase: a database of mutations causing human ALPS, dbSNP: the single nucleotide polymorphism database at NCBI, and HGVbase: a human genome variation database; alternative splicing databases include ASDB: a database of alternatively spliced genes, ASAP: an alternate splicing analysis tool, ASG: an alternate splicing gallery, HASDB: a human alternative splicing database, AsMamDB: a database of alternatively spliced genes in human, mouse and rat, and ASD: an alternative splicing database at CSHL; and scores of specialized databases include ACUTS: a database of ancient conserved untranslated sequences, AGSD: an animal genome database, AmiGO: a gene ontology database, ARGH: an acronym database, BACPAC: BAC and PAC a database of genomic DNA library info, CHLC: a database of genetic markers on chromosomes, COGENT: a complete genome tracking database, COMPEL: a database of composite regulatory elements in eukaryotes, CUTG: a codon usage database, dbEST: a database of expressed sequences or mRNA, dbGSS: genome survey sequence database, dbSTS: a database of sequence tagged sites (STS), DBTSS: a database of transcriptional start sites, DOGS: a database of genome sizes, EID: the exon-intron database, Exon-Intron: an exon-intron database, EPD: a eukaryotic promoter database, FlyTrap: a HTML-based gene expression database, GDB: the genome database, GeneKnockouts: a database of gene knockout information, GENOTK: a human cDNA database, GEO: a gene expression omnibus NCBI, GOLD: a database of information on genome projects around the world, GSDB: the Genome Sequence DataBase, HGI: TIGR human gene index, HTGS: a database of genomic sequences at NCBI, IMAGE: a database of the largest collection of DNA sequences clones, IMGT: a database of the international ImMunoGeneTics information system, LocusLink: single query interface to sequence and genetic loci, TelDB: ae telomere database, MitoDat: a database of mitochondrial nuclear genes, Mouse EST: a database with information from the NIA mouse cDNA project, MPSS: searchable databases of several species, NDB: a nucleic acid database, NEDO: a human cDNA sequence database, NPD: a nuclear protein database, PLACE: a database of plant cis-acting regulatory DNA elements, RDP: a ribosomal database, RDB: a receptor database at NIHS, Japan, Refseq: the NCBI reference sequence project, RHdb: a database of radiation hybrid physical map of chromosomes, SpliceDB: a database of canonical and non-canonical splice site sequences, STACK: a database of consensus human EST database, TAED: the adaptive evolution database, TIGR: curated databases of microbes, plants and humans, TRANSFAC: the transcription factor database, TRRD: a transcription regulatory region database, UniGene: a database of cluster of sequences for unique genes at NCBI, and UniSTS: a database of non-redundant STS.

For sequence comparison, known sequences act as reference sequences to which the candidate sequences are compared. When using a sequence comparison algorithm, known and candidate sequences are input into a computer, subsequence coordinates are designated if appropriate, and sequence algorithm program parameters are designated. The sequence comparison algorithm then calculates the percent sequence identity or regions of sequence identity for the candidate sequence relative to the known reference sequence, based on the designated program parameters.

In the present invention, universal oligos are designed for detection of cancer markers for the detection and/or diagnosis of cancer. As such, candidate sequences are screened against sequences from mammals, viruses and bacteria contained in a custom database containing information from publicly-available databases.

The determination of percent identity between two or more sequences can be accomplished using a mathematical algorithm. Preferred, non-limiting examples of such mathematical algorithms include, inter alia, the algorithm of Myers and Miller (1988); the search-for-similarity-method of Pearson and Lipman (1988); and that of Karlin and Altschul (1993). Preferably, computer implementations of these mathematical algorithms are utilized. Such implementations include, but are not limited to: CLUSTAL in the PC/Gene program (available from Intelligenetics, Mountain View, Calif.); the ALIGN program (Version 2.0), GAP, BESTFIT, BLAST, FASTA, Megalign (using Jotun Hein, Martinez, Needleman-Wunsch algorithms), DNAStar Lasergene (see www.dnastar.com) and TFASTA in the Wisconsin Genetics Software Package, Version 8 (available from Genetics Computer Group (GCG), 575 Science Drive, Madison, Wis., USA). Alignments using these programs can be performed using the default parameters or parameters selected by the operator. The CLUSTAL program is well described by Higgins. The ALIGN program is based on the algorithm of Myers and Miller; and the BLAST programs are based on the algorithm of Karlin and Altschul. Software for performing BLAST analyses is publicly available through the National Center for Biotechnology Information (http://www.ncbi.nlm.nih.gov/).

If a candidate sequence is found to have sequence similarity above a given limit (however this limit is defined, e.g., X % homology overall or a percentage over a Y basepair stretch of a sequence) during the screening against known sequences, the candidate sequence will be discarded (step 35). If a candidate sequence is found to have sequence homology below a given limit during the screening against known sequences, the candidate sequence will be extended by one or more nucleotides (step 30) and will go through the screening process again.

In a preferred embodiment, the candidate sequence will be extended by one nucleotide at a time (step 30), but will be extended by each of A, T, G and C. For example, if candidate sequence XXXXXXXXXXXXXXX is determined to have sequence homology below the given limit, candidate sequence XXXXXXXXXXXXXXX will then be extended by one nucleotide four times, that is, candidate sequence XXXXXXXXXXXXXXX will be extended to candidate sequence XXXXXXXXXXXXXXXXA, candidate sequence XXXXXXXXXXXXXXXXT, candidate sequence XXXXXXXXXXXXXXXG and candidate sequence XXXXXXXXXXXXXXXC and each of these candidate sequences will be screened as described previously (step 20). The process can be continued until a desired length L is achieved. Once a candidate sequence of desired length L is found, it is placed in a group A of candidate sequences (step 40), and these candidate sequences are used to build a universal oligo set. Though the sequences above are written in conventional 5′-3′ mode, extension can take place from either end.

In building a universal oligo set, sequences complementary to the candidate sequences can be generated and added to the candidate sequences in group A (step 50). At step 60, each candidate sequence and complement in group A are compared to each other candidate sequence and each other complement to determine the extent of sequence similarity (however “sequence similarity” is defined). If a candidate or complement sequence is found to have sequence similarity above a given limit (again, however “sequence similarity” is defined) during the screening at step 60, the candidate sequence and its complement will be discarded (step 75). If it is determined that a candidate sequence and its complement are found to have sequence homology below a given limit during the screening at step 60, the candidate sequence and complement will be added to a group B (step 70). The candidate and complementary sequences in group B may then be subjected to further screening (step 80), using various parameters such as melting temperature (Tm), existence of duplexes, specificity of hybridization, existence of a GC clamp, existence of hairpins, existence of sequence repeats, dissociation minimum for a 3′ dimer, dissociation minimum for the 3′ terminal stability range, frequency threshold, or maximum length of acceptable dimers and the like.

The universal oligos can be 1 to 10000 bases in length, preferably 10 to 1000 bases in length, more preferably 10-500 bases in length and more preferably about 25 to about 100 bases in length. Additionally, the universal oligos may be DNA, RNA or PNA (peptide nucleic acid) and can include non-naturally occurring subunits, sequences and/or moieties. PNA includes peptide nucleic acid analogs. The backbones of PNA are substantially non-ionic under neutral conditions, in contrast to the highly charged phosphodiester backbone of naturally occurring nucleic acids. This results in, inter alia, two advantages. First, the PNA backbone exhibits improved hybridization kinetics. PNAs have larger changes in the melting temperature (Tm) for mismatched versus perfectly matched basepairs. DNA and RNA typically exhibit a 2-4° C. drop in Tm for an internal mismatch. With the non-ionic PNA backbone, the drop is closer to 7-9° C. This allows for better detection of mismatches. Similarly, due to their non-ionic nature, hybridization of the bases attached to these backbones is relatively insensitive to salt concentration. This can be advantageous in certain embodiments, as a reduced salt hybridization solution has a lower Faradaic current than a physiological salt solution (in the range of 150 mM).

Conjugation of the capture-associated universal oligos to the capture moieties may be performed in numerous ways, providing it results in a capture moiety possessing both epitope-specific binding to capture the cancer marker as well as providing it does not restrict nucleic acid hybridization functionalities in embodiments where a cleavage is not performed, to allow detection of the bound cancer marker. For example, nucleic acid-antibody conjugates can be synthesized by using heterobifunctional cross-linker chemistries to covalently attach single-stranded DNA labels through amine or sulfhydryl groups on an antibody to create a capture agent of the invention (see, e.g., Hendricksen E R, Nucleic Acids Res., 23(3):522-9(1995)). In another example, covalent single-stranded DNA-streptavidin conjugates, capable of hybridizing to complementary surface-bound oligonucleotides, are utilized for the effective immobilization of biotinylated antibodies. See, e.g., Niemeyer C M, et al., Nucleic Acids Res.; 31(16):90 (1995). Many other nucleic acid molecular conjugates are described, for example, in Heidel J et al., Adv Biochem Eng Biotechnol.; 99:7-39 (2005). Additional methods of creating antibody-oligo conjugates, both those existing and under development, will be apparent to one skilled in the art upon reading the present disclosure, and such methods are intended to be captured within the methods of the invention.

In accordance with one embodiment of the present invention, one oligo of a universal oligo pair, the chip-associated universal oligo, is immobilized (directly or indirectly) onto an electrochemical surface. Although a metal electrode (e.g., gold, aluminum, platinum, palladium, rhodium, ruthenium, any metal or other material having a free electron in its outer most orbital) is preferably employed as the surface for immobilizing the chip-associated universal oligo, other surfaces such as photodiodes, thermistors, ISFETs, MOSFETs, piezo elements, surface acoustic wave elements, and quartz oscillators may also be employed. By “electrode” herein is meant a composition, which, when connected to an electronic device, is able to conduct, transmit, receive or otherwise sense, detect or facilitate the detection of a current or charge. Such current or charge is preferably capable of being converted into a detectable signal. Alternatively an electrode can be defined as a composition, which can apply a potential to and/or pass electrons to or from a chemical moiety. Preferred electrodes are known in the art and include, but are not limited to, certain metals and their oxides, including gold; platinum; palladium; silicon; aluminum; titanium, metal oxide electrodes including platinum oxide, titanium oxide, tin oxide, indium tin oxide, palladium oxide, silicon oxide, aluminum oxide, molybdenum oxide (Mo₂, O₆), tungsten oxide (WO₃) and ruthenium oxides; carbon (including glassy carbon electrodes, graphite, pyrolytic graphite, carbon fiber, and carbon paste); and semiconductor electrodes, such as Si, Ge, ZnO, CdS, TiO₂ and GaAs. Preferred electrodes include gold, silicon, platinum, carbon and metal oxide electrodes, with gold being particularly preferred. The electrode may also be covered with conductive compounds to enhance the stability of the electrodes immobilized with probes or nonconductive (e.g., insulative materials). Monomolecular films or biocompatible materials may also be employed to coat or partially coat the electrodes.

The electrodes described herein are presumed to be a flat surface, which is only one of the possible conformations of the electrode. The conformation of the electrode depends upon the detection method employed. For example, flat planar electrodes may be preferred for electrochemical detection methods, thus requiring addressable locations for synthesis and/or detection. In a preferred embodiment, the detection electrodes are formed on a glass or polymer substrate. The discussion herein is generally directed to the formation of gold electrodes, but as will be appreciated by those in the art, other electrodes can be used as well. The substrate can comprise a wide variety of materials, as will be appreciated by those in the art, with glass, polymers and printed circuit board (PCB) materials being particularly preferred. Thus, in general, the suitable substrates include, but are not limited to, fiberglass, Teflon, ceramics, glass, silicon, mica, plastic (including acrylics, polystyrene and copolymers of styrene and other materials, mylar, polypropylene, polyethylene, polybutylene, polycarbonate, polyurethanes, Teflon™, and derivatives thereof, etc.), GETEK (a blend of polypropylene oxide and fiberglass), and other materials typically employed and readily known to those of ordinary skill in the art.

As is generally known in the art, one or a plurality of layers may be used, to make either “two-dimensional” (e.g. all electrodes and interconnections in a plane) or “three dimensional” substrates. Three-dimensional systems frequently rely on the use of drilling or etching, followed by electroplating with a metal such as copper, such that the “through board” interconnections are made, or comprise porous structures similar to, inter alia, xeolites in structure.

Accordingly, in a preferred embodiment, the present invention provides universal oligo chips that comprise substrates comprising a plurality of electrodes, preferably gold, platinum, palladium or semiconductor electrodes. In addition, each electrode is capable of interconnection that is attached to the electrode at one end and is ultimately attached to a device that can control the electrode and/or receive the signal transmitted via conductive means in contact with the electrode. That is, each electrode is independently addressable. The substrates can be part of a larger device comprising a detection chamber that exposes a given volume of sample to the detection electrode. Generally, the detection chamber ranges from about 1 pl to 1 ml, with about 10 μl to 500 μl being preferred. As will be appreciated by those in the art, depending on the experimental conditions and assay, smaller or larger volumes may be used. The volumes and concentrations employed are typically empirically determines using methods readily known to those of ordinary skill in the art.

In certain embodiments, the detection chamber and electrode are part of a cartridge that can be placed into a device comprising electronic components selected from the group comprising potentiometers, AC/DC voltage source, ammeters, processors, displays, temperature controllers, light sources, and the like. In a typical embodiment, the interconnections from each electrode are positioned such that upon insertion of the cartridge into the device, connections between the electrodes and the electronic components are established. The device can also comprise a means for controlling the temperature, such as a peltier block, that facilitates the conditions employed in the hybridization reaction.

In certain preferred embodiments, the electrode is first coated with a biocompatible substance (such as dextran, carboxylmethyldextran, other hydrogels, polypeptides, polynucleotides, biocompatible and/or bio-inert matrices or the like). The chip-associated universal oligo is immobilized to such biocompatible substance.

The chip-associated universal oligos may be immobilized onto the electrodes directly or indirectly by covalent bonding, ionic bonding and physical adsorption. Examples of immobilization by covalent bonding include a method in which the surface of the electrode is activated and the nucleic acid molecule is then immobilized directly to the electrode or indirectly through a cross linking agent. Yet another method using covalent bonding to immobilize a chip-associated universal oligo includes introducing an active functional group into an oligo followed by direct or indirect immobilization. The activation of the surface may be conducted by electrolytic oxidation in the presence of an oxidizing agent, or by air oxidation or reagent oxidation, as well as by covering with a film. Useful cross-linking agents include, but are not limited to, silane couplers such as cyanogen bromide and gamma-aminopropyl triethoxy silane, carbodiimide and thionyl chloride and the like. Useful functional groups to be introduced to the oligo may be, but are not limited to, sulfide, disulfide, amino, amide, amido, a carboxyl, a hydroxyl, carbonyl, oxide, phosphate, sulfate, aldehyde, keto, ester and mercapto groups. Other highly reactive functional groups may also be employed using methods readily known to those of ordinary skill in the art.

To facilitate the screening and/or detection of multiple cancer markers in a sample, multiple electrodes, or an electrode with multiple chip-associated universal oligos attached, preferably in a predetermined configuration are employed. In such a configuration, a plurality of electrodes each having a distinct chip-associated universal oligo affixed thereto or otherwise associated therewith are arranged in predetermined configuration. In a preferred embodiment, the voltage applied to each electrode is equal. Additionally, to verify the hybridization of a particular chip-associated universal oligo, the electrochemical detection device preferably includes a switch circuit, a decoder circuit, and/or, a timing circuit to apply the voltage to the individual electrodes and to receive the output signal from each of the electrodes. In certain preferred embodiments, the signals are distinguished via the character of the signal, irrespective of configuration.

Alternatively, the capture-associated universal oligos and chip-associated universal oligos can be used in traditional optical detection methods well known in the art. In this case, the chip-associated universal oligos may be synthesized in situ (see, e.g., U.S. Pat. Nos. 5,744,305; 5,753,788; 5,770,456; 5,889,165; 6,346,413; 6,506,558; 6,566,495; and 6,600,031) or by physical spotting of the chip-associated universal oligos with the aid of robotic arraying equipment or through electronic addressing on a solid substrate such as glass. The matrix or material that serves as a substrate “chip” on which chip-associated universal oligos are arrayed may be of any type of solid support, and the association may be covalent or noncovalent. The solid support can take on a variety of shapes and compositions, including microparticles, beads, porous and impermeable strips and membranes, the interior surface of reaction vessels such as test tubes and microtiter plates, and the like. Methods for attaching a desired reaction partner to a selected solid support will be a matter of routine skill to the worker in the field.

For example, covalent immobilization of nucleic acids on a support may be used, and a wide variety of support materials and coupling techniques can be employed. For example, the nucleic acids can be coupled to phosphocellulose through phosphate groups activated by carbodiimide or carbonyldiimidazole (see, e.g., Bautz, E. K. F., and Hall, B. D., Proc. Nat'l. Acad. Sci. USA 48:400-408 (1962); and Shih, T. Y., and Martin, M. A., Biochem. 13:3411-3418 (1974)). Also, diazo groups on m-diazobenzoyloxymethyl cellulose can react with guanine and thymidine residues of the polynucleotide (see, e.g., Noyes, B. E., and Stark, G. R., 5:301-310; and Reiser, J., et al, Biochem. Biophys. Res. Commun. 85:1104-1112 (1978)). Polysaccharide supports can also be used with coupling through phosphodiester links formed between the terminal phosphate of the polynucleotide and the support hydroxyls by water soluble carbodiimide activation (see, e.g., Richwood, D., Biochim. Biophys. Acta 269:47-50 (1972); and Gilham, P. T., Biochem. 7:2809-2813 (1968)), or by coupling nucleophilic sites on the polynucleotide with a cyanogen bromide activated support (see, e.g., Arndt-Jovin, D. J., et al, Eur. J. Biochem. 54:411-418 (1975); and Linberg, U., and Eriksson, S., Eur. J. Biochem. 18:474-479 (1971)). Further, the 3′-hydroxyl terminus of the nucleic acid can be oxidized by periodate and coupled by Schiff base formation with supports bearing amine or hydrazide groups (see, e.g., Gilham, P. T., Method. Enzymol. 21:191-197 (1971); and Hansske, H. D., et al, Method. Enzymol. 59:172-181 (1979)). Supports having nucleophilic sites can be reacted with cyanuric chloride and then with the polynucleotide (see, e.g., Hunger, H. D., et al, Biochim. Biophys. Acta 653:344-349 (1981)).

In general, any method can be employed for immobilizing the nucleic acid, provided that the chip-associated oligo sequence is available for hybridization to the capture-associated oligo or polymerization products. Particular methods or materials are not critical to the present invention.

A particularly attractive alternative to employing directly immobilized nucleic acid is to use an immobilizable form of nucleic acid which allows hybridization to proceed in solution where the kinetics are more rapid. Normally in such embodiment, one would use a chip-associated oligo which comprises a reactive site capable of forming a stable covalent or noncovalent bond with a reaction partner and obtain immobilization by exposure to an immobilized form of such reaction partner. Preferably, such reactive site in the chip-associated oligo is a binding site such as a biotin or hapten moiety which is capable of specific noncovalent binding with a binding substance such as avidin or an antibody which serves as the reaction partner.

Essentially any pair of substances can comprise the reactive site/reactive partner pair which exhibit an appropriate affinity for interacting to form a stable bond, that is a linking or coupling between the two which remains substantially intact during the subsequent assay steps, principally the separation and detection steps. The bond formed may be a covalent bond or a noncovalent interaction, the latter being preferred especially when characterized by a degree of selectivity or specificity. In the case of such preferred bond formation, the reactive site on the chip-associated oligo will be referred to as a binding site and the reaction partner as a binding substance with which it forms a noncovalent, commonly specific, bond or linkage.

In such preferred embodiment, the binding site can be present in a single stranded hybridizable portion or in a single or double stranded nonhybridizable portion of the chip-associated oligo or can be present as a result of a chemical modification of the chip-associated oligo. Examples of binding sites existing in the nucleotide sequence are where the nucleic acid comprises a promoter sequence (e.g., lac-promoter, trp-promoter) which is bindable by a promoter protein (e.g., bacteriophage promoters, RNA polymerase), or comprises an operator sequence (e.g., lac operator) which is bindable by a repressor protein (e.g., lac repressor), or comprises rare, antigenic nucleotides or sequences (e.g., 5-bromo or 5-iododeoxyuridine, Z-DNA) which are bindable by specific antibodies (see also British Pat. No. 2,125,964). Binding sites introduced by chemical modification of the polynucleotide comprised in the chip-associated oligo are particularly useful and normally involve linking one member of a specific binding pair to the chip-associated oligo. Useful binding pairs from which to choose include biotin/avidin (including, for example, egg white avidin and streptavidin), haptens and antigens/antibodies, carbohydrates/lectins, enzymes/inhibitors, and the like. Where the binding pair consists of a proteinaceous member and a nonproteinaceous member, it will normally be preferred to link the nonproteinaceous member to the chip-associated oligo since the proteinaceous member may be unstable under the denaturing conditions of hybridization of the chip-associated oligo to the capture-associated oligo or to the polymerization products. Preferable systems involve linking the chip-associated oligo with biotin or a hapten and employing immobilized avidin or anti-hapten antibody reagent, respectively.

Labels are attached to the capture-associated universal oligos (or the capture moiety) and detected with an array reader that quantitates the level of optical activity (typically, fluorescence) and identifies the location of the hybridization event. Typically the reader involves confocal optical detection as discussed in detail infra. In the present embodiment, the label is added directly to the capture-associated universal oligo or to capture moiety (e.g., the antibody). Means of attaching labels to nucleic acids are well known to those of skill in the art and include, e.g., end-labeling by kinasing the universal oligo and subsequent attachment (e.g., ligation) of a nucleic acid linker joining the sample nucleic acid to a label (e.g., a fluorophore). Useful labels this embodiment of the present invention include fluorescent dyes (e.g., fluorescein, texas red, rhodamine, green fluorescent protein, and the like. Patents teaching the use of labels include, inter alia, U.S. Pat. Nos. 3,817,837; 3,850,752; 3,939,350; 3,996,345; 4,277,437; 4,275,149; and 4,366,241.

If amplification is performed as described in detail infra, the capture-associated universal oligo and the chip-associated universal oligo will have the same or substantially the same sequence and the amplification products will be complementary (or substantially complementary) to the capture-associated universal oligos and the chip-associated universal oligos.

Assay and Detection

The universal oligos and universal oligo chips are used in a system comprising capture-associated universal oligos, where the capture moiety is one or more moieties specific for cancer markers such as those listed in Table 1. The capture-associated universal oligos are contacted/mixed with a sample that is suspected of containing the cancer markers, under conditions that if a cancer marker is present, the capture moiety can react with, i.e., bind or other associate with/to the cancer marker. In most embodiments, the capture-associated universal oligos conjugated to the capture moieties are added in excess relative to the amount of cancer marker suspected to be present in the sample.

In one embodiment, the capture moiety is one or more antibodies specific for cancer markers that are antigens. The immobilized binding partners can be a naturally-occurring or synthetic epitope of the antigens. If multiple capture-associated universal oligos are used, each having an antibody specific for a different antigen or different epitope of the same antigen, multiple immobilized binding partners are used to facilitate the removal/separation of unreacted capture-associated universal oligos (those with antibodies that did not react with target antigen in the sample). In such a detection method, multiple different target antigens may be screened/detected simultaneously. The advantage of a simultaneous accurate detection method includes an increased speed at which multiple suspected antigens can be eliminated.

With these concepts in mind, in one application of one embodiment of the invention, capture-associated universal oligos are conjugated to antibodies and the cancer marker of interest is an antigen. In accordance with this embodiment the invention the following elements are included: (1) a chip-associated universal oligo immobilized on a surface, (2) a capture-associated universal oligo that is complementary to the chip-associated universal oligo, where the capture-associated universal oligo is conjugated to an antibody corresponding to one or more cancer markers, (3) immobilized binding partners, and (4) a sample suspected of containing the one or more cancer markers. In one aspect, the capture-associated universal oligo is contacted with the sample to form a first mixture, then the first mixture is contacted with the immobilized binding partners. The unreacted capture-associated universal oligos are captured by the immobilized binding partners, thereby removing the unreacted capture-associated universal oligos from solution. The solution phase of the mixture is then contacted with the chip-associated universal oligos, followed by detection as otherwise described herein. Alternatively, the reacted oligo-antibody-antigen moieties can be immobilized with an immobilized binding partner to the antibody/antigen complex, leaving the unreacted oligo-antibody molecules in solution. Other variations on this preferred embodiment include one or more other aspects of the invention described herein or such other modification known to those of ordinary skill in the art.

This embodiment most frequently is employed in a multi-target (so-called multiplexed) format, allowing for the screening of multiple cancer markers simultaneously. Such embodiments include providing (1) a detection device comprising chip-associated universal oligos, (2) a set of capture-associated universal oligos, (3) a sample suspected of containing cancer marker antigens or epitopes, and (4) immobilized binding partners to the antibodies of the capture-associated universal oligos. The method comprises mixing/contacting the sample with the capture-associated universal oligos under reaction conditions that allow the antibodies to capture cancer markers present in the sample to form a first mixture. The first mixture is then mixed/contacted with the immobilized binding partners to the antibodies where the antibodies that have not reacted with cancer markers in the sample react with the immobilized binding partners to form an immobilized phase and a solution phase. The solution phase comprises the capture-associated universal oligos that have reacted with cancer markers in the sample and the immobilized phase comprises the capture-associated universal oligos that did not bind cancer markers and instead bound the immobilized binding partners. The solution is introduced to the universal oligo chip and the detection device under conditions such that the capture-associated universal oligos present will hybridize to a complementary chip-associated universal oligo, generating a signal.

Alternatively, the reacted capture-associated universal oligo complex can be captured (e.g., by an antibody that recognizes a different epitope of the cancer marker or the capture moiety/cancer marker complex) leaving the unreacted capture-associated universal oligos in solution. The immobilized phase is separated, and the reacted capture-associated universal oligo complex is then released into solution and introduced to the universal oligo chip and to the detection device under reaction conditions such that the capture-associated universal oligos and chip-associated universal oligos may hybridize to each other. A signal generated by the hybridization of complementary capture-associated universal oligos and chip-associated universal oligos.

The binding reaction between the cancer markers in the sample and the capture-associated universal oligos is performed in solution, in a physiological buffer such as phosphate buffered saline (PBS) supplemented with a non-specific blocking agent, such as fetal or new-born calf serum, and may be used when the cancer marker to be detected is normally found under physiological conditions. However, the methods of the present invention are not limited to detecting cancer markers only found in physiological conditions. Those of skill in the art would appreciate and understand that different ligands may be used in different conditions without affecting the ability to bind the particular cancer marker to be detected. The binding reaction can be performed at a temperature within the range of 0° C. to 100° C., preferably at a temperature between 2° C. and 40° C., and more preferably within the range of about 4° C. to about 37° C., and most preferably within the range of about 18° C. to about 25° C. The binding reaction is typically conducted from about 5 minutes to 12 hours, preferably from about 10 minutes to 6 hours, and more preferably from about 15 minutes to 1 hour. The duration of the binding reaction depends on several factors, including the temperature, suspected concentration of the cancer marker, ionic strength of the sample, and the like. For example, a binding reaction may require 15 minutes incubation at a temperature of 18° C., or 30 minutes incubation at a temperature of 4° C.

Since often cancer marker antigens and antibodies are involved in the capture reaction as in the binding reaction, typically the capture reaction between the reacted and unreacted capture-associated universal oligos and the immobilized binding partners is performed under conditions much like the binding reaction. The capture reaction also takes place in solution, in a physiological buffer such as phosphate buffered saline (PBS) supplemented with a non-specific blocking agent, such as fetal or new-born calf serum. The capture reaction can be performed at a temperature within the range of 0° C. to 100° C., preferably at a temperature between 2° C. and 40° C., and more preferably within the range of about 4° C. to about 37° C., and most preferably within the range of about 18° C. to about 25° C. The capture reaction is typically conducted from about 5 minutes to 12 hours, preferably from about 10 minutes to 6 hours, and more preferably from about 15 minutes to 1 hour. Those of skill in the art would appreciate and understand the particular the specific time required for the reaction to be performed.

The removal of excess, unreacted capture-associated universal oligos can be achieved by providing immobilized binding partner(s) to the specific capture moiety that is conjugated to the capture-associated universal oligos. The immobilized binding partner is bound to a matrix that is a vessel wall or floor. Alternatively, the matrix may be a column or filter, such as Sepharose 2B, Sepharose 4B, Sepharose 6B, CNBR-Activated Sepharose 4B, AH-Sepharose 4B, CH-Sepharose 4B, Activated CH-Sepharose 4B, Epoxy-Activated Sepharose 6B, Activated Thiol-Sepharose 4B, Sephadex, CM-Sephadex, ECH-Sepharose 4B, EAH-Sepharose 4B, NHS-Activated Sepharose or Thioporpyl Sepharose 6B, etc., all of which are supplied by Pharmacia; Bio-Gel A, Cellex, Cellex AE, Cellex-CM, Cellex PAB, BIO-GEL P, Hydrazide BIO-GEL P, Aminoethyl Bio-Gel P, Bio-Gel CM, Affi-Gel 10, Affi-Gel 15, Affi-Prep 10, Affi-Gel HZ, Affi-Prep HZ, Affi-Gel 102, CM Bio-Gel A, Affi-Gel Heparin, Affi-Gel 501 or Affi-Gel 601, etc., all of which are supplied by Bio-Rad; Chromagel A, Chromagel P, Enzafix P-HZ, Enzafix P-SH OR Enzafix P-AB, etc., all of which are supplied by Wako Pure Chemical Industries Ltd.; AE-Cellurose, CM-Cellurose or PAB Cellurose etc., all of which are supplied by Serva, over which the mixture of reacted and unreacted conjugated nucleic acid molecules can be passed. Similarly, the matrix may include a suspension of particulate matter in a solution, such as microscopic and/or macroscopic beads/particles, where the immobilized binding partner is immobilized on the beads or particle such as polystyrene-, cellulose-, latex-, silica-, polyaminostyrene-, agarose-, polydimethylsiloxane-, or polyvinyl-based beads. In a method using particles, the unreacted nucleic acid molecules will be retained on the semi-solid support created by the particles, whereas the reacted nucleic acid molecules will be eluted through the semi-solid support. Thus, only those capture-associated universal oligos that have bound the particular cancer marker will be available for hybridization. Alternatively, the particles can include an immobilized binding partner specific for the cancer marker or for the cancer marker/capture moiety complex. In this embodiment, only those capture-associated universal oligos conjugated to a capture moiety that has reacted with the cancer marker in the sample will be retained on the particles or matrix, and the unreacted nucleic acid molecules will pass through. The retained, reacted capture-associated universal oligos then may be selectively released/eluted by known methods including but not limited to the cleavage step, discussed in detail below.

When employing suspensions of particulate matter in a solution, unreacted nucleic acid molecules can be separated from the reacted nucleic acid molecules by techniques such as centrifugation, size exclusion chromatography, filtration and the like. In a method using beads, in particular magnetic beads, the separation step can be achieved by applying a magnetic field to the magnetic beads. In some embodiments, the beads will bind with the unreacted capture moieties, leaving the reacted capture moieties in solution and available for hybridization. In other embodiments, the beads will bind with the reacted capture moieties, leaving the unreacted capture moieties in solution. In addition, either the suspension or bead techniques can employ a particle or bead having a secondary capture moiety specific for the cancer marker to be detected. In this instance only those capture-associated universal oligos are that have reacted with cancer markers in the sample will be retained on the beads, and the unreacted capture-associated universal oligos are separated from the suspension by known techniques including, but not limited to, centrifugation, size exclusion chromatography, filtration, magnetism and the like. As discussed above, in this particular embodiment of the invention, the retained, reacted capture-associated universal oligos can be selectively released/eluted by known methods including, but not limited to, the cleavage step, as discussed.

The capture-associated universal oligos preferably are provided to the capture reaction in excess, with the excess (i.e., unreacted) capture-associated universal oligos being removed prior to hybridization. This excess is typically determined relative to the suspected level of cancer markers present in the sample. This relative excess can be from about 1:1 to 1000000:1, preferably 2:1 to about 10000:1, and more preferably from about 4:1 to 1000:1, and most preferably from 5:1 to 100:1. For example, when the capture moiety is an antibody, typically, an excess of capture moiety can be created by adding 1 μg of the capture-associated universal oligo to a sample suspected of containing up to 1 million cancer markers to be detected. This ratio gives rise to a molar ratio of typically about 4:1, but can vary dependant upon the molecular mass of the antibody and the cancer marker to be detected.

In an alternative embodiment, the immobilization binding partners bind to the capture moieties that have bound the cancer markers instead of binding the unreacted capture moieties in an “antibody capture” scenario. For example, in the case where the capture moiety is an antibody, the oligo-conjugated antibody is mixed with a sample suspected of containing the cancer marker, in this case, an antigen. In contrast to other embodiments described thus far, the resulting first mixture is then contacted with an immobilized antibody to the same cancer marker, immobilizing the [oligo-antibody-cancer marker] complex by formation of an [oligo-antibody-cancer marker-antibody] complex in a second mixture. The second mixture will, typically, have a solution phase comprising the oligo-conjugated capture moiety (in this example, an antibody) that did not capture a cancer marker (in this case, an antigen) and an immobilized phase comprising the [oligo-antibody-cancer marker-antibody] complex. The solution phase can be removed from the reaction mixture by known methods including, but not limited to decanting, centrifugation, washing, etc. The immobilized [oligo-antibody-cancer marker-antibody] complex is then released (or, in some embodiments, the capture-associated oligo is cleaved from the [oligo-antibody-cancer marker-antibody] complex) into solution, where the solution is transferred to, e.g., an electrochemical detection device for detection. Through this process, the only capture-associated oligos present in the third mixture transferred and introduced into the electrochemical device are those capture-associated oligos that correspond to the cancer marker (in this case, an antigen) present in the sample. The immobilized binding partner antibody can be the same antibody conjugated to the capture-associated oligo, can recognize a different epitope of the cancer marker antigen, or can recognize the capture moiety/cancer marker complex. As in other embodiments, multiple different capture-associated oligo/capture moiety conjugates can be employed (so-called multiplexing), thereby allowing for the simultaneous screening and detection of multiple cancer markers from a single sample.

In other preferred embodiments, the antigen and antibody are reversed and, accordingly, the immobilization and subsequent steps or reaction states are reversed or otherwise alternatively configured

In some embodiments of the invention, cleavage of the antibody from the capture-associated universal oligos following separation of reacted and unreacted molecules, but prior to hybridization, is preferable. This situation may arise when the reacted capture-associated universal oligos have been selectively bound to a capture moiety that may interfere with hybridization, or detection, because of the physical size or the presence of local areas of electron density on the surface of the capture moiety. Cleavage can be achieved by, for example, a digestive enzyme, i.e., an enzyme that causes hydrolysis of a bond in a molecule, (e.g., proteolytic enzymes, lipases, phosphatases, phosphodiesterases, esterases, etc.), endonucleases, exonucleases, a restriction endonuclease (e.g., EcoRI), or a flap endonuclease (e.g., FEN-1, RAD2, XPG, etc.). The choice of cleavage method will depend on the nature of the conjugation of the capture moiety to the capture-associated universal oligo, and the moiety to be removed via the cleavage reaction. For example, photocleavage may be employed where a photocleavable phosphoramidite is used in lieu of a restriction site. Those of skill in the art will readily appreciate and understand the circumstances under which one particular method of cleavage would be preferred over another method of cleavage.

For example, a digestive enzyme, such as trypsin, can be used when an antibody is conjugated to the capture-associated universal oligo through some peptide linkage; a restriction endonuclease can be used when there is a specific sequence present in the capture-associated universal oligo, susceptible to the particular restriction endonuclease, between the portion of the capture-associated universal oligo that is complementary to the chip-associated universal oligo molecule and the portion of the capture-associated universal oligo that is conjugated to the capture moiety. In preferred embodiments, restriction sites and restriction endonucleases are chosen that allow cleavage of single stranded nucleic acids. Likewise, a flap endonuclease, such as RAD2, or XPG, could be used when there is a specific structure present in the capture-associated universal oligo, susceptible to the particular flap endonuclease, between the portion of the capture-associated universal oligo that is complementary to the chip-associated universal oligo molecule and the portion of the capture-associated universal oligo that is conjugated to the capture moiety. Those of skill in the art would appreciate and understand the particular types of structure susceptible to flap endonuclease cleavage.

Where it is intended that a restriction endonuclease will be used to separate the antibody from the capture-associated universal oligo, the capture-associated universal oligo will be engineered to contain a specific restriction site between the portion of the capture-associated universal oligo that is complementary to the chip-associated universal oligo molecule and the portion of the capture-associated universal oligo that is conjugated to the antibody. This restriction site will be designed, and the appropriate restriction endonuclease selected, to cleave only in the portion of the capture-associated universal oligo that is conjugated to the antibody and not in the region of complementarily to the chip-associated nucleic acid molecule.

In those embodiments where such cleavage is performed, the cleavage reaction is performed after the capture reaction has been completed and after a selective purification reaction is employed in order to segregate the desired reaction product (i.e., the composition comprising the capture moiety and cancer marker); for example, the reaction product can be subjected to a secondary capture (e.g., using a secondary immobilized antibody) followed by separation and wash procedures. The immobilized capture-associated universal oligo complex may then be eluted or otherwise separated from its immobilized substrate and the resulting solution containing the released capture-associated universal oligo transferred to chip for hybridization and detection.

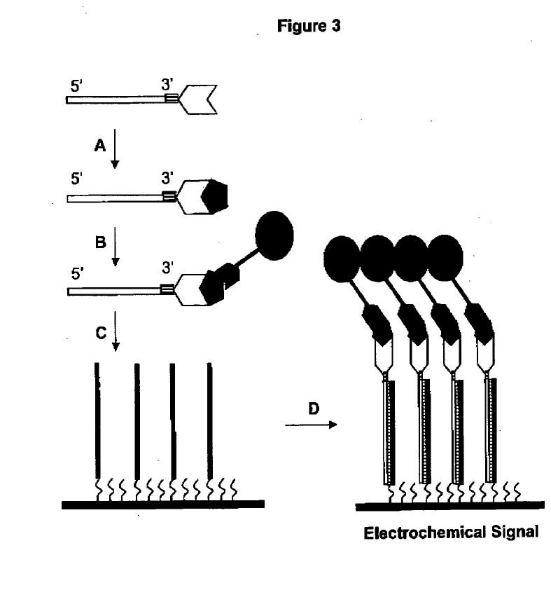

In preferred embodiments, it may be beneficial to use isothermal amplification to increase the number of nucleic acids available for binding to the chip-associated universal oligos, thus enhancing the signal created through complementary binding. As shown in FIG. 3, following binding of the cancer marker to the capture moiety and isolation from the sample, an oligonucleotide encoding the 5′ to 3′ polymerase recognition sequence is introduced to the capture moiety-cancer marker complex, and its binding to the complex creates a double-stranded polymerase recognition site (Step A). Following annealing of the oligonucleotide, an excess of single nucleotides and the appropriate polymerase are added to a solution containing the isolated capture moiety-cancer marker complex, and conditions created to allow for polymerization and linear amplification. Step B comprises (i) exposing the template nucleic acid complex to an aqueous solution comprising the polymerase and an excess of NTP or dNTP and (ii) permitting the polymerase and reactants to create an intermediate duplex comprising a double stranded DNA having a first 5′ end which bears a phage-encoded RNA polymerase recognition site. This reaction continues as the polymerase displaces the double-stranded nucleic acid, resulting in multiple copies of the polymerization products (Step C). In such an embodiment, the chip-associated oligo will have the same sequence as the capture-associated oligo, and both will be complementary to the linear polymerization products.

In a preferred embodiment, the polymerase recognition site created by this double stranded region is a phage-encoded RNA polymerase recognition sequence. Exemplary polymerases useful in such isothermal amplification reactions include RNA phage polymerases, including but not limited to T3 polymerase, SP6 polymerase, and T7 polymerase. In one embodiment, a mutant phage-encoded polymerase (e.g., the T7 RNA polymerase mutant Y639F or S641A) is used to allow creation of DNA rather than RNA. This embodiment increases the stability of the synthesized nucleic acids for binding to the electrode, and obviates the problem of RNAse activity. Such mutant polymerases include T7 DNA polymerase, as disclosed in U.S. Pat. No. 6,531,300, U.S. Pat. No. 6,107,037, U.S. Pat. No. 5,849,546, and Padilla and Sousa, Nucleic Acids Res 27(6):1561-1563 (1999).

A number of different nucleotides can be used in the isothermal linear amplification reaction. These include not only the naturally occurring nucleoside mono-, di-, and triphosphates: deoxyadenosine mono-, di- and triphosphate; deoxyguanosine mono-, di- and triphosphate; deoxythymidine mono-, di- and triphosphate; and deoxycytidine mono-, di- and triphosphate (referred to herein as dA, dG, dT and dC or A, G, T and C, respectively). Nucleotides also include, but are not limited to, modified nucleotides and nucleotide analogs such as deazapurine nucleotides, e.g., 7-deaza-deoxyguanosine (7-deaza-dG) and 7-deaza-deoxyadenosine (7-deaza-dA) mono-, di- and triphosphates, deutero-deoxythymidine (deutero-dT) mon-, di- and triphosphates, methylated nucleotides e.g., 5-methyideoxycytidine triphosphate, 13C/15N labeled nucleotides and deoxyinosine mono-, di- and triphosphate. When using dNTPs and a traditional RNA polymerase, dUTP is substituted for dTTP. For those skilled in the art, it will be clear upon reading the present disclosure that modified nucleotides and nucleotide analogs that utilize a variety of combinations of functionality and attachment positions can be used in the present invention.

Asymmetric amplification using a heat stable polymerase such as Thermus aquaticus polymerase can also be used to create polymerization products complementary to the chip-associated oligos. Suitable methods of asymmetric amplification are described in U.S. Pat. No. 5,066,584, which is incorporated by reference in its entirety. When this technique is used, an oligonucleotide complementary to the 3′ end of the capture-associated oligo is used under conditions to create a series of polymerization products. In such an embodiment, the chip-associated oligo will have the same sequence as the capture-associated oligo, and both will be complementary to the asymmetric polymerization products.

Amplification using the Phi29 polymerase may also be used to create polymerization products complementary to the chip-associated oligo. Such methods are described in U.S. Pat. No. 5,712,124 and U.S. Pat. No. 5,455,166, both of which are incorporated by reference in their entirety. In brief, the Phi29 polymerase method will allow amplification of the capture-associated oligo at a single temperature by utilizing the Phi29 polymerase in conjunction with an endonuclease that will nick the polymerized strand, allowing the polymerase to displace the strand without digestion while generating a newly polymerized strand. As with asymmetric amplification, an oligonucleotide complementary to the 3′ end of the capture-moiety associated nucleic acid is used under conditions to create a series of polymerization products complementary to the capture-associated oligo. In such an embodiment, the chip-associated oligo will have the same sequence as the capture-associated oligo, and both will be complementary to the asymmetric polymerization products.

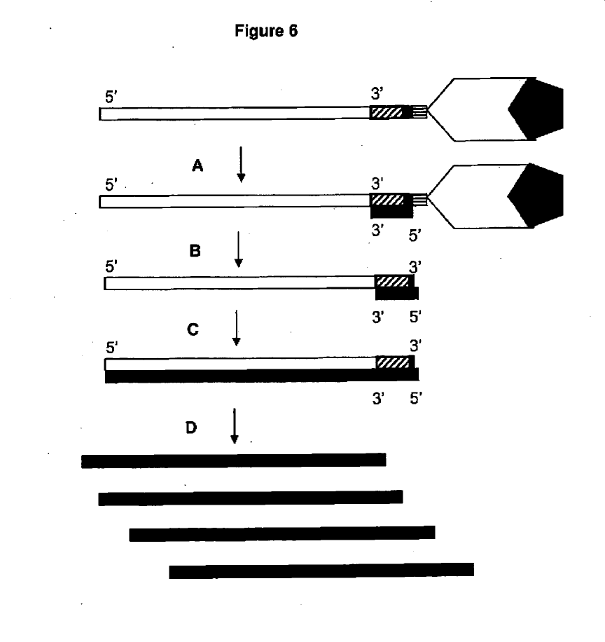

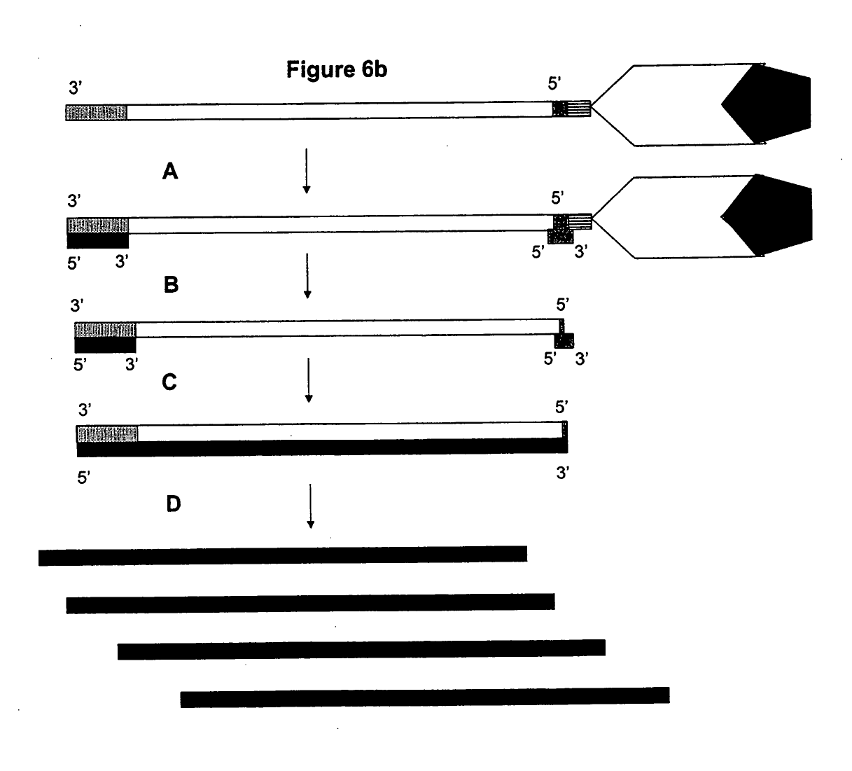

In a particular embodiment of the invention, the capture moiety-cancer marker complex is cleaved from the capture-associated oligo prior to linear or asymmetric amplification. A representative, non-limiting illustration of one such embodiment is illustrated in FIG. 6. Following binding of the cancer marker to the capture moiety and isolation from the sample, an oligonucleotide encoding the 5′ to 3′ polymerase recognition sequence and a restriction endonuclease sequence is introduced to the capture-associated oligo, and its binding to the capture-associated oligo creates both a double-stranded polymerase recognition site and a restriction endonuclease cleavage site (Step A). Following annealing of the oligonucleotide to the capture-associated oligo, the complex is exposed to the appropriate restriction endonuclease under conditions to allow the cleavage of the capture moiety-cancer marker from the capture-associated oligo (Step B). The restriction endonuclease is then optionally inactivated (e.g., through heat inactivation by exposing the solution to a temperature of 65° C. for 10 minutes), and the capture-associated oligo is optionally isolated from the cleaved capture moiety-cancer marker complex. Following cleavage and optional inactivation or isolation, the capture-associated oligo with the bound oligonucleotide is exposed to an aqueous solution comprising an excess of single nucleotides and the appropriate polymerase, under conditions to allow for polymerization and linear amplification. Step C comprises (i) exposing the capture-associated oligo complex to an aqueous solution comprising the polymerase and an excess of NTP or dNTP and (ii) permitting the polymerase and reactants to create an intermediate duplex comprising a double stranded DNA having a first 5′ end which bears a phage-encoded RNA polymerase recognition site. This reaction continues as the polymerase displaces the doubles stranded nucleic acid, resulting in multiple copies of the capture-associated oligo (Step D). In such an embodiment, the chip-associated oligo will have the same sequence as the capture-associated oligo, and both will be complementary to the linear polymerization products.

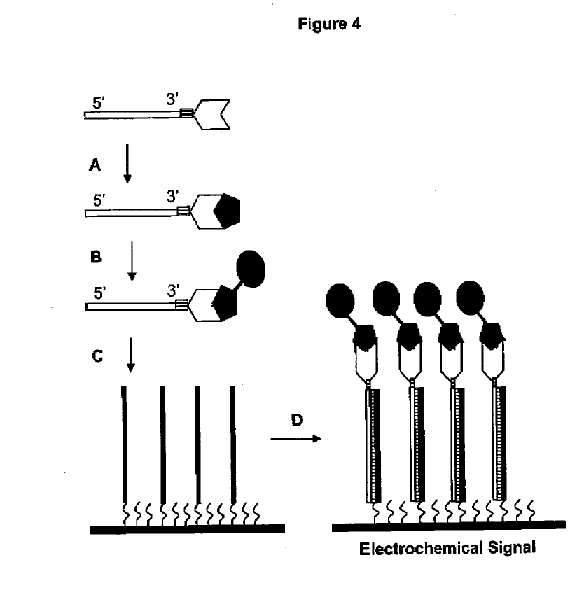

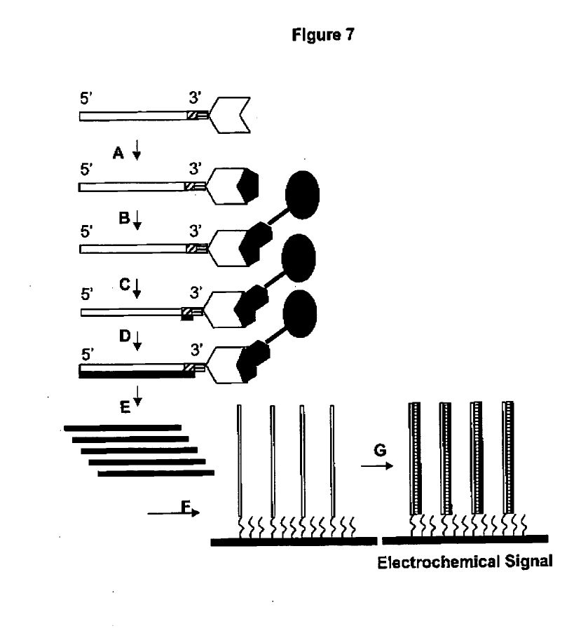

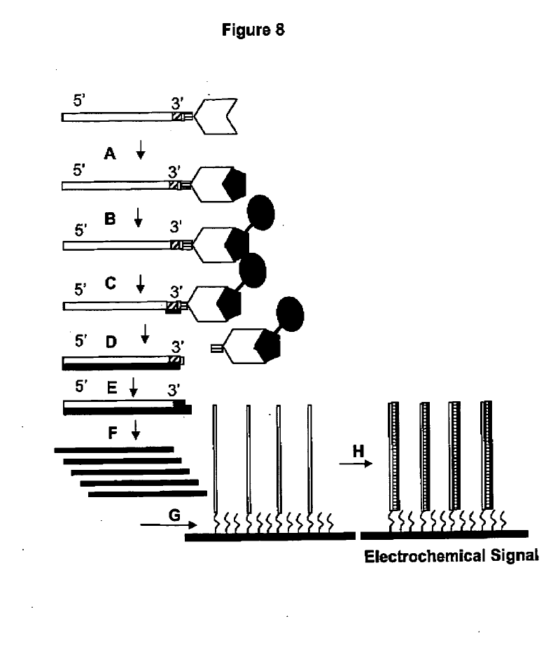

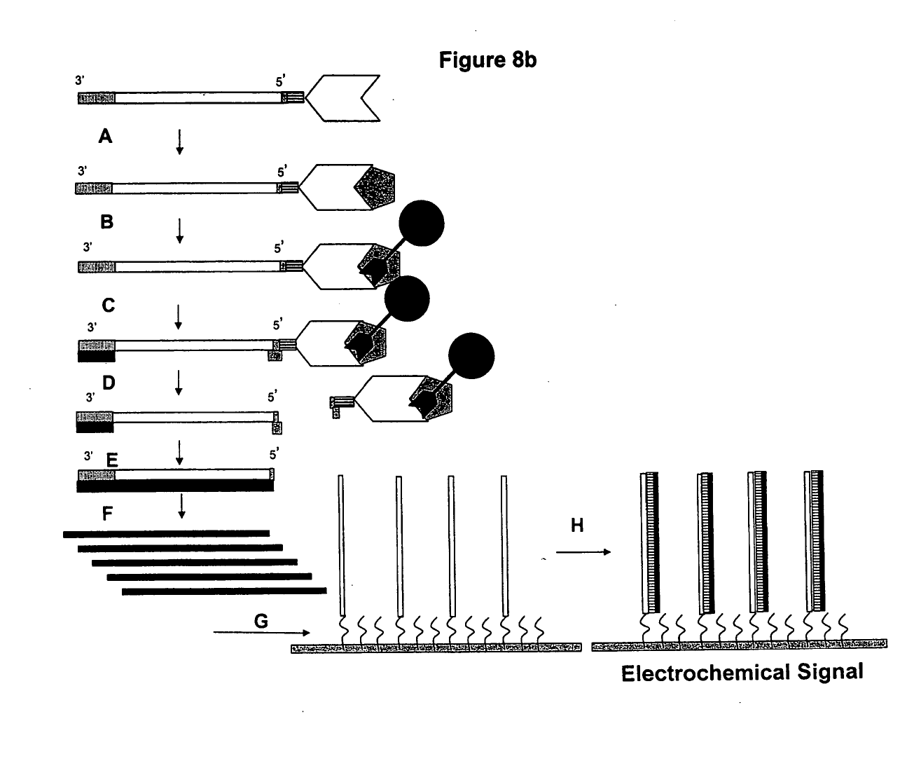

The linear amplification methods using the capture-associated oligo can be combined with any of the described isolation methods of the invention, including those described in FIGS. 3 and 4. For example, FIG. 7 illustrates the embodiment of the invention where the capture-associated oligo is isolated using an immobilized binding partner which binds to the cancer marker, and linear amplification using a polymerase recognition site. FIG. 8 illustrates the embodiment of the invention where the capture-associated oligo is isolated using an antibody that recognizes an epitope specific to the capture moiety/cancer marker complex with cleavage of the capture moiety-cancer marker from the capture-associated oligo prior to linear amplification.

The hybridization reaction between the capture-associated oligos and the chip-associated oligos is typically performed in solution where the metal ion concentration of the buffer is between 0.01 mM to 5 M and a pH range of pH 5 to pH 10. Other components can be added to the buffer to promote hybridization such as dextran sulfate, EDTA, surfactants, etc. The hybridization reaction can be performed at a temperature within the range of 10° C. to 90° C., preferably at a temperature within the range of 25° C. to 60° C., and most preferably at a temperature within the range of 30° C. to 50° C. Alternatively, the temperature is chosen relative to the Tm's of the nucleic acid molecules employed. The reaction is typically performed at an incubation time from 10 seconds to about 12 hours, and preferably an incubation time from 30 seconds to 5 minutes. A variety of hybridization conditions may be used in the present invention, including high, moderate and low stringency conditions; see for example Maniatis et al., Molecular Cloning: A Laboratory Manual, 3rd Edition (2001), hereby incorporated by reference. Persons of ordinary skill in the art will recognize that stringent conditions are sequence-dependent and are dependent upon the totality of the conditions employed. Longer sequences typically hybridize specifically at higher temperatures. Generally, stringent conditions are selected to be about 5-10° C. lower than the thermal melting point (Tm) for the specific sequence at a defined ionic strength pH. Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of destabilizing agents such as formamide. The hybridization conditions may also vary when a non-ionic backbone, i.e., PNA is used, the advantages of using PNA is discussed above. The hybridization reaction can also be controlled electrochemically by applying a potential to the electrodes to speed up the hybridization. Alternatively, the potential can be adjusted to ensure specific hybridization by increasing the stringency of the conditions.

In one embodiment, detection of a hybridization event can be enhanced by the use of an electrochemical hybridization detector. This electrochemical hybridization detector can be, for example, an intercalating agent characterized by a tendency to intercalate specifically to double stranded nucleic acid such as double stranded DNA. These intercalating agents have in their molecules a flat intercalating group such as a phenyl group, which intercalates between the base pairs of the double stranded nucleic acid, therefore binding to the double stranded nucleic acid. Most intercalating agents comprise conjugated electron structures and are therefore optically active; some are commonly used in the quantification or visualization of nucleic acids. Certain intercalating agents exhibit an electrode response, thereby generating or enhancing an electrochemical response. As such, determination of physical change, especially electrochemical change, may serve to detect the intercalating agents bound to a double stranded nucleic acid and so enhance the detection of a hybridization reaction.

Electrochemically active intercalating agents useful in the present invention are, but are not limited to, ethidium, ethidium bromide, acridine, aminoacridine, acridine orange, proflavin, ellipticine, actinomycin D, daunomycin, mitomycin C, HOECHST 33342, HOECHST 33258, aclarubicin, DAPI, Adriamycin, pirarubicin, actinomycin, tris (phenanthroline) zinc salt, tris(phenanthroline) ruthenium salt, tris(phenantroline) cobalt salt, di(phenanthroline) zinc salt, di(phenanthroline) ruthenium salt, di(phenanthroline) cobalt salt, bipyridine platinum salt, terpyridine platinum salt, phenanthroline platinum salt, tris(bipyridyl) zinc salt, tris(bipyridyl) ruthenium salt, tris(bipyridyl) cobalt salt, di(bipyridyl) zinc salt, di(bipyridyl) ruthenium salt, di(bipyridyl) cobalt salt, and the like. Other useful intercalating agents include, inter alia, those listed in Published Japanese Patent Application No. 62-282599. Some of these intercalators contain metal ions and can be considered transition metal complexes. Although the transition metal complexes are not limited to those listed above, complexes which comprise transition metals having oxidation-reduction potentials not lower than or covered by that of nucleic acids are less preferable. The concentration of the intercalator depends on the type of intercalator to be used, but it is typically within the range of 1 ng/ml to 1 mg/ml. Some of these intercalators, specifically Hoechst 33258, has been shown to be a minor-groove binder and specifically binds to double-stranded DNA. The use of such electrochemically active minor groove binders is useful for detection of hybridization in electrochemical detection methods. Thus, in accordance with the present invention, the term “intercalator” is not intended to be limited to those compounds that “intercalate” into the rungs of the DNA ladder structure, but is also intended to include any moiety capable of binding to or with nucleic acids including major and minor groove binding moieties.

Transition metals are those whose atoms have a partial or complete d orbital shell of electrons. Suitable transition metals for use in conjunction with the present invention include, but are not limited to, cadmium (Cd), copper (Cu), cobalt (Co), palladium (Pd), zinc (Zn), iron (Fe), ruthenium (Ru), rhodium (Rh), osmium (Os), rhenium (Re), platinum (Pt), scandium (Sc), titanium (Ti), Vanadium (V), chromium (Cr), manganese (Mn), nickel (Ni), Molybdenum (Mo), technetium (Tc), tungsten (W), and iridium (Ir). That is, the first series of transition metals, the platinum metals (Ru, Rh, Pd, Os, Ir and Pt), along with Fe, Re, W, Mo and Tc, are preferred. Particularly preferred are ruthenium, rhenium, osmium, platinum, cobalt and iron.

The transition metals can be complexed with a variety of ligands, to form suitable transition metal complexes. As will be appreciated by those in the art, the number and nature of the co-ligands will depend on the coordination number of the metal ion. Mono-, di- or polydentate co-ligands may be used at any position. Suitable ligands fall into two categories: ligands, which use nitrogen, oxygen, sulfur, carbon or phosphorus atoms (depending on the metal ion) as the coordination atoms (generally referred to in the literature as sigma (Σ) donors) and organometallic ligands such as metallocene ligands (generally referred to in the literature as pi (π) donors). Suitable nitrogen donating ligands are well known in the art and include, but are not limited to, NH₂; NHR; NRR′; pyridine; pyrazine; isonicotinamide; imidazole; bipyridine and substituted derivatives of bipyridine; terpyridine and substituted derivatives; phenanthrolines, particularly 1,10-phenanthroline (abbreviated phen) and substituted derivatives of phenanthrolines such as 4,7-dimethylphenanthroline and dipyridol[3,2-a:2′,3′-c]phenazine (abbreviated dppz); dipyridophenazine; 1,4,5,8,9,12-hexaazatriphenylene (abbreviated hat); 9,10-phenanthrenequinone diimine (abbreviated phi); 1,4,5,8-tetraazaphenanthrene (abbreviated tap); 1,4,8,11-tetra-azacyclotetradecane (abbreviated cyclam), EDTA, EGTA and isocyanide. Substituted derivatives, including fused derivatives, may also be used. In some embodiments, porphyrins and substituted derivatives of the porphyrin family may be used. See for example, Comprehensive Coordination Chemistry, Ed. Wilkinson et al., Pergammon Press, 1987, Chapters 13.2 (pp 73-98), 21.1 (pp. 813-898) and 21.3 (pp 915-957), all of which are hereby expressly incorporated by reference. Suitable sigma donating ligands using carbon, oxygen, sulfur and phosphorus are known in the art. For example, suitable sigma carbon donors are found in Cotton and Wilkinson, Advanced Organic Chemistry, 5th Ed., John Wiley & Sons (1988), hereby incorporated by reference; see, e.g., page 38. Similarly, suitable oxygen ligands include crown ethers, water and others known in the art. Phosphines and substituted phosphines are also suitable; see, e.g., page 38 of Cotton and Wilkinson. The oxygen, sulfur, phosphorus and nitrogen-donating ligands are attached in such a manner as to allow the heteroatoms to serve as coordination atoms.

Such organometallic ligands include cyclic aromatic compounds such as the cyclopentadienide ion [C₅H₅ (−1)] and various ring substituted and ring fused derivatives, such as the indenylide (−1) ion, that yield a class of bis(cyclopentadieyl) metal compounds, (i.e. the metallocenes); see, e.g., Robins et al., J. Am. Chem. Soc. 104:1882-1893 (1982); and Gassman et al., J. Am. Chem. Soc. 108:4228-4229 (1986), incorporated by reference. Of these, ferrocene [(C₅H₅)₂Fe] and its derivatives are prototypical examples, which have been used in a wide variety of chemical (Connelly et al., Chem. Rev. 96:877-910 (1996), incorporated by reference) and electrochemical (Geiger et al., Advances in Organometallic Chemistry 23:1-93; and Geiger et al., Advances in Organometallic Chemistry 24:87, incorporated by reference) electron transfer or “redox” reactions. Metallocene derivatives of a variety of the first, second and third row transition metals are potential candidates as redox moieties that are covalently attached to the nucleic acid. Other potentially suitable organometallic ligands include cyclic arenes such as benzene, to yield bis(arene) metal compounds and their ring substituted and ring fused derivatives, of which bis(benzene)chromium is a prototypical example. Other acyclic pi-bonded ligands such as the allyl(−1) ion, or butadiene yield potentially suitable organometallic compounds, and all such ligands, in conjunction with other pi-bonded and delta-bonded ligands constitute the general class of organometallic compounds in which there is a metal to carbon bond. Electrochemical studies of various dimers and oligomers of such compounds with bridging organic ligands, and additional non-bridging ligands, as well as with and without metal-metal bonds are potential candidate redox moieties in nucleic acid analysis.

When one or more of the co-ligands is an organometallic ligand, the ligand is generally attached via one of the carbon atoms of the organometallic ligand, although attachment may be via other atoms for heterocyclic ligands. Preferred organometallic ligands include metallocene ligands, including substituted derivatives and the metalloceneophanes (see page 1174 of Cotton and Wilkenson, supra). For example, derivatives of metallocene ligands such as methylcyclopentadienyl, with multiple methyl groups being preferred, such as pentamethylcyclopentadienyl, can be used to increase the stability of the metallocene. In a preferred embodiment, only one of the two metallocene ligands of a metallocene is derivatized.

Alternatively, in some embodiments, the capture-associated universal oligo may be labeled with an electroactive marker. Such electroactive markers can include, but are not limited to, ferrocene derivatives, anthraquinone, silver and silver derivatives, gold and gold derivatives, osmium and osmium derivatives, ruthinium and ruthinium derivatives, cobalt and cobalt derivatives and the like.

When traditional microarray technology is employed using fluorescence to detect a hybridization event between the capture-associated oligo and the chip-associated oligo, an optical detection device may be used for detection (see, e.g., U.S. Pat. Nos. 5,578,832; 5,631,734; 5,834,758; 6,025,601; 6,141,096 and 6,252,236, the complete disclosures of which are incorporated herein by reference).

Such devices generally employ a scanning device which rapidly sweeps an activation radiation beam or spot across the surface of the chip substrate. Optical detection devices also include focusing optics for focusing the excitation radiation onto the surface of the substrate in a sufficiently small area to provide high resolution of features on the substrate, while simultaneously providing a wide scanning field. An image is obtained by detecting the electromagnetic radiation emitted by the labels on the sample when the labels are illuminated. In some embodiments, fluorescent emissions are gathered by the focusing optics and detected to generate an image of the fluorescence on the substrate surface. The optical detection devices may further employ confocal detection systems to reduce or eliminate unwanted signals from structures above and below the plane of focus of the excitation radiation, as well as autofocus systems to focus both the activation radiation on the substrate surface and the emitted radiation from the surface. Generally, the excitation radiation and response emission have different wavelengths.

In operation, optical detection devices include one or more sources of excitation radiation. Typically, these source(s) are immobilized or stationary point light sources, e.g., lasers such as argon, helium-neon, diode, dye, titanium sapphire, frequency-doubled diode pumped Nd:YAG and krypton. Typically, the excitation source illuminates the sample with an excitation wavelength that is within the visible spectrum, but other wavelengths (i.e., near ultraviolet or near infrared spectrum) may be used depending on the application. In some cases, the label is excited with electromagnetic radiation having a wavelength at or near the absorption maximum of the species of label used. Exciting the label at such a wavelength produces the maximum number of photons emitted. For example, if fluorescein (absorption maximum of 488 nm) is used as a label, an excitation radiation having a wavelength of about 488 nm would induce the strongest emission from the labels.

The excitation radiation from the point source is directed at a movable radiation direction system which rapidly scans the excitation radiation beam back and forth across the surface of the substrate. A variety of devices may be employed to generate the sweeping motion of the excitation radiation. For example, resonant scanner or rotating polygons, may be employed to direct the excitation radiation in this sweeping fashion. Generally, however, galvanometer devices are preferred as the scanning system. In addition, an optical train may be employed between the activation source and the galvanometer mirror to assist in directing, focusing or filtering the radiation directed at and reflected from the galvanometer mirror.

The galvanometers employed in such optical detection devices and systems of the present invention typically sweep a scanning spot across the substrate surface at an oscillating frequency that is typically greater than 30 Hz. The objective lens is preferably selected to provide high resolution, as determined by the focused spot size, while still allowing a wide scanning field.

As the activation radiation spot is swept across the surface of the substrate, it activates fluorescent groups on any capture-associated universal oligos that have bound to the chip-associated universal oligos. The activated groups emit a response radiation or emission which is then collected by the objective lens and directed back through the optical train via the servo mounted mirror. In order to avoid the detrimental effects of reflected excitation radiation upon the detection of the fluorescence, dichroic mirrors or beam splitters may be included in the optical train. These dichroic beam splitters or mirrors are reflective to radiation in the wavelength of the excitation radiation while transmissive to radiation in the wavelength of the response radiation. For example, where an Argon laser is used as the point energy source, it will typically generate activation radiation having a wavelength of about 488 nm. Fluorescence emitted from an activated fluorescein moiety on the other hand will typically have a wavelength between about 515 and 545 nm. As such, dichroic mirrors may be included which transmit light having a wavelength greater than 515 nm while reflecting light of shorter wavelengths. This effectively separates the excitation radiation reflected from the surface of the substrate from the response radiation emitted from the surface of the substrate. Additional dichroic mirrors may be used to separate signals from label groups having different response radiation wavelengths, thereby allowing simultaneous detection of multiple fluorescent indicators.

Following separation of the response radiation from the reflected excitation radiation, the response radiation or fluorescence can be directed at a detector, e.g., a photomultiplier tube, to measure the level of response radiation and record that level as a function of the position on the substrate from which that radiation originated. Typically, the response radiation is focused upon the detector through a spatial filter such as a confocal pinhole. Spatial filters reduce or eliminate unwanted signals from structures above and below the plane of focus of the excitation radiation. Additionally, the device may incorporate a bandpass filter between the dichroic mirror and the detector to further restrict the wavelength of radiation that is delivered to the detector.

In certain preferred embodiments, the polymerization product comprises RNA sequences (e.g., a T7 transcription product) that are complementary to the chip-associated oligo. Thus, hybrids resulting from hybridization between the chip-associated oligo and the polymerization products will be DNA:RNA duplexes (when the chip-associated oligos are DNA) or RNA:RNA duplexes (when the chip-associated oligos are RNA). The resulting hybrids can be detected by an antibody reagent capable of binding to the DNA:RNA or RNA:RNA duplexes. A variety of protocols and reagent combinations can be employed in order to carry out this embodiment of the present method.

Detection of a DNA:RNA or RNA:RNA hybrid-recognizing antibody reagent can be accomplished in any convenient manner. In a preferred embodiment, the antibody reagent can be labeled with a moiety such as an enzymatically active group, a fluorescer, a chromophore, a luminescer, a specifically bindable ligand, a electrochemically detectable molecule/moiety, a radioisotope or the like, with the nonradioisotopic labels being especially preferred. The labeled antibody reagent which becomes bound to resulting immobilized hybrid duplexes can be readily separated from that which does not become so bound.

It should be understood that by the expressions “RNA,” “DNA,” “RNA nucleotide sequence,” “DNA nucleotide sequence,” or similar designations herein, it is not implied or intended that all nucleotides comprised in the nucleic acids be ribonucleotides or 2′-deoxyribonucleotides. The fundamental feature of an RNA or DNA capture-associated oligo or chip-associated oligo for purposes of the present invention is that it be of such character to be detected by antibodies to DNA:RNA hybrids, where individual single strands are not bound by such an antibody and DNA:DNA hybrids are not bound by such an antibody. One or more of the 2′-positions on the nucleotides comprised in the nucleic acids can be chemically modified, provided the antibody binding characteristics necessary for performance of the present assay are maintained to a substantial degree. Likewise, in addition or alternatively to such limited 2′-deoxy modification, a chip-associated oligo, capture-associated oligo or polymerization product can have in general any other modification along its ribose phosphate backbone provided there is no substantial interference with the specificity of the antibody to the DNA:RNA or RNA:RNA hybridization product compared to individual single strands or to DNA:DNA hybrids.

Where such modifications exist in a nucleic acid, the immunogen used to raise the antibody reagent would preferably comprise one strand having substantially corresponding modifications and the other strand being substantially unmodified RNA or DNA, depending on whether sample RNA or DNA was intended to be detected. Preferably, the modified strand in the immunogen would be identical to the modified strand in an RNA or DNA oligo. An example of an immunogen is the hybrid poly(2′-O-methyladenylic acid):poly(2′-deoxythymidylic acid). Another would be poly(2′-O-ethylinosinic acid):poly(ribocytidylic acid). The following are further examples of modified nucleotides which could be comprised in a modified nucleic acid: 2′-O-methylribonucleotide, 2′-O-ethylribonucleotide, 2′-azidodeoxyribonucleotide, 2′-chlorodeoxyribonucleotide, 2′-O-acetylribonucleotide, and the phosphorothiolates or methylphosphonates of ribonucleotides or deoxyribonucleotides. Modified nucleotides can appear in nucleic acids as a result of introduction during enzymic synthesis of the nucleic acid from a template. For example, adenosine 5′-O-(1-thiotriphosphate) (ATPαS) and dATPαS are substrates for DNA dependent RNA polymerases and DNA polymerases, respectively. Alternatively, the chemical modification can be introduced after the nucleic acid has been prepared. For example, an RNA oligo can be 2′-O-acetylated with acetic anhydride under mild conditions in an aqueous solvent (see, e.g., Steward, D. L. et al, Biochim. Biophys. Acta 262:227 (1972)).

A detection antibody reagent of certain preferred embodiments of the invention is typically characterized by its ability to bind the DNA:RNA hybrids formed to the significant exclusion of single stranded polynucleotides or to DNA:DNA hybrids. The detection antibody reagent can consist of whole antibodies, antibody fragments, polyfunctional antibody aggregates, or in general any substance comprising one or more specific binding sites from an antibody for DNA:RNA. When in the form of whole antibody, it can belong to any of the classes and subclasses of known immunoglobulins, e.g., IgG, IgM, and so forth. Any fragment of any such antibody which retains specific binding affinity for the hybridized nucleic acid can also be employed; for instance, the fragments of IgG conventionally known as Fab, F(ab′), and F(ab′)₂. In addition, aggregates, polymers, derivatives and conjugates of immunoglobulins or their fragments can be used where appropriate.

The immunoglobulin source for the antibody reagent can be obtained in any available manner such as conventional antiserum and monoclonal techniques. Antiserum can be obtained by well-established techniques involving immunization of an animal, such as a mouse, rabbit, guinea pig or goat, with an appropriate immunogen. The immunoglobulins can also be obtained by somatic cell hybridization techniques, such resulting in what are commonly referred to as monoclonal antibodies, also involving the use of an appropriate immunogen.

Immunogens for stimulating antibodies specific for DNA:RNA hybrids can comprise homopolymeric or heteropolymeric polynucleotide duplexes. Among the possible homopolymer duplexes, particularly preferred is poly(rA).poly(dT) (see, e.g., Kitagawa and Stollar Mol. Immunol. 19:413 (1982)). However, in general, heteropolymer duplexes will be preferably used and can be prepared in a variety of ways, including transcription of φX174 virion DNA with RNA polymerase (see, e.g., Nakazato, Biochem. 19:2835 (1980)). The selected RNA:DNA duplexes are typically adsorbed to a methylated protein, or otherwise linked to a conventional immunogenic carrier material, such as bovine serum albumin, and injected into the desired host animal (see e.g., U.S. Pat. No. 5,200,313, and Stollar, Meth. Enzymol., 70:70 (1980)).

In certain alternative embodiments, it may be preferred to use chip-associated oligos that comprise RNA and, as such, RNA:RNA duplexes will be formed during the hybridization process. Antibodies to RNA:RNA duplexes can substituted for the antibodies to DNA:RNA duplexes described herein without deviating from the present invention. Antibodies to RNA:RNA duplexes can, for example without limitation, be raised against double stranded RNAs from viruses such as reovirus or Fiji disease virus which infects sugar cane, among others. Also, homopolymer duplexes such as poly(rI).poly(rC) or poly(rA) poly(rU), among others, can be employed as above.

In certain preferred embodiments, the “chip associated oligo” will not be immobilized but instead is contacted with the complexed conjugated oligo in solution. The double stranded molecule resulting from a hybridization event, can be captured (i.e., immobilized) by a variety of methods including, inter alia, by immobilized capture antibodies. The methods for such capture are similar to the forgoing examples regarding antibody labeling of the immobilized complex.

The binding of the detection antibody reagent to the hybridized duplex according to the present method can be detected by any convenient technique. Advantageously, the antibody reagent will itself be labeled with a detectable chemical group. Such detectable chemical group can be any material having a detectable physical or chemical property. Such materials have been well-developed in the field of immunoassays and in general most any label useful in such methods can be applied to the present invention. Particularly useful are, inter alia: enzymatically active groups, such as enzymes (see, e.g., Clin. Chem., 22:1243 (1976), U.S. Pat. No. 31,006 and UK Pat. 2,019,408), enzyme substrates (see, e.g., U.S. Pat. No. 4,492,751, cofactors (see, e.g., U.S. Pat. Nos. 4,230,797 and 4,238,565), and enzyme inhibitors (see, e.g., U.S. Pat. No. 4,134,792); fluorescers (see, e.g., Clin. Chem., 25:353 (1979)); chromophores; luminescers such as chemiluminescers and bioluminescers (see, e.g., U.S. Pat. No. 4,380,580); specifically bindable ligands such as biotin (see, e.g., European Pat. Spec. 63,879) or a hapten (see, e.g., PCT Publ. 83-2286); electrochemically detectable reagents/moieties (see, e.g., U.S. Pat. Nos. 5,776,672; 5,972,692 and U.S. Pat. Pub. 2002/01467162004/0063126) and radioisotopes such as ³H, ³⁵S, ³²P, ¹²⁵I, and ¹⁴C. Such labels and labeling pairs are detected on the basis of their own physical properties (e.g., fluorescers, chromophores and radioisotopes) or their reactive or binding properties (e.g., enzymes, substrates, cofactors and inhibitors). For example, a cofactor-labeled antibody can be detected by adding the enzyme for which the label is a cofactor and a substrate for the enzyme. A hapten or ligand (e.g., biotin) labeled antibody can be detected by adding an antibody to the hapten or a protein (e.g., avidin) which binds the ligand, tagged with a detectable molecule. Such detectable molecules can be some molecule with a measurable physical property (e.g., fluorescence or absorbance) or a participant in an enzyme reaction (e.g., supra). For example, one can use an enzyme which acts upon a substrate to generate a product with a measurable physical property. Examples of the latter include, but are not limited to, β-galactosidase, alkaline phosphatase and peroxidase. Other labeling schemes will be evident to one of ordinary skill in the art.

Alternatively, the detection antibody reagent can be detected based on a native property such as its own antigenicity. A labeled anti-(antibody) antibody will bind to the primary antibody reagent where the label for the second antibody is a conventional label as above. Further, antibody can be detected by complement fixation or the use of labeled protein A, protein G, as well as other techniques known in the art for detecting antibodies.

Where the detection antibody reagent is labeled, as is preferred, the labeling moiety and the antibody reagent are associated or linked to one another by direct chemical linkage such as involving covalent bonds, or by indirect linkage such as by incorporation of the label in a microcapsule or liposome which is in turn linked to the antibody. Labeling techniques are well-known in the art and any convenient method can be used in the present invention.

The present invention also contemplates the use of kits to detect cancer markers. The kits may include capture-associated universal oligos and immobilized binding partners to the capture moieties. The kit also may include a universal oligo chip comprising a plurality of chip-associated universal oligos. In preferred embodiments, the kit includes a primer for linear amplification of the capture-associated universal oligo and a T7 or other polymerase in an appropriate buffer. In addition, the kit can include a label for fluorescent detection, an antibody for, e.g., the detection of RNA:RNA or DNA:RNA hybrids, or an electrochemical hybridization indicator for electrochemical detection.

### EXAMPLE I

### Preparation of Monoclonal Antibodies

A peptide corresponding to amino acid residues in a desired cancer marker antigen is synthesized with a peptide synthesizer (Applied Biosystems) according to methods known in the art. The peptide emulsified with Freund's complete adjuvant is used as an immunogen. And administered to mice by footpad injection for primary immunization (day 0). The booster immunization is performed four times or more in total. The final immunization is carried out by the same procedure two days before the collection of lymph node cells. The lymph node cells collected from each immunized mouse and mouse myeloma cells are mixed at a ratio of 5:1. Hybridomas are prepared by cell fusion using polyethylene glycol 4000 or polyethylene glycol 1500 (GIBCO) as a fusing agent. The lymph node cells of the mouse are fused with mouse myeloma PAI cells (JCR No. B0113; Res. Disclosure Vol. 217, p. 155, 1982), and the resulting hybridomas are selected by culturing the fused cells in an ASF104 medium (Ajinomoto Co. Inc.) containing HAT supplemented with 10% fetal calf serum (FCS) and aminopterin. The reactivity of the culture supernatant of each hybridoma clone to the is measured by ELISA.

Screening by ELISA is performed by adding the cancer marker into each well of a 96-well ELISA microplate (Corning Costar Co.). The plate is incubated at room temperature for 2 hours for the adsorption of the cancer marker onto the microplate. The supernatants are discarded and then the blocking reagent (200 μl; phosphate buffer containing 3% BSA) is added into each well. The plate is incubated at room temperature for 2 hours to block free sites on the microplate. Each well is washed three times with 200 μl of phosphate buffer containing 0.1% Tween 20. Supernatant (100 μl) from each hybridoma culture is added into each well of the plate, and the reaction is allowed to proceed for 40 minutes. Each well is then washed three times with 200 μl of phosphate buffer containing 0.1% Tween 20. In the next step, biotin-labeled sheep anti-mouse immunoglobulin antibody (50 μl; Amersham) is added to the wells and the plates are incubated at room temperature for 1 hour.

The microplate is washed with phosphate buffer containing 0.1% Tween 20. A solution of streptavidin-β-galactosidase (50 μl; Gibco-BRL), diluted 1000 times with a solution (pH 7.0) containing 20 mM HEPES, 0.5M NaCl and bovine serum albumin (BSA, 1 mg/ml), is added into each well. The plate is then incubated at room temperature for 30 minutes. The microplate is then washed with phosphate buffer containing 0.1% Tween 20. A solution of 1% 4-Methyl-umbelliferyl-β-D-galactoside (50 μl; Sigma) in a phosphate buffer (pH 7.0) containing 100 mM NaCl, 1 mM MgCl₂ and 1 mg/ml BSA, is added into each well. The plate is incubated at room temperature for 10 minutes. 1M Na₂CO₃ (100 μl) is added into each well to stop the reaction. Fluorescence intensity is measured in a Fluoroscan II Microplate Fluorometer (Flow Laboratories Inc.) at a wavelength of 460 nm (excitation wavelength: 355 nm).

### EXAMPLE II

### Preparation of DNA-Antibody Conjugates

A capture-associated universal oligonucleotide can be prepared on a solid support that has been treated with 3-amino-1,2-propanediol in order to introduce the 3′ amino group with an automated DNA synthesizer (e.g., 3400 DNA synthesizer, Applied Biosystems). Typical cleavage and purification steps are employed to obtain the modified universal oligonucleotide. The universal oligonucleotide is then incubated with N-succinimidyl 3-(2-pyridyldithio)propionate in PBS at a molar ratio between 1:30 to 1:35 for 30 minutes at room temperature. Dithiothreitol is typically added to this solution, resulting in a final concentration of 10 mM for 5 minutes. The universal oligonucleotide is then purified and recovered by applying this reaction mixture to a PBS equilibrated Sepharose column, washing the column several times, and eluting the universal oligonucleotide in a 0.6M NaCl phosphate buffer.

A monoclonal antibody is incubated with γ-maleimidobutyric acid-N-hydroxysuccinimide ester in PBS at a molar ratio of between 1:15 and 1:20 for 30 minutes at room temperature. The maleimide derivatized antibody can then be purified by column chromatography.

The conjugation of the monoclonal antibody and the oligonucleotide is typically achieved by mixing the maleimide derivatized antibody and the sulphydryl containing oligonucleotide in a molar ratio between 1:10 and 1:16 and incubated overnight at 4° C. The resulting conjugates are purified by precipitation with a 50% saturated solution of (NH₄)₂SO₄ and extensive washing in the same (NH₄)₂SO₄ solution. Residual (NH₄)₂SO₄ can then be removed by dissolving the precipitate in PBS and gel filtration.

### EXAMPLE III

### Immobilization of Nucleic Acid Probe to a Platinum Electrode Surface

A platinum electrode is exposed to a high temperature to air-oxidize the surface of the electrode. The oxidized electrode is treated with cyanogen bromide (CNBr) to activate the oxide layer. The nucleic acid is attached to the electrode by contacting the electrode in a solution of single stranded nucleic acid. The single stranded nucleic acid is obtained by commonly employed means including, but not limited to, either standard oligonucleotide synthesis techniques or by thermal denaturation of a double stranded nucleic acid molecule.

Alternatively, a custom synthesized oligonucleotide containing a thiol group at the 5′ or the 3′ end is spotted on a gold electrode. This procedure involves placing approximately 100 nL of the probe solution containing the oligonucleotide probe (5 μmol/L), 400 mmol/L sodium chloride, and 0.1 mmol/L HCl, on the electrode and then keeping the electrode at room temperature for 1 h thereby resulting in the probes be immobilized onto the gold surface via a thiol moiety. Unattached probes are removed by washing the electrode with distilled water.

### EXAMPLE IV

### Binding of Cancer Marker and Removal of Excess Conjugate

A sample is obtained from a patient suspected of having cancer is diluted in PBS/Tween20. An oligonucleotide conjugated to an anti-cancer marker antibody (the procedure for conjugation is described in Example II) is contacted with the diluted sample by adding a one-third volume of bovine serum albumin (12% [wt/vol] in PBS) and 2 μg of antibody-nucleic acid conjugate. The resulting reaction is incubated at room temperature for 30 minutes.

Unbound antibody-nucleic acid conjugate is removed by magnetic microparticle depletion. Briefly, magnetic microparticles are coated with the epitope recognized by the anti-cancer marker antibody. The epitope-coated magnetic beads are added to the reaction mixture, in a PBS buffer supplemented with 0.5% BSA and 2 mM EDTA, and incubated at 4° C. for 30 minutes. Only those antibody-nucleic acid conjugates that have not bound to cancer marker in the sample are available to bind to the immobilized epitope. The magnetically labeled excess conjugate is separated from the reaction mixture by adding the mixture to a column packed with lattice-type matrix and applying a magnetic field. Such separation devices are known in the art (e.g., MACS® Columns, Miltenyi Biotec). The magnetically labeled antibody-nucleic acid conjugate is retained on the column; the target bound conjugates passing through the column and is available for detection.

### EXAMPLE V

### Cleavage of the Antibody from the Nucleic Acid Strand

Following the isolation of the target bound conjugates, it may be desirable in some instances to remove the antibody and the cancer marker from the nucleic acid prior to hybridization. This is accomplished by performing a cleavage reaction to cleave the nucleic acid between the portion of the nucleic acid that will hybridize to the electrode immobilized nucleic acid molecule and the conjugated antibody.

An oligonucleotide is synthesized as described in Example II with a “G-G-C-C” sequence between the conjugated antibody and the portion of the oligonucleotide that will hybridize to the electrode immobilized nucleic acid molecule. The restriction endonuclease, HaeIII (New England Biolabs), has been shown to cleave single stranded DNA at this specific sequence (Horiuchi & Zinder, 1975). The cleavage reaction is performed by mixing the HaeIII enzyme with the antibody-nucleic acid conjugate in a buffer containing 10 mM Tris-HCl, 50 mM NaCl, 10 mM MgCl₂, 1 mM dithiothreitol, pH 7.9, and incubating at 37° C. for 30 minutes. The HaeIII enzyme is heat inactivated at 80° C. for 20 minutes. The cleaved nucleic acid molecules are separated from the antibody-antigen complex by standard techniques such as ethanol precipitation. Briefly, add 2.5-3 volumes of 95% ethanol/0.12 M sodium acetate to the DNA sample contained in a 1.5 ml microcentrifuge tube, invert to mix, and incubate in an ice-water bath for 10 minutes. The resulting mixture is centrifuged at 12,000 rpm in a microcentrifuge for 15 min at 4° C., decant the supernatant, and drain inverted on a paper towel. Ethanol (80%) (corresponding to about two volume of the original sample) is added and the reaction mixture is incubated at room temperature for 5-10 min followed by centrifugation for 5 min. The supernatant is then decanted. The sample is air dried (or alternatively lyophilized) and the pellet of DNA resuspended in 10 mM Tris-HCl, pH 7.6-8.0, 0.1 mM EDTA. For hybridization reactions, the nucleic acid is resuspended in SSC solution.

In an alternative cleavage method, photocleavage is performed. In doing so, an oligonucleotide is synthesized as described in Example II with a photocleavable nucleotide inserted into the sequence. This can be accomplished by using a photocleavable phosphoramidite during the synthesis of the oligonucleotide (Glen Research). The cleavage reaction is essentially performed by exposing the oligonucleotide-antibody conjugate to a source of ultraviolet (UV) light. The cleaved nucleic acid molecules are separated from the antibody-antigen complex by standard techniques such as ethanol precipitation, membrane filtration, or if the antibody-antigen complex is immobilized, by centrifugation, etc.

### EXAMPLE VI

### Hybridization of Nucleic Acid Molecules to the Electrode-Immobilized Nucleic Acid Molecules

The hybridization and detection reaction is carried out as follows. Single stranded nucleic acid in 2×SSC solution (300 mmol/L NaCl, 30 mmol/L trisodium citrate) are contacted with the probes immobilized on the electrode. The hybridization reaction is carried out at a temperature that permits specific hybridization of the two nucleic acid molecules. The temperature of the hybridization reaction is performed is determined using the equation for calculating the melting temperature of an oligonucleotide. It is possible to shorten the incubation time of this hybridization reaction by applying 0.1 V to the electrode. Using this procedure it may be possible to shorten the incubation time to 10 minutes.

To enhance detection, an electrochemical hybridization indicator, such as a minor groove binder is added. Briefly, a solution containing 50 μmol/L Hoechst 33258 (WAKO Pure Chemicals Industries, Ltd.) and 100 mmol/L NaCl is added before, during, or after hybridization. If the Hoechst 33258 is added after the hybridization reaction, then a further incubation of 5 minutes may be necessary. The electrochemical analysis is carried out with an electrochemical analyzer (Model BAS-100B) and software from Bioanalytical Systems, Inc. or the Genelyzer System from Toshiba Corporation. The cyclic voltammetry is typically carried out at 300 mV/s and 25° C., and the potential sweep range from −100 to 900 mV.

### EXAMPLE VII

### Binding of Cancer Marker and Alternative Method of Removal of Excess Conjugate

A sample is obtained from a patient suspected of having cancer is diluted in PBS/Tween20. An oligonucleotide conjugated to an anti-cancer marker antibody (the procedure for conjugation is described in Example II) is contacted with the diluted sample by adding a one-third volume of bovine serum albumin (12% [wt/vol] in PBS) and 2 μg of antibody-nucleic acid conjugate. The resulting reaction is incubated at room temperature for 30 minutes.

Unbound antibody-nucleic acid conjugate is removed by magnetic microparticle depletion. Briefly, magnetic microparticles are coated with a second anti-cancer marker antibody, specific to another region (epitope) of the same cancer marker (binding partner, antigen) to be detected. The second antibody-coated magnetic beads are added to the reaction mixture, in a PBS buffer supplemented with 0.5% BSA and 2 mM EDTA, and incubated at 4° C. for 30 minutes. Only those antibody-nucleic acid conjugates that have bound to cancer marker in the sample are available to bind to the magnetic particle immobilized second anti-cancer marker antibody, specific to another region (epitope) of the same cancer marker to be detected. The magnetically labeled conjugate is separated from the reaction mixture by adding the mixture to a column packed with lattice-type matrix and applying a magnetic field. Such separation devices are known in the art (e.g., MACS® Columns, Miltenyi Biotec). The magnetically labeled second antibody-nucleic acid conjugate that is bound to the cancer marker is retained on the column. The antibody-nucleic acid conjugate that is not bound to the cancer marker will pass through the column.

Subsequently cleavage of the nucleic acid from the magnetically labeled second antibody-nucleic acid conjugate that is bound to the cancer marker is performed as described in Example V. This cleavage can be achieved by other approaches, described earlier in this invention. The cleavage products are then subjected to electrochemical detection.

### EXAMPLE VIII

### Binding of Cancer Marker without Direct Interaction with the Causative Agent

A sample is obtained from a patient suspected of having cancer. The sample is diluted in a diluent such as PBS/tween20. An oligonucleotide conjugated to a cancer marker-specific antigen is incubated with the diluted sample by adding a one third volume of bovine serum albumin (12% [wt/vol] in PBS) and 2 ug of the oligo nucleotide-antigen conjugate. Unbound nucleic acid-antigen complex is removed by magnetic microparticle-antibody affinity depletion. Briefly, magnetic micro-particles are coated with an antibody affinity reagent such as Protein A, Protein G or anti-class antibody which captures antibodies from the sample, a portion of which may be cancer marker antigen specific and bound to the antigen-oligo conjugate. The coated magnetic beads are added to the reaction mixture, in a PBS buffer supplemented with 0.5% BSA and 2 mm EDTA, and incubated at 40 C for 30 minutes. Antibodies in the sample will be immobilized on the magnetic beads, only anti-cancer marker antibodies will contain the oligo-antigen conjugate. The magnetically labeled antibody affinity reagent, along with additional binding partners (oligo-antigen complexes) are separated from the rest of the sample and extensively washed with PBS/Tween20. Such separation techniques are known in the art (e.g., MACS Columns, Miltenyi Biotec). Subsequent release of the oligo from the antigen is performed as described in Example V or by other approaches described herein.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. §112, ¶6.

Each reference cited herein is incorporated by reference in its entire

TABLE 1
Examples of cancer antigens and their associated cancers

| Common name(s) of Cancer Antigen | Some associated tumors/cancers | Select refs |
|---|---|---|
| Prostate-specific antigen (PSA) | Prostate cancer | ^{1, 2} |
| Prostatic acid phosphatase (PAP) | Prostate cancer, testicular cancer, leukemia, and | ^{3, 4} |
| | non-Hodgkin's lymphoma | |
| CA 125 | Ovary, uterus, cervix, pancreas, liver, colon, breast, | ^{5, 6, 7, 8} |
| | lung, gall bladder and digestive cancer | |
| Carcinoembryonic antigen (CEA) | Colorectal cancer and several other cancers | ^{9, 10} |
| Alpha-fetoprotein (AFP) | Liver cancer or cancer of the ovary or testicle | ^{11, 12} |
| Human chorionic gonadotropin (HCG) | Cancer in the testis, ovary, liver, stomach, pancreas, | ^{13, 14, 15} |
| | and lung | |
| CA 19-9 | Cancers of the colon, stomach, bile duct, and pancreas | ^{16, 17} |
| CA 15-3 | Advanced breast cancer, cancers of the ovary, lung, and | ^{18, 19} |
| | prostate | |
| CA 27-29 | Advanced breast cancer, colon, stomach, kidney, lung, | ¹⁹ |
| | ovary, pancreas, uterus, liver cancer | |
| lactate dehydrogenase (LDH) | Elevated LDH levels present in many cancers | ^{18, 20, 21, 22, 23} |
| Neuron-specific enolase (NSE) | Neuroblastoma, small cell lung cancer | ^{22, 24, 25} |
| Bcl-2 proto-oncogene | Human lymphoma | ^{26, 27, 28} |
| BCA-225 | Breast carcinoma | ^{29, 30} |
| c-Met | Variety of human carcinomas and sarcomas | ^{31, 32} |
| CA 15-3 | Most primary and metastatic breast tumors, and most | ¹⁰ |
| | tumors of the ovary | |
| Cathepsin D | Breast carcinoma/hepatoma, carcinomas and sarcomas | ^{33, 34} |
| Cytokeratin (HMW) | Squamous carcinomas and prostate tumors | ^{35, 36} |
| Human epithelial cadherin (E-cadherin, uvomorulin) | Reduction/loss as a metastatic marker | ^{37, 38, 39} |
| E2F-1 | Overexpressed in lung, breast or bladder carcinomas | ^{40, 41} |
| EGF receptor | Breast carcinoma | ⁴² |
| EphB4 receptor and ephrin-B2 ligand | Primary metastatic HNSCC, colon and breast carcinoma, | ^{43, 44} |
| | endometrial cancer | |
| Estrogen receptor and progesterone receptor | Breast carcinoma | ^{45, 46} |
| EZH2, also known as ENX-1 | Prostate cancer, aggressive breast cancer | ^{47, 48} |
| FHIT (Fragile Histidine Triad) | Tumor suppressor. Cancers of the lung, cervix, breast, | ^{49, 50} |
| | colon, stomach, and pancreas | |
| GCDFP-15 | Highly specific marker for breast cancer; differential | ^{51, 52} |
| | diagnosis of metastatic breast cancers | |
| HER2 (c-erbB-2) | Breast cancer | ^{53, 54} |
| HER4 (c-erbB-4) | Squamous cell carcinoma of the head and neck and | ^{55, 56, 57} |
| | hepatocellular carcinoma | |
| Ki-67 | Breast carcinoma | ^{58, 59} |
| Metallothioneins | Hepatocellular carcinoma | ^{60, 61} |
| MTA1 | Metastasis marker | ^{62, 63} |
| Mucin 1 | Metastasis marker | ^{64, 65} |
| NY-ESO-1, a member of the CT (cancer/testis) family | Melanoma, bladder cancer and some sarcomas | ^{66, 67} |
| p53 (mutant) | Tumor suppressor; mutant found in most malignant tumors | ^{68, 69, 70, 78} |
| PRL-3 | Metastatic colorectal cancer | ^{71, 72} |
| Trefoil peptide pS2 | Breast carcinoma | ^{73, 74} |
| Retinoblastoma gene (Rb) | Numerous tumors (retinoblastoma, breast carcinoma, | ^{75, 76, 77, 78} |
| | prostate cancer, lung and bladder cancer) | |
| SKP2 (S-phase Kinase-associated Protein 2) | Most tumors | ^{79, 80, 81} |
| Smad 3 | Down-regulated in high-grade breast cancer | ⁸² |
| STAT3 (Signal Transducers and Activators of Transcription3) | Breast, bronchioloalveolar carcinoma and prostate | ^{83, 84, 85} |
| | carcinoma | |
| TAG 72 (CA 72.4) | Colorectal, gastric, pancreatic, ovarian, endometrial, | ^{86, 87, 88} |
| | mammary and NSCL cancer) | |
| Tenascin | Tumor progression marker | ^{89, 90} |
| Tissue inhibitors of metalloproteinase (TIMPs) | Metastasis marker (downregulation) | ^{91, 92} |
| Topo II-alpha | Tumor progression marker | ^{93, 94, 95} |
| Urokinase-type plasminogen activator receptor (uPAR) | Endometrial carcinoma, breast cancer or gastric cancer | ^{96, 97} |
| Platelet-derived endothelial cell growth factor (PD-ECGF) | Overexpression marker for neovascularization of tumors, | ^{98, 99} |
| | tumor cell invasion and metastasis | |
| Melanocortin-1 receptor (MC1R) mutant variants | Melanoma | ^{100, 101} |
| AMACR (Alpha-methylacyl-CoA-racemase (P504S)) | Prostate cancer, urothelial carcinoma | ^{102, 103} |
| p63 | Prostate cancer | ^{104, 105} |
| CD117 (c-Kit) receptor | Erythroleukemia; small cell lung cancer | ^{106, 107} |
| COX-2 | Colon carcinoma | ^{108, 109} |
| Ezrin | Choriocarcinoma and Raji Burkitts lymphoma | ^{110, 111} |
| FLI-1 protein | Angiosarcomas | ¹¹² |
| Galectin-3, Galectin-7 | Hypopharyngeal Cancer, thyroid cancer | ¹¹³ |
| Glutaminyl cyclase | Melanoma | ¹¹⁴ |
| MAGE-A1/2, MAGE-A3/4, MAGE-A12 | Squamous cell carcinoma, thyroid cancer | ¹¹⁵ |
| Mesothelin 1 | Mesothelioma, pancreatic cancer, ovarian cancer | ^{116, 117} |
| Prostate and testis expressed protein (PATE), Kallikriens | Prostate cancer | ^{118, 119} |
| Pax5 | Bladder cancer, multiple myeloma, B-cell lymphoma | ¹²⁰ |
| PDEF (prostate-derived Ets factor) | Prostate cancer | ¹²¹ |
| PSMA (prostate specific membrane antigen) | Prostate cancer | ¹²² |
| Serum Receptor-Binding Cancer Antigen (RCAS1) | Pancreatic Cancer, lung cancer, colon cancer, squamous | ^{123, 124} |
| | cell carcinoma | |
| Tyrosinase | Melanoma | ^{125, 126} |
| Prolactin (PRL) and its receptor (PRLr) | Breast cancer | ¹²⁷ |
| Beta Catenin | Ovarian cancer | ^{128, 129} |
| Matrix metalloproteinase-7 | Ovarian cancer | ¹³⁰ |
| Claudin-3 | Ovarian cancer | ^{131, 132} |
| Claudin-4 | Hepatocellular carcinoma | ^{132, 133} |
| Cyclin E | Lung and breast cancer | ^{134, 135} |
| O6-methylguanine-DNA methyltransferase (MGMT) | DNA repair protein; Downregulated in breast cancer/lung | ^{136, 137} |
| | cancer/ovarian cancer | |
| Human placental alkaline phosphatase | Germ cell tumors | ¹³⁸ |
| Calretinin | Mesothelioma/Lung cancer | ^{139, 140} |
| CD15 | Mesothelioma/Lung cancer, most carcinomas | ^{141, 142} |
| Thyroid Transcription Factor 1 | Thyroid/lung cancer | ^{143, 144} |
| Wilm's tumor 1 (WT-1) | Lung cancer | ^{145. 146} |
| MART-1 (Melan-A) | Melanoma | ^{147, 148} |
| Nucleophosmin/B23 (NPM) | Hepatoma | ¹⁴⁹ |
| p21 (Waf1/Cip1) | Tumor suppressor; Breast cancer | ^{150, 151} |
| p16 (INK4a) | Tumor suppressor; Cervical cancer | ^{152, 153} |
| Cyclin-dependent kinase inhibitor p27 (kip1) | Tumor suppressor; downregulated in numerous human tumors | ¹⁵⁴ |
| Pituitary Tumor Transforming Gene-1 | Colon cancer | ¹⁵⁵ |
| Glycosylated eosinophil-derived neurotoxin & osteopontin | Ovarian cancer | ¹⁵⁶ |
| Fibrinopeptide A | Urothelial carcinoma. ovarian cancer and gastric cancer | ¹⁵⁷ |
| Uridine phosphorylase | Breast cancer | ¹⁵⁸ |
| Modified urinary nucleosides | Multiple cancers | ¹⁵⁹ |
| Ras | Multiple cancers | ^{160, 161} |
| Myc | Multiple cancers | ^{162, 163} |
| CDKN2A | Colorectal cancer and several other cancers | ¹⁶⁴ |
| Phosphatase and tensin homolog deleted from chr. 10 | Tumor suppressor; down regulated in melanoma | ¹⁶⁵ |
| C-erbB-2 | Breast cancer | ¹⁶⁶ |
| Vascular endothelial growth factor receptor | Breast cancer | ^{167, 168, 169} |

¹ Grunkemeier M N, Vollmer R T. Predicting Prostate Biopsy Results: The Importance of PSA, Age, and Race. Am J Clin Pathol. 2006 July 126 (1): 1-3.
² Cheung R, Tucker S L, Kuban D A. First-year PSA kinetics and minima after prostate cancer radiotherapy are predictive of overall survival. Int J Radiat Oncol Biol Phys. 2006 Jun. 29; [Epub ahead of print]
³ Seki K, Miyakoshi S, Lee G H, Matsushita H, Mutoh Y, Nakase K, Ida M, Taniguchi H. Prostatic acid phosphatase is a possible tumor marker for intravascular large B-cell lymphoma. Am J Surg Pathol. 2004 October; 28(10): 1384-8.
⁴ Birtle A J, Freeman A, Masters J R, Payne H A, Harland S J: BAUS Section of Oncology Cancer Registry. Tumour markers for managing men who present with metastatic prostate cancer and serum prostate-specific antigen levels of <10 ng/mL.
⁵ Chaube A, Tewari M, Singh U, Shukla H S. CA 125: a potential tumor marker for gallbladder cancer. J Surg Oncol. 2006 Jun. 15; 93(8): 665-9.
⁶ Nakae M, Iwamoto I, Fujino T, Maehata Y, Togami S, Yoshinaga M, Douchi T. Preoperative plasma osteopontin level as a biomarker complementary to carbohydrate antigen 125 in predicting ovarian cancer. J Obstet Gynaecol Res. 2006 June; 32(3): 309-14.
⁷ Takac I, Gorisek B. Serum CA 125 levels and lymph node metastasis in patients with endometrial cancer. Wien Klin Wochenschr. 2006 May; 118 2: 62-5.
⁸ Le Page C, Ouellet V, Madore J, Hudson T J, Tonin P N, Provencher D M, Mes-Masson A M. From gene profiling to diagnostic markers: IL-18 and FGF-2 complement CA125 as serum-based markers in epithelial ovarian cancer. Int J Cancer. 2006 Apr. 1; 118(7): 1750-8.
⁹ Nicolini A, Carpi A, Tarro G. Biomolecular markers of breast cancer. Front Biosci. 2006 May 1; 11: 1818-43.
¹⁰ Bartsch R, Wenzel C, Pluschnig U, Hussian D, Sevelda U, Altorjai G, Locker G J, Mader R, Zielinski C C, Steger G G. Prognostic value of monitoring tumour markers CA 15-3 and CEA during fulvestrant treatment. BMC Cancer. 2006 Mar. 26; 6: 81.
¹¹ Snarska J, Szajda S D, Puchalski Z, Szmitkowski M, Chabielska E, Kaminski F, Zwierz P. Zwierz K. Usefulness of examination of some tumor markers in diagnostics of liver cancer. Hepatogastroenterology. 2006 March-April; 53(68): 271-4.
¹² Zhou L, Liu J, Luo F. Serum tumor markers for detection of hepatocellular carcinoma. World J Gastroenterol. 2006 Feb. 28; 12(8): 1175-81.
¹³ Cole L A, Sutton J M. Selecting an appropriate hCG test for managing gestational trophoblastic disease and cancer. J Reprod Med. 2006 March; 51(3): 217.
¹⁴ Ajufo I I, Lindow S W, Canty S H. Choriocarcinoma with markedly elevated serum hCG levels and negative urine hCG levels. J Obstet Gynaecol. 2006 January; 26(1): 83-5.
¹⁵ Cole L A, Sutton J M. Selecting an appropriate hCG test for managing gestational trophoblastic disease and cancer. J Reprod Med. 2004 July; 49(7): 545-53.
¹⁶ Nakaizumi A, Tanaka S. Tumor markers for detecting pancreatic cancer. Nippon Rinsho. 2006 January; 64 Suppl 1: 144-7.
¹⁷ Sasaki A, Kawano K, Inomata M, Shibata K, Matsumoto T, Kitano S. Value of serum carbohydrate antigen 19-9 for predicting extrahepatic metastasis in patients with liver metastasis from colorectal carcinoma. Hepatogastroenterology. 2005 November-December; 52(66): 1814-9.
¹⁸ Batlle M, Ribera J M, Oriol A, Pastor C, Mate J L, Fernandez-Aviles F, Flores A, Milla F, Feliu E. Usefulness of tumor markers CA 125 and CA 15.3 at diagnosis and during follow-up in non-Hodgkin's lymphoma: study of 200 patients. Leuk Lymphoma. 2005 October; 46(10): 1471-6.
¹⁹ Duffy M J. Serum tumor markers in breast cancer: are they of clinical value? Clin Chem. 2006 March; 52(3): 345-51. Epub 2006 Jan. 12.
²⁰ Griquer C E, Oliva C R, Gillespie G Y. Glucose metabolism heterogeneity in human and mouse malignant glioma cell lines. J Neurooncol. 2005 September; 74(2): 123-33.
²¹ Bouafia F, Drai J, Bienvenu J, Thieblemont C, Espinouse D. Salles G, Coiffier B. Profiles and prognostic values of serum LDH isoenzymes in patients with haematopoietic malignancies. Bull Cancer. 2004 July-August; 91 (7-8): E229-40.
²² Santonocito C, Concolino P, Lavieri M M, Ameglio F, Gentileschi S, Capizzi R, Rocchetti S, Amerio P, Castagnola M, Zuppi C, Capoluongo E. Comparison between three molecular methods for detection of blood melanoma tyrosinase mRNA. Correlation with melanoma stages and S100B, LDH, NSE biochemical markers. Clin Chim Acta. 2005 December; 362(1-2): 85-93.
²³ Tas F, Oguz H, Argon A, Duranyildiz D, Camlica H, Yasasever V, Topuz E. The value of serum levels of IL-6, TNF-alpha, and erythropoietin in metastatic malignant melanoma: serum IL-6 level is a valuable prognostic factor at least as serum LDH in advanced melanoma. Med Oncol. 2005; 22(3): 241-6.
²⁴ Molina R, Auge J M, Filella X, Vinolas N, Alicarte J, Domingo J M, Ballesta A M. Pro-gastrin-releasing peptide (proGRP) in patients with benign and malignant diseases: comparison with CEA, SCC, CYFRA 21-1 and NSE in patients with lung cancer. Anticancer Res. 2005 May-June; 25(3A): 1773-8.
²⁵ Ando S, Suzuki M, Yamamoto N, lida T, Kimura H. The prognostic value of both neuron-specific enolase (NSE) and Cyfra21-1 in small cell lung cancer. Anticancer Res. 2004 May-June; 24(3b): 1941-6.
²⁶ Tas F, Duranyildiz D, Oguz H, Camlica H, Yasasever V, Topuz E. The value of serum bcl-2 levels in advanced epithelial ovarian cancer. Med Oncol. 2005; 22 (2): 139-43.
²⁷ Tas F, Duranyildiz D, Oguz H, Camlica H, Oral E N, Yasasever V, Topuz E. The value of serum Bcl-2 levels in advanced lung cancer patients.
²⁸ Yaren A, Oztop I, Kargi A, Ulukus C, Onen A, Sanli A, Binicier O, Yilmaz U, Alakavuklar M. Bax, bcl-2 and c-kit expression in non-small-cell lung cancer and their effects on prognosis. Int J Clin Pract. 2006 June; 60(6): 675-82.
²⁹ Zimmerman R L, Fogt F, Goonewardene S. Diagnostic utility of BCA-225 in detecting adenocarcinoma in serous effusions. Anal Quant Cytol Histol. 2000 October; 22(5): 353-7.
³⁰ Loy T S, Chapman R K, Diaz-Arias A A, Bulatao I S, Bickel J T. Distribution of BCA-225 in adenocarcinomas. An immunohistochemical study of 446 cases. Am J Clin Pathol. 1991 September; 96(3): 326-9.
³¹ Sheu C C, Chang M Y, Chang H C, Tsai J R, Lin S R, Chang S J, Hwang J J, Huang M S, Chong I W. Combined Detection of CEA, CK-19 and c-met mRNAs in Peripheral Blood: A Highly Sensitive Panel for Potential Molecular Diagnosis of Non-Small Cell Lung Cancer. Oncology. 2006 Jun. 29; 70(3): 203-211.
³² Cheng T L, Chang M Y, Huang S Y, Sheu C C, Kao E L, Cheng Y J, Chong I W. Overexpression of circulating c-met messenger RNA is significantly correlated with nodal stage and early recurrence in non-small cell lung cancer. Chest. 2005 September; 128(3): 1453-60.
³³ Liaudet-Coopman E, Beaujouin M, Derocq D, Garcia M, Glondu-Lassis M, Laurent-Matha V, Prebois C, Rochefort H. Vignon F. Cathepsin D: newly discovered functions of a long-standing aspartic protease in cancer and apoptosis. Cancer Lett. 2006 Jun. 18; 237(2): 167-179.
³⁴ Fukuda M E, Iwadate Y, Machida T, Hiwasa T, Nimura Y, Naqai Y, Takiguchi M, Tanzawa H, Yamaura A, Seki N. Cathepsin D is a potential serum marker for poor prognosis in glioma patients. Cancer Res. 2005 Jun. 15; 65(12): 5190-4.
³⁵ Bratthauer G L, Moinfar F, Stamatakos M D, Mezzetti T P, Shekitka K M, Man Y G, Tavassoli F A. Combined E-cadherin and high molecular weight cytokeratin immunoprofile differentiates lobular, ductal, and hybrid mammary intraepithelial neoplasias. Hum Pathol. 2002 June; 33(6): 620-7.
³⁶ Raphael S J, McKeown-Eyssen G, Asa S L. High-molecular-weight cytokeratin and cytokeratin-19 in the diagnosis of thyroid tumors. Mod Pathol. 1994 April; 7(3): 295-300.
³⁷ Delektorskaya V V, Perevoshchikov A G, Golovkov D A, Kushlinskii N E. Expression of E-cadherin, beta-catenin, and CD-44v6 cell adhesion molecules in primary tumors and metastases of colorectal adenocarcinoma. Bull Exp Biol Med. 2005 June; 139(6): 706-10.
³⁸ Veveris-Lowe T L, Lawrence M G, Collard R L, Bui L, Herington A C, Nicol P L, Clements J A. Kallikrein 4 (hK4) and prostate-specific antigen (PSA) are associated with the loss of E-cadherin and an epithelial-mesenchymal transition (EMT)-like effect in prostate cancer cells. Endocr Relat Cancer. 2005 September; 12(3): 631-43.
³⁹ Fromont G, Roupret M, Amira N, Sibony M, Vallancien G, Validire P, Cussenot O. Tissue microarray analysis of the prognostic value of E-cadherin, Ki67, p53, p27, survivin and MSH2 expression in upper urinary tract transitional cell carcinoma. Eur Urol. 2005 November; 48(5): 764-70.
⁴⁰ Yamazaki K, Hasegawa M, Ohoka I, Hanami K, Asoh A, Naqao T, Sugano I, Ishida Y. Increased E2F-1 expression via tumour cell proliferation and decreased apoptosis are correlated with adverse prognosis in patients with squamous cell carcinoma of the oesophagus. J Clin Pathol. 2005 September; 58(9): 904-10.
⁴¹ Ebihara Y, Miyamoto M, Shichinohe T, Kawarada Y, Cho Y, Fukunaga A, Murakami S, Uehara H, Kaneko H, Hashimoto H, Murakami Y, Itoh T, Okushiba S, Kondo S, Katoh H. Over-expression of E2F-1 in esophageal squamous cell carcinoma correlates with tumor progression. Dis Esophagus. 2004; 17(2): 150-4.
⁴² Rishi A K, Parikh R, Wali A, Durko L, Zhang L, Yu Y, Majumdar A P. EGF receptor-related protein (ERRP) inhibits invasion of colon cancer cells and tubule formation by endothelial cells in vitro. Anticancer Res. 2006 March-April; 26(2A): 1029-37.
⁴³ Takai N, Miyazaki T, Fujisawa K, Nasu K, Miyakawa I. Expression of receptor tyrosine kinase EphB4 and its ligand ephrin-B2 is associated with malignant potential in endometrial cancer. Oncol Rep. 2001 May-June; 8(3): 567-73.
⁴⁴ Tang X X, Zhao H, Robinson M E, Cohen B, Cnaan A, London W, Cohn S L, Cheung N K, Brodeur G M, Evans A E, Ikegaki N. Implications of EPHB6, EFNB2, and EFNB3 expressions in human neuroblastoma. Proc Natl Acad Sci USA. 2000 Sep. 26; 97(20): 10936-41.
⁴⁵ Roldan G, Delqado L, Muse I M. Tumoral Expression of BRCA1, Estrogen Receptor Alpha and ID4 Protein in Patients with Sporadic Breast Cancer. Cancer Biol Ther. 2006 May; 5(5): 505-10.
⁴⁶ Lakhani S R, Reis-Filho J S, Fulford L, Penault-Llorca F, et. al. Prediction of BRCA1 status in patients with breast cancer using estrogen receptor and basal phenotype. Clin Cancer Res. 2005 Jul. 15; 11(14): 5175-80.
⁴⁷ Ding L, Erdmann C, Chinnaiyan A M, Merajver S D, Kleer C G. Identification of EZH2 as a molecular marker for a precancerous state in morphologically normal breast tissues. Cancer Res. 2006 Apr. 15; 66(8): 4095-9.
⁴⁸ Bachmann I M, Halvorsen O J, Collett K, Stefansson I M, Straume O, Haukaas S A, Salvesen H B, Otte A P, Akslen L A. EZH2 expression is associated with high proliferation rate and aggressive tumor subgroups in cutaneous melanoma and cancers of the endometrium, prostate, and breast. J Clin Oncol. 2006 Jan. 10; 24(2): 268-73.
⁴⁹ Nakata S, Sugio K, Uramoto H, Oyama T, Hanagiri T, Morita M, Yasumoto K. The methylation status and protein expression of CDH1, p16(INK4A), and fragile histidine triad in nonsmall cell lung carcinoma: epigenetic silencing, clinical features, and prognostic significance. Cancer. 2006 May 15; 106(10): 2190-9.
⁵⁰ Lea J S, Ashfaq R, Muneer S, Burbee D G, Miller D S, Minna J D, Muller C Y. Understanding the mechanisms of FHIT inactivation in cervical cancer for biomarker development. J Soc Gynecol Investig. 2004 July; 11(5): 329-37.
⁵¹ Tomos C, Soslow R, Chen S, Akram M, Hummer A J, Abu-Rustum N, Norton L, Tan L K. Expression of WT1, CA 125, and GCDFP-15 as useful markers in the differential diagnosis of primary ovarian carcinomas versus metastatic breast cancer to the ovary. Am J Surg Pathol. 2005 November; 29(11): 1482-9.
⁵² Pasquinelli R, Barba P, Capasso I, D'Aiuto M, D'Aiuto G, Anzisi A M, De Berardinis P, Guardiola J. Circulating antibodies against the breast tumor marker GCDFP-15/gp17 in mammary carcinoma patients and in patients carrying benign breast conditions. Int J Cancer. 1999 Dec. 22; 84(6): 568-72.
⁵³ Serrano-Olvera A, Duenas-Gonzalez A, Gallardo-Rincon D, Candelaria M, De la Garza-Salazar J. Prognostic, predictive and therapeutic implications of HER2 in invasive epithelial ovarian cancer. Cancer Treat Rev. 2006 May; 32(3): 180-90. Epub 2006 Feb. 17.
⁵⁴ Emlet D R, Schwartz R, Brown K A, Pollice A A, Smith C A, Shackney S E. HER2 expression as a potential marker for response to therapy targeted to the EGFR. Br J Cancer. 2006 Apr. 24; 94(8): 1144-53.
⁵⁵ Kountourakis P, Pavlakis K, Psyrri A, Rontogianni D, Xiros N, Patsouris E, Pectasides D. Economopoulos T. Prognostic significance of HER3 and HER4 protein expression in colorectal adenocarcinomas. BMC Cancer. 2006 Feb. 28; 6: 46.
⁵⁶ Edwards J, Traynor P, Munro A F, Pirret C F, Dunne B, Bartlett J M. The role of HER1-HER4 and EGFRvIII in hormone-refractory prostate cancer. Clin Cancer Res. 2006 Jan. 1; 12(1): 123-30.
⁵⁷ Ekberg T, Nestor M, Engstrom M, Nordgren H, Wester K, Carlsson J, Anniko M. Expression of EGFR, HER2, HER3, and HER4 in metastatic squamous cell carcinomas of the oral cavity and base of tongue. Int J Oncol. 2005 May; 26(5): 1177-85.
⁵⁸ Kamijima S, Tobe T, Suyama T, Ueda T, Igarashi T, Ichikawa T, Ito H. The prognostic value of p53, Ki-67 and matrix metalloproteinases MMP-2 and MMP-9 in transitional cell carcinoma of the renal pelvis and ureter. Int J Urol. 2005 November; 12(11): 941-7.
⁵⁹ Urruticoechea A, Smith I E, Dowsett M. Proliferation marker Ki-67 in early breast cancer. J Clin Oncol. 2005 Oct. 1; 23(28): 7212-20.
⁶⁰ Jin R, Huang J, Tan P H, Bay B H. Clinicopathological significance of metallothioneins in breast cancer. Pathol Oncol Res. 2004; 10(2): 74-9.
⁶¹ Cherian M G, Jayasurya A, Bay B H. Metallothioneins in human tumors and potential roles in carcinogenesis. Mutat Res. 2003 Dec. 10; 533(1-2): 201-9.
⁶² Jang K S, Paik S S, Chung H, Oh Y H, Kong G. MTA1 overexpression correlates significantly with tumor grade and angiogenesis in human breast cancers. Cancer Sci. 2006 May; 97(5): 374-9.
⁶³ Martin M D, Fischbach K, Osborne C K, Mohsin S K, Allred D C, O'Connell P. Loss of heterozygosity events impeding breast cancer metastasis contain the MTA1 gene. Cancer Res. 2001 May 1; 61(9): 3578-80.
⁶⁴ Mushenkova N, Moiseeva E, Chaadaeva A, Den Otter W, Svirshchevskaya E. Antitumor effect of double immunization of mice with mucin 1 and its coding DNA. Anticancer Res. 2005 November-December; 25(6B): 3893-8.
⁶⁵ Gao P, Zhou G Y, Zhang Q H, Xiang L, Ma C, Yu H P, Yu F. [Expression of mucin 1 and tumor invasiveness in breast carcinoma] Zhonghua Yi Xue Za Zhi. 2005 Feb. 16; 85(6): 381-4.
⁶⁶ Zeng G, Aldridge M E, Wang Y, Pantuck A J, Wang A Y, Liu Y X, Han Y, Yuan Y H, Robbins P F, Dubinett S M, deKernion J B, Belldegrun A S. Dominant B cell epitope from NY-ESO-1 recognized by sera from a wide spectrum of cancer patients: implications as a potential biomarker. Int J Cancer. 2005 Mar. 20; 114(2): 268-73.
⁶⁷ Goydos J S, Patel M, Shih W. NY-ESO-1 and CTp11 expression may correlate with stage of progression in melanoma. J Surg Res. 2001 Jun. 15; 98(2): 76-80.
⁶⁸ Santos A M, Sousa H, Pinto D, Portela C, Pereira D, Catarino R, Duarte I, Lopes C, Medeiros R. Linking TP53 codon 72 and P21 nt590 genotypes to the development of cervical and ovarian cancer. Eur J Cancer. 2006 May; 42(7): 958-63.
⁶⁹ Qiu C Z, Wang C. Huang Z X, Zhu S Z, Wu Y Y, Qiu J L. Relationship between somatostatin receptor subtype expression and clinicopathology, Ki-67, Bcl-2 and p53 in colorectal cancer. World J Gastroenterol. 2006 Apr. 7; 12(13): 2011-5.
⁷⁰ Balogh G A, Mailo D A, Corte M M, Roncoroni P. et al. Mutant p53 protein in serum could be used as a molecular marker in human breast cancer. Int J Oncol. 2006 April; 28(4): 995-1002.
⁷¹ Stephens B J, Han H, Gokhale V, Von Hoff D D. PRL phosphatases as potential molecular targets in cancer. Mol Cancer Ther. 2005 November; 4(11): 1653-61.
⁷² Kato H, Semba S, Miskad U A, Seo Y, Kasuga M, Yokozaki H. High expression of PRL-3 promotes cancer cell motility and liver metastasis in human colorectal cancer: a predictive molecular marker of metachronous liver and lung metastases. Clin Cancer Res. 2004 Nov. 1; 10(21): 7318-28.
⁷³ Theisinger B, Seitz G, Dooley S, Welter C. A second trefoil protein, ITF/hP1.B, is transcribed in human breast cancer. Breast Cancer Res Treat. 1996; 38(2): 145-51.
⁷⁴ May F E, Westley B R. Expression of human intestinal trefoil factor in malignant cells and its regulation by oestrogen in breast cancer cells. J Pathol. 1997 August; 182(4): 404-13.
⁷⁵ Ropke M, Boltze C, Meyer B, Neumann H W, Roessner A, Schneider-Stock R. Rb-loss is associated with high malignancy in chondrosarcoma. Oncol Rep. 2006 January; 15(1): 89-95.
⁷⁶ Liu T, Zhu E, Wang L, Okada T, Yamaguchi A, Okada N. Abnormal expression of Rb pathway-related proteins in salivary gland acinic cell carcinoma. Hum Pathol. 2005 September; 36(9): 962-70.
⁷⁷ Ghazizadeh M, Jin E, Shimizu H, Fujiwara M, Arai S, Ohaki Y, Takemura T, Kawanami O. Role of cdk4, p16INK4, and Rb expression in the prognosis of bronchioloalveolar carcinomas. Respiration. 2005 January-February; 72(1): 68-73.
⁷⁸ Cordon-Cardo C. p53 and RB: simple interesting correlates or tumor markers of critical predictive nature? J Clin Oncol. 2004 Mar. 15; 22(6): 975-7.
⁷⁹ Traub F, Mengel M, Luck H J, Kreipe H H, Von Wasielewski R. Prognostic impact of Skp2 and p27 in human breast cancer. Breast Cancer Res Treat. 2006 Apr. 25; [Epub ahead of print]
⁸⁰ Kamata Y, Watanabe J, Nishimura Y, Arai T, Kawaguchi M, Hattori M, Obokata A, Kuramoto H. High expression of skp2 correlates with poor prognosis in endometrial endometrioid adenocarcinoma. J Cancer Res Clin Oncol. 2005 September; 131(9): 591-6.
⁸¹ Li J Q, Wu F, Masaki T, Kubo A. Fujita J, Dixon D A, Beauchamp R D, Ishida T, Kuriyama S, Imaida K. Correlation of Skp2 with carcinogenesis, invasion, metastasis, and prognosis in colorectal tumors. Int J Oncol. 2004 July; 25(1): 87-95.
⁸² Kang H Y, Lin H K, Hu Y C, Yeh S, Huang K E, Chang C. From transforming growth factor-beta signaling to androgen action: identification of Smad3 as an androgen receptor coregulator in prostate cancer cells. Proc Natl Acad Sci USA. 2001 Mar. 13; 98(6): 3018-23.
⁸³ Kusaba T, Nakayama T, Yamazumi K, Yakata Y, Yoshizaki A, Inoue K, Nagayasu T, Sekine I. Activation of STAT3 is a marker of poor prognosis in human colorectal cancer. Oncol Rep. 2006 June; 15(6): 1445-51.
⁸⁴ Lai R, Navid F, Rodriguez-Galindo C, Liu T, Fuller C E, Ganti R, Dien J, Dalton J, Billups C, Khoury J D. STAT3 is activated in a subset of the Ewing sarcoma family of tumours. J Pathol. 2006 April; 208(5): 624-32.
⁸⁵ Hsiao J R, Jin Y T, Tsai S T, Shiau A L, Wu C L, Su W C. Constitutive activation of STAT3 and STAT5 is present in the majority of nasopharyngeal carcinoma and correlates with better prognosis. Br J Cancer. 2003 Jul. 21; 89 (2): 344-9.
⁸⁶ Correale M, Abbate I, Catino A, Tedone T, Musci M D, Dragone D, Gargano G, Izzi G, Cramarossa A. Serum levels of TAG 72 and CA 15.3 in patients with gynaecological neoplasms. Eur J Gynaecol Oncol. 1992; 13(1 Suppl): 82-4.
⁸⁷ Correale M, Abbate I, Gargano G, Garrubba M, Muncipinto A, Addabbo L, Colangelo D. Serum TAG 72 levels in different human carcinomas. Int J Rad Appl Instrum B. 1991; 18(1): 101-3.
⁸⁸ Motoo Y, Sawabu N. Clinical significance of TAG-72 assay as a tumor marker. Nippon Rinsho. 1990 February; 48 Suppl: 1064-7.
⁸⁹ Gazzaniga P, Nofroni I, Gandini O, Silvestri I, Frati L, Agliano A M, Gradilone A. Tenascin C and epidermal growth factor receptor as markers of circulating tumoral cells in bladder and colon cancer. Oncol Rep. 2005 November; 14(5): 1199-202.
⁹⁰ Sedele M, Karaveli S, Pestereli H E, Simsek T, Elpek G, Uner M, Sargin C F. Tenascin expression in normal, hyperplastic, and neoplastic endometrium. Int J Gynecol Pathol. 2002 April; 21(2): 161-6.
⁹¹ Span P N, Lindberg R L, Manders P, Tjan-Heijnen V C, Heuvel J J, Beex L V. Sweep C G. Tissue inhibitors of metalloproteinase expression in human breast cancer: TIMP-3 is associated with adjuvant endocrine therapy success. J Pathol. 2004 April; 202(4): 395-402.
⁹² Ishihara Y, Nishikawa T, Iijima H, Matsunaqa K. Expression of matrix metalloproteinase, tissue inhibitors of metalloproteinase and adhesion molecules in silicotic mice with lung tumor metastasis. Toxicol Lett. 2003 Apr. 30; 142(1-2): 71-5.
⁹³ Mano M S, Awada A, Di Leo Am Durbecq V, Paesmans Mm Cardoso F, Larsimont D, Piccart M. Rates of topoisomerase II-alpha and HER-2 gene amplification and expression in epithelial ovarian carcinoma. Gynecol Oncol. 2004 March; 92(3): 887-95.
⁹⁴ Yang D T, Holden J A, Florell S R. CD117, CK20, TTF-1, and DNA topoisomerase II-alpha antigen expression in small cell tumors. J Cutan Pathol. 2004 March; 31(3): 254-61.
⁹⁵ Cardoso F, Durbecq V, Larsimont D, Paesmans M, Leroy J Y, Rouas G, Sotiriou C, Renard N, Richard V, Piccart M J, Di Leo A. Correlation between complete response to anthracycline-based chemotherapy and topoisomerase II-alpha gene amplification and protein overexpression in locally advanced/metastatic breast cancer. Int J Oncol. 2004 January; 24(1): 201-9.
⁹⁶ Ecke T H, Schlechte H H, Schulze G, Lenk S V, Loening S A. Four tumour markers for urinary bladder cancer-tissue polypeptide antigen (TPA), HER-2/neu (ERB B2), urokinase-type plasminogen activator receptor (uPAR) and TP53 mutation. Anticancer Res. 2005 January-February; 25(1B): 635-41.
⁹⁷ Werle B, Kotzsch M, Lah T T, Kos J, Gabrijelcic-Geiger D. Spiess E, Schirren J, Ebert W, Fiehn W, Luther T, Magdolen V, Schmitt M, Harbeck N. Cathepsin B, plasminogenactivator-inhibitor (PAI-1) and plasminogenactivator-receptor (uPAR) are prognostic factors for patients with non-small cell lung cancer. Anticancer Res. 2004 November-December; 24(6): 4147-61.
⁹⁸ Tsuzuki H, Sunaga H, Ito T, Narita N, Sugimoto C, Fujieda S. Reliability of platelet-derived endothelial cell growth factor as a prognostic factor for oral and oropharyngeal carcinomas. Arch Otolaryngol Head Neck Surg. 2005 December; 131(12): 1071-8.
⁹⁹ Mizutani Y, Wada H, Yoshida O, Fukushima M, Kawauchi A, Nakao M, Miki T. The significance of thymidine phosphorylase/platelet-derived endothelial cell growth factor activity in renal cell carcinoma. Cancer. 2003 Aug. 15; 98(4): 730-6.
¹⁰⁰ Leonard J H, Marks L H, Chen W, Cook A L, Boyle G M, Smit D J, Brown D L, Stow J L, Parsons P G, Sturm R A. Screening of human primary melanocytes of defined melanocortin-1 receptor genotype: pigmentation marker, ultrastructural and UV-survival studies. Pigment Cell Res. 2003 June; 16(3): 198-207.
¹⁰¹ Salazar-Onfray F, Lopez M, Lundqvist A, Aguirre A, Escobar A, Serrano A, Korenblit C, Petersson M, Chhajlani V, Larsson O, Kiessling R. Tissue distribution and differential expression of melanocortin 1 receptor, a malignant melanoma marker. Br J Cancer. 2002 Aug. 12; 87(4): 414-22.
¹⁰² Zielie P J, Mobley J A, Ebb R G, Jiang Z, Blute R D, Ho S M. A novel diagnostic test for prostate cancer emerges from the determination of alpha-methylacyl-coenzyme a racemase in prostatic secretions. J Urol. 2004 September; 172(3): 1130-3.
¹⁰³ Jiang Z, Li C, Fischer A, Dresser K, Woda B A. Using an AMACR (P504S)/34betaE12/p63 cocktail for the detection of small focal prostate carcinoma in needle biopsy specimens. Am J Clin Pathol. 2005 February; 123(2): 231-6.
¹⁰⁴ Molinie V, Herve J M, Lugagne P M, Lebret T, Botto H. Diagnostic utility of a p63/alpha-methyl coenzyme A racemase (p504s) cocktail in ambiguous lesions of the prostate upon needle biopsy. BJU Int. 2006 May; 97(5): 1109-15.
¹⁰⁵ Kunju L P, Mehra R, Snyder M, Shah R B. Prostate-specific antigen, high-molecular-weight cytokeratin (clone 34betaE12), and/or p63: an optimal immunohistochemical panel to distinguish poorly differentiated prostate adenocarcinoma from urothelial carcinoma. Am J Clin Pathol. 2006 May; 125(5): 675-81.
¹⁰⁶ Yaren A, Oztop I, Kargi A, Ulukus C, Onen A, Sanli A, Binicier O, Yilmaz U, Alakavuklar M. Bax, bcl-2 and c-kit expression in non-small-cell lung cancer and their effects on prognosis. Int J Clin Pract. 2006 June; 60(6): 675-82.
¹⁰⁷ Kamakura Y, Hasegawa M, Minamoto T, Yamashita J, Fujisawa H. C-kit gene mutation: common and widely distributed in intracranial germinomas. J Neurosurg. 2006 March; 104(3 Suppl): 173-80.
¹⁰⁸ Perrone G, Santini D, Verzi A, Vincenzi B, Borzomati D, Vecchio F, Coppola R, Antinori A, Magistrelli P, Tonini G, Rabitti C. COX-2 expression in ampullary carcinoma: correlation with angiogenesis process and clinicopathological variables. J Clin Pathol. 2006 May; 59(5): 492-6. Epub 2006 Feb. 17.
¹⁰⁹ Ferrandina G, Ranelletti F O, Gallotta V, Martinelli E, Zannoni G F, Gessi M, Scambia G. Expression of cyclooxygenase-2 (COX-2), receptors for estrogen (ER), and progesterone (PR), p53, ki67, and neu protein in endometrial cancer. Gynecol Oncol. 2005 September; 98(3): 383-9.
¹¹⁰ Yeh T S, Tseng J H, Liu N J, Chen T C, Jan Y Y, Chen M F. Significance of cellular distribution of ezrin in pancreatic cystic neoplasms and ductal adenocarcinoma. Arch Surg. 2005 December; 140(12): 1184-90.
¹¹¹ Ilmonen S, Vaheri A, Asko-Seljavaara S, Carpen O. Ezrin in primary cutaneous melanoma. Mod Pathol. 2005 April; 18(4): 503-10.
¹¹² Lee C S, Southey M C, Waters K, Kannourakis G, Georgiou T, Armes J E, Chow C W, Venter D J. EWS/FLI-1 fusion transcript detection and MIC2 immunohistochemical staining in the diagnosis of Ewing's sarcoma. Pediatr Pathol Lab Med. 1996 May-June; 16(3): 379-92.
¹¹³ Okada K, Shimura T, Suehiro T, Mochiki E, Kuwano H. Reduced galectin-3 expression is an indicator of unfavorable prognosis in gastric cancer. Anticancer Res. 2006 March-April; 26(2B): 1369-76.
¹¹⁴ Gillis J S. Microarray evidence of glutaminyl cyclase gene expression in melanoma: Implications for tumor antigen specific immunotherapy. J Transl Med. 2006 Jul. 4; 4(1): 27
¹¹⁵ Qiu G, Fang J, He Y. 5′ CpG island methylation analysis identifies the MAGE-A1 and MAGE-A3 genes as potential markers of HCC. Clin Biochem. 2006 March; 39 (3): 259-66.
¹¹⁶ Hellstrom I, Raycraft J, Kanan S, Sardesai N Y, Verch T, Yang Y, Hellstrom K E. Mesothelin variant 1 is released from tumor cells as a diagnostic marker. Cancer Epidemiol Biomarkers Prev. 2006 May; 15(5): 1014-20.
¹¹⁷ Hassan R, Remaley A T, Sampson M L, Zhang J, Cox D D, Pingpank J, Alexander R, Willingham M, Pastan I, Onda M. Detection and quantitation of serum mesothelin, a tumor marker for patients with mesothelioma and ovarian cancer. Clin Cancer Res. 2006 Jan. 15; 12(2): 447-53.
¹¹⁸ Diamandis E P, Yousef G M. Human tissue kallikreins: a family of new cancer biomarkers. Clin Chem. 2002 August; 48(8): 1198-205.
¹¹⁹ Yousef G M, Diamandis E P. An overview of the kallikrein gene families in humans and other species: emerging candidate tumour markers. Clin Biochem. 2003 September; 36(6): 443-52.
¹²⁰ Tiacci E, Pileri S, Orleth A, Pacini R, Tabarrini A, Frenguelli F, Liso A, Diverio D, Lo-Coco F, Falini B. PAX5 expression in acute leukemias: higher B-lineage specificity than CD79a and selective association with t(8; 21)-acute myelogenous leukemia. Cancer Res. 2004 Oct. 15; 64(20): 7399-404.
¹²¹ Oettgen P, Finger E, Sun Z, Akbarali Y, Thamrongsak U, Boltax J, Grall F, Dube A, Weiss A, Brown L, Quinn G, Kas K, Endress G, Kunsch C, Libermann T A. PDEF, a novel prostate epithelium-specific ets transcription factor, interacts with the androgen receptor and activates prostate-specific antigen gene expression. J Biol Chem. 2000 Jan. 14; 275(2): 1216-25.
¹²² Dumas F, Gala J L, Berteau P, Brasseur F, Eschwege P, Paradis V, Lacour B, Philippe M, Loric S. Molecular expression of PSMA mRNA and protein in primary renal tumors. Int J Cancer. 1999 Mar. 15; 80(6): 799-803.
¹²³ Ozkan H, Akar T, Koklu S, Coban S. Significance of Serum Receptor-Binding Cancer Antigen (RCAS1) in Pancreatic Cancer and Benign Pancreatobiliary Diseases. Pancreatology. 2006 Apr. 19; 6(4): 268-272
¹²⁴ Leelawat K, Watanabe T, Nakajima M, Tujinda S, Suthipintawong C, Leardkamolkarn V. Upregulation of tumour associated antigen RCAS1 is implicated in high stages of colorectal cancer. J Clin Pathol. 2003 October; 56(10): 764-8.
¹²⁵ Glumac N, Snoj M, Hocevar M, Novakovic S. Prognostic significance of tyrosinase mRNA detected by nested RT-PCR in patients with malignant melanoma. Neoplasma. 2006; 53(1): 9-14.
¹²⁶ Garbe C, Leiter U, Ellwanger U, Blaheta H J, Meier F, Rassner G, Schittek B. Diagnostic value and prognostic significance of protein S-100beta, melanoma-inhibitory activity, and tyrosinase/MART-1 reverse transcription-polymerase chain reaction in the follow-up of high-risk melanoma patients. Cancer. 2003 Apr. 1; 97(7): 1737-45.
¹²⁷ Bhatavdekar J M, Patel D D, Shah N G, Vora H H, Suthar T P, Ghosh N, Chikhlikar P R, Trivedi T I. Prolactin as a local growth promoter in patients with breast cancer: GCRI experience. Eur J Surg Oncol. 2000 September; 26(6): 540-7.
¹²⁸ Settakorn J, Kaewpila N, Burns G F, Leong A S. FAT, E-cadherin, beta catenin, HER 2/neu, Ki67 immuno-expression, and histological grade in intrahepatic cholangiocarcinoma. J Clin Pathol. 2005 December; 58(12): 1249-54.
¹²⁹ Montgomery E, Folpe A L. The diagnostic value of beta-catenin immunohistochemistry. Adv Anat Pathol. 2005 November; 12(6): 350-6.
¹³⁰ Zhang J, Jin X, Fang S, Wang R, Li Y, Wang N, Guo W, Wang Y, Wen D, Wei L, Dong Z, Kuang G. The functional polymorphism in the matrix metalloproteinase-7 promoter increases susceptibility to esophageal squamous cell carcinoma, gastric cardiac adenocarcinoma and non-small cell lung carcinoma. Carcinogenesis. 2005 October; 26(10): 1748-53.
¹³¹ Montgomery E, Mamelak A J, Gibson M, Maitra A, Sheikh S, Amr S S, Yang S, Brock M, Forastiere A, Zhang S, Murphy K M, Berg K D. Overexpression of claudin proteins in esophageal adenocarcinoma and its precursor lesions. Appl Immunohistochem Mol Morphol. 2006 March; 14(1): 24-30.
¹³² Soini Y. Claudins 2, 3, 4, and 5 in Paget's disease and breast carcinoma. Hum Pathol. 2004 December; 35(12): 1531-6.
¹³³ Lodi C, Szabo E, Holczbauer A, Batmunkh E, Szijarto A, Kupcsulik P, Kovalszky I. Paku S, Illyes G, Kiss A, Schaff Z. Claudin-4 differentiates biliary tract cancers from hepatocellular carcinomas. Mod Pathol. 2006 March; 19(3): 460-9.
¹³⁴ Sieuwerts A M, Look M P, Meijer-van Gelder M E, Timmermans M, Trapman A M, Garcia R R, Arnold M, Goedheer A J, de Weerd V, Portengen H, Klijn J G, Foekens J A. Which cyclin E prevails as prognostic marker for breast cancer? Results from a retrospective study involving 635 lymph node-negative breast cancer patients. Clin Cancer Res. 2006 Jun. 1; 12(11 Pt 1): 3319-28.
¹³⁵ Lopez-Beltran A, MacLennan G T, Montironi R. Cyclin E as molecular marker in the management of breast cancer: a review. Anal Quant Cytol Histol. 2006 April; 28(2): 111-4.
¹³⁶ Brell M, Tortosa A, Verger E, Gil J M, Vinolas N, Villa S, Acebes J J, Caral L, Pujol T, Ferrer I, Ribalta T, Graus F. Prognostic significance of O6-methylguanine-DNA methyltransferase determined by promoter hypermethylation and immunohistochemical expression in anaplastic gliomas. Clin Cancer Res. 2005 Jul. 15; 11(14) 5167-74.
¹³⁷ Akcay T, Dincer Y. Alademir Z, Aydinli K, Arvas M, Demirkiran F, Kosebay D. Significance of the O6-methylguanine-DNA methyltransferase and glutathione S-transferase activity in the sera of patients with malignant and benign ovarian tumors. Eur J Obstet Gynecol Reprod Biol. 2005 Mar. 1; 119(1): 108-13.
¹³⁸ De Broe M E, Pollet D E. Multicenter evaluation of human placental alkaline phosphatase as a possible tumor-associated antigen in serum. Clin Chem. 1988 October; 34(10): 1995-9.
¹³⁹ Cates J M, Coffing B N, Harris B T, Black C C. Calretinin expression in tumors of adipose tissue. Hum Pathol. 2006 March; 37(3): 312-21.
¹⁴⁰ Cathro H P, Stoler M H. The utility of calretinin, inhibin, and WT1 immunohistochemical staining in the differential diagnosis of ovarian tumors. Hum Pathol. 2005 February; 36(2): 195-201.
¹⁴¹ Barry T S, Jaffe E S, Sorbara L, Raffeld M, Pittaluga S. Peripheral T-cell lymphomas expressing CD30 and CD15. Am J Surg Pathol. 2003 December; 27(12): 1513-22.
¹⁴² Comin C E, Novelli L, Boddi V, Paglierani M, Dini S. Calretinin, thrombomodulin, CEA, and CD15: a useful combination of immunohistochemical markers for differentiating pleural epithelial mesothelioma from peripheral pulmonary adenocarcinoma. Hum Pathol. 2001 May; 32(5): 529-36.
¹⁴³ Barlesi F, Pinot D, Legoffic A, Doddoli C, Chetaille B, Torre J P, Astoul P. Positive thyroid transcription factor 1 staining strongly correlates with survival of patients with adenocarcinoma of the lung. Br J Cancer. 2005 Aug. 22; 93(4): 450-2.
¹⁴⁴ Zamecnik J, Chanova M, Kodet R. Expression of thyroid transcription factor 1 in primary brain tumours. J Clin Pathol. 2004 October; 57(10): 1111-3.
¹⁴⁵ Hossain A, Nixon M, Kuo M T, Saunders G F. N-terminally truncated WT1 protein with oncogenic properties overexpressed in leukemia. J Biol Chem. 2006 May 12 [Epub ahead of print]
¹⁴⁶ Hatta Y, Takeuchi J, Saitoh T, Itoh T, Ishizuka H, Iriyama N, Miyajima T, Kaneita Y, Saiki M, Yasukawa K, Yasukawa R, Kura Y, Nishinarita S. Sawada U, Horie T. WT1 expression level and clinical factors in multiple myeloma. J Exp Clin Cancer Res. 2005 December; 24(4): 595-9.
¹⁴⁷ Kounalakis N, Goydos J S. Tumor cell and circulating markers in melanoma: diagnosis, prognosis, and management. Curr Oncol Rep. 2005 September; 7(5): 377-82.
¹⁴⁸ Zubovits J, Buzney E, Yu L, Duncan L M. HMB-45, S-100, NK1/C3, and MART-1 in metastatic melanoma. Hum Pathol. 2004 February; 35(2): 217-23
¹⁴⁹ Grisendi S, Mecucci C, Falini B, Pandolfi P P. Nucleophosmin and cancer. Nat Rev Cancer. 2006 July; 6(7): 493-505.
¹⁵⁰ Santos A M, Sousa H, Pinto D, Portela C, Pereira D, Catarino R, Duarte I, Lopes C, Medeiros R. Linking TP53 codon 72 and P21 nt590 genotypes to the development of cervical and ovarian cancer. Eur J Cancer. 2006 May; 42(7): 958-63.
¹⁵¹ Li G, Liu Z, Sturgis E M, Shi Q, Chamberlain R M, Spitz M R, Wei Q. Genetic polymorphisms of p21 are associated with risk of squamous cell carcinoma of the head and neck. Carcinogenesis. 2005 September; 26(9): 1596-602.
¹⁵² Papadimitrakopoulou V, Izzo J, Lippman S M, Lee J S, Fan Y H, Clayman G, Ro J Y, Hittelman W N, Lotan R, Hong W K, Mao L. Frequent inactivation of p16INK4a in oral premalignant lesions. Oncogene. 1997 Apr. 17; 14(15): 1799-803
¹⁵³ Sabah M, Cummins R, Leader M, Kay E. Loss of p16 (INK4A) expression is associated with allelic imbalance/loss of heterozygosity of chromosome 9p21 in microdissected malignant peripheral nerve sheath tumors. Appl Immunohistochem Mol Morphol. 2006 March; 14(1): 97-102.
¹⁵⁴ Hershko D D, Shapira M. Prognostic role of p27(Kip1) deregulation in colorectal cancer. Cancer. 2006 Jul. 6; [Epub ahead of print]
¹⁵⁵ Zhu X, Mao Z, Na Y, Quo Y, Wang X, Xin D. Significance of pituitary tumor transforming gene 1 (PTTG1) in prostate cancer. Anticancer Res. 2006 March-April; 26(2A): 1253-9.
¹⁵⁶ Ye B, Skates S, Mok S C, Horick N K, Rosenberg H F, Vitonis A, Edwards D, Sluss P, Han W K, Berkowitz R S, Cramer D W. Proteomic-based discovery and characterization of glycosylated eosinophil-derived neurotoxin and COOH-terminal osteopontin fragments for ovarian cancer in urine. Clin Cancer Res. 2006 Jan. 15; 12(2): 432-41.
¹⁵⁷ Theodorescu D, Wittke S, Ross M M, Walden M, Conaway M, Just I. Mischak H, Frierson H F. Discovery and validation of new protein biomarkers for urothelial cancer: a prospective analysis. Lancet Oncol. 2006 March; 7(3): 230-40.
¹⁵⁸ Miyashita H, Takebayashi Y, Eliason J F, Fujimori F, Nitta Y, Sato A, Morikawa H, Ohashi A, Motegi K, Fukumoto M, Mori S, Uchida T. Uridine phosphorylase is a potential prognostic factor in patients with oral squamous cell carcinoma. Cancer. 2002 Jun. 1; 94(11): 2959-66.
¹⁵⁹ Feng B, Zheng M H, Zheng Y F, Lu A G, Li J W, Wang M L, Ma J J, Xu G W, Liu B Y, Zhu Z G. Normal and modified urinary nucleosides represent novel biomarkers for colorectal cancer diagnosis and surgery monitoring. J Gastroenterol Hepatol. 2005 December; 20(12): 1913-9.
¹⁶⁰ Kranenburg O. The KRAS oncogene: past, present, and future. Biochim Biophys Acta. 2005 Nov. 25; 1756(2): 81-2.
¹⁶¹ Deramaudt T, Rustgi A K. Mutant KRAS in the initiation of pancreatic cancer. Biochim Biophys Acta. 2005 Nov. 25; 1756(2): 97-101.
¹⁶² Ponzielli R, Katz S, Barsyte-Lovejoy D, Penn L Z. Cancer therapeutics: targeting the dark side of Myc. Eur J Cancer. 2005 November; 41(16): 2485-501.
¹⁶³ Popescu N C, Zimonjic D B. Chromosome-mediated alterations of the MYC gene in human cancer. J Cell Mol Med. 2002 April-June; 6(2): 151-9.
¹⁶⁴ Debniak T, Gorski B, Huzarski T, Byrski T, Cybulski C, Mackiewicz A, et al. A common variant of CDKN2A (p16) predisposes to breast cancer. J Med Genet. 2005 October; 42(10): 763-5.
¹⁶⁵ Bilbao C, Rodriguez G, Ramirez R, Falcon O, Leon L, Chirino R, Rivero J F, Falcon O Jr, Diaz-Chico B N, Diaz-Chico J C, Perucho M. The relationship between microsatellite instability and PTEN gene mutations in endometrial cancer. Int J Cancer. 2006 Aug. 1; 119(3): 563-70.
¹⁶⁶ Califano D, Losito S, Pisano C, Santelli G, Greggi S, Iodice F, DiVagno G, Silvestro G, Tambaro R, Formato R, Iaffaioli V R, Di Maio M, Pignata S. Significance of erb-B2 immunoreactivity in cervical cancer. Front Biosci. 2006 Sep. 1; 11: 2071-6.
¹⁶⁷ Swelam W, Ida-Yonemochi H, Maruyama S, Ohshiro K, Cheng J, Saku T. Vascular endothelial growth factor in salivary pleomorphic adenomas: one of the reasons for their poorly vascularized stroma. Virchows Arch. 2005 June; 446 (6): 653-62.
¹⁶⁸ Ueno T, Chow L W, Toi M. Increases in circulating VEGF levels during COX-2 inhibitor treatment in breast cancer patients. Biomed Pharmacother. 2006 Jun. 23; [Epub ahead of print]
¹⁶⁹ Des Guetz G, Uzzan B, Nicolas P, Cucherat M, Morere J F, Benamouzig R, Breau J L, Perret G Y. Microvessel density and VEGF expression are prognostic factors in colorectal cancer. Meta-analysis of the literature. Br J Cancer. 2006 Jun. 19; 94(12): 1823-32.

1. A method of determining a presence of a cancer marker in a sample comprising:
(a) mixing said sample with capture-associated oligos conjugated to capture moieties specific for said cancer marker, thereby producing a first mixture comprising reacted capture-associated oligo complexes that are associated with said cancer marker and unreacted capture-associated oligo complexes that are not associated with said cancer marker;
(b) contacting said first mixture with immobilized binding partners, wherein said immobilized binding partners facilitate separation of said unreacted capture-associated oligo complexes from said reacted capture-associated oligo complexes to produce a second mixture comprising said unreacted capture-associated oligo complexes and a third mixture comprising said reacted capture-associated oligo complexes;
(c) providing a detection device comprising oligos complementary to said capture-associated oligos, wherein said detection device produces a signal if there is a hybridization event between said capture-associated oligos and said oligos complementary to said capture-associated oligos;
(d) introducing said third mixture to said detection device; and
(e) detecting said signal, wherein said signal is indicative of said presence of said cancer marker in said sample.

2. A method of detecting the presence of at least one cancer marker in a sample, said method comprising:
a) forming a first complex by mixing said sample with capture-associated oligo(s), wherein each capture-associated oligo comprises a capture moiety specific for the cancer marker to be detected, an amplification moiety to enable amplification, and a sequence that is the same or substantially identical to a chip-associated oligo;
b) isolating said first complex from the surrounding solution;
c) amplification of at least part of the capture-associated oligo(s) to form polymerization products with a sequence the same as or substantially identical to the chip-associated oligo(s);
d) contacting the polymerization products with chip-associated oligo(s) to allow hybridization;
e) detection of the hybridization,
wherein detection of the hybridization indicates that at least one cancer marker was present in the sample.

3. The method of claim 2, wherein said amplification moiety is a promoter.

4. The method of claim 2, wherein said amplification moiety is a PCR primer site.

5. The method of claim 2, wherein said isolating first said complex from the surrounding solution occurs by use of immobilized binding partners.

6. The method of claim 2, wherein said polymerization products are RNA sequences.

7. The method of claim 2, wherein said detection occurs by electrochemical detection.

8. The method of claim 7, wherein said electrochemical detection comprises the use of one or more hybridization indicators.

9. The method of claim 8, wherein the one or more hybridization indicators is selected from the group consisting of: intercalating agents, minor groove binding agents, conjugated antibodies and other nucleic acid binding agents.

10. The method of claim 8, wherein the two or more hybridization indicators used are identical.

11. The method of claim 8, wherein the two or more hybridization indicators used are different from one another.

12. The method of claim 2, wherein said amplification is isothermal amplification.

13. The method of claim 2, wherein said capture-associated oligo(s) encodes a sequence at its 3′ end that is complementary or substantially complementary to a polymerase recognition sequence.

14. The method of claim 2, said method comprising more than one type of capture-associated oligo(s).

15. A method of detecting the presence of at least one cancer marker in a sample, said method comprising:
a) forming a first complex by mixing said sample with capture-associated oligo(s), wherein each capture-associated oligo comprises:
i) a capture moiety specific for the cancer marker to be detected;
ii) an amplification moiety to enable amplification;
iii) a sequence at its 3′ end complementary or substantially complementary to a polymerase recognition sequence; and,
iv) a sequence that is the same or substantially identical to a chip-associated oligo;
b) isolating said first complex from the surrounding solution;
c) contacting the first complex with a priming oligonucleotide that is complementary or substantially complementary to the 5′ to 3′ polymerase recognition sequence to form a double-stranded polymerase recognition site;
d) addition of an excess of mononucleotides and polymerase(s);
e) at least one round of amplification of at least part of the capture-associated oligo(s) to form polymerization products with a sequence the same as or substantially identical to the chip-associated oligo(s);
f) contacting the polymerization products with the chip-associated oligos to allow hybridization;
g) detection of the hybridization, wherein detection of the hybridization indicates that at least one cancer marker was present in the sample.

16. The method of claim 15, wherein said polymerase recognition site is a phage-encoded RNA polymerase recognition site.

17. The method of claim 15, said method comprising more than one type of capture-associated oligo(s).

18. The method of claim 15, wherein hybridization is detected by use of antibody reagents capable of binding to DNA:RNA or RNA: RNA duplexes.

19. The method of claim 18, wherein said antibody reagent is labeled with a moiety.

20. The method of claim 19, wherein said moiety is selected from the group consisting of enzymatically active group, fluorescer, chromophore, luminescer, specifically bindable ligand, electrochemically detectable molecule, and radioisotope.

21. A method of detecting the presence of at least one cancer marker in a sample, said method comprising:
a) forming a first complex by mixing said sample with capture-associated oligo(s), wherein each capture-associated oligo comprises:
i) a capture moiety specific for the cancer marker to be detected;
ii) an amplification moiety to enable amplification; and,
iii) a sequence that is the same or substantially identical to a chip-associated oligo;
b) contacting the first complex with an immobilized binding partner to the cancer marker, thereby forming a second mixture comprising a solution phase and an immobilized phase, wherein the immobilized phase comprises a capture oligo-cancer marker-immobilized binding partner complex;
c) isolating the immobilized phase from the solution phase and discarding the solution phase;
d) releasing the immobilized phase into a second solution;
e) transferring the second solution to a detection device; and
f) detecting the immobilized phase'
wherein detection of the immobilized phase indicates that at least one cancer marker was present in the sample.

22. The method of claim 21, further comprising the step of releasing the oligo from the capture oligo-cancer marker-immobilized binding partner complex.

23. The method of claim 21, further comprising the steps of:
a) amplification of at least part of the capture-associated oligo(s) to form polymerization products with a sequence the same as or substantially identical to the chip-associated oligo(s);
b) contacting the polymerization products with chip-associated oligo(s) to allow hybridization;
c) detection of the hybridization,
wherein detection of the hybridization indicates that at least one cancer marker was present in the sample.
